SEQUENCE LISTING (2 sequences)

SEQ ID NO: 1 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 2 - protein, 328 aa, Homo sapiens
MTGVFDSLVADMHSTQIAASSTYHQHQQPPSGGGAGPGGNSSSSSSLHKPQESPTLPVSTATDSSYYTNQQHPAGGGGGGGSPYAHMGSYQYQASGLNNVPYSAKSSYDLGYTAAYTSYAPYGTSSSPANNEPEKEDLEPEIRIVNGKPKKVRKPRTIYSSFQLAALQRRFQKTQYLALPERAELAASLGLTQTQVKIWFQNRRSKFKKMWKSGEIPSEQHPGASASPPCASPPVSAPASWDFGVPQRMAGGGGPGSGGSGAGSSGSSPSSAASAFLGNYPWYHQTSGSASHLQATAPLLHPTQTPQPHHHHHHHGGGGAPVSAGTIF

(19) United States
(12) Patent Application Publication
(10) Pub. No.: US 2020/0405801 A1
(43) Pub. Date: Dec. 31, 2020

(54) METHODS AND MATERIALS FOR TREATING HUNTINGTON'S DISEASE

(71) Applicant: The Penn State Research Foundation, University Park, PA (US)

(72) Inventors: Gong Chen, State College, PA (US); Zheng Wu, State College, PA (US); Ziyuan Guo, Lexington, KY (US); Yuchen Chen, Bellefonte, PA (US); Zifei Pei, State College, PA (US)

(21) Appl. No.: 16/903,611

(22) Filed: Jun. 17, 2020

Related U.S. Application Data
(60) Provisional application No. 62/868,499, filed on Jun. 28, 2019.

(51) Int. Cl. A61K 38/02; A61P 43/00; C12N 5/079; A61K 48/00; C12N 15/86

(52) U.S. Cl. CPC A61K 38/02; A61P 43/00; C12N 15/86; A61K 48/00; C12N 5/0622

(57) ABSTRACT

This document provides methods and materials for treating a mammal having Huntington's disease. For example, methods and materials for forming GABAergic neurons that are functionally integrated into the brain of a living mammal (e.g., a human) and/or for modifying one or both huntingtin (Htt) genes (or HTT RNAs or HTT polypeptides) present in a mammal with Huntington's disease are provided.

### CROSS-REFERENCE TO RELATED APPLICATIONS

This application claims the benefit of U.S. Patent Application Ser. No. 62/868,499, filed on Jun. 28, 2019. The disclosure of the prior application is considered part of (and is incorporated by reference in) the disclosure of this application.

### STATEMENT AS TO FEDERALLY SPONSORED RESEARCH

This invention was made with government support under Grant No. AG045656 awarded by the National Institutes of Health. The Government has certain rights in the invention.

### BACKGROUND

### 1. Technical Field

This document relates to methods and materials for treating a mammal having Huntington's disease. For example, this document provides methods and materials for generating striatal medium spiny neurons (MSNs) that are functionally integrated into the brain of a living mammal (e.g., a human) and for modifying one or both huntingtin (Htt) genes present in a mammal with Huntington's disease.

### 2. Background Information

Huntington's disease is mainly caused by mutations in the Htt gene, resulting in the expansion of trinucleotide CAG repeats in the Htt gene that encode polyglutamine expansions in the HTT polypeptide. When the number of CAG repeats in a Htt gene exceeds 36, it will cause disease, and the MSNs in the striatum are in particular vulnerable to such polyglutamine toxicity (Ross et al., Lancet Neurol., 10:83-98 (2011); and Walker, Lancet, 369:218-228 (2007)). Currently, there is no effective treatment for Huntington's disease due to the combinatorial effects of mutant HTT toxicity and the neuronal loss.

### SUMMARY

This document provides methods and materials for treating a mammal having Huntington's disease through regeneration of functional new neurons and reduction of mutant HTT toxicity. For example, nucleic acid encoding a NeuroD1 polypeptide and nucleic acid encoding a Dlx2 polypeptide can be used to convert glial cells (e.g., reactive astrocytes) within the brain (e.g., striatum) into striatal MSNs (e.g., astrocyte-converted neurons) that are functionally integrated into the brain of a living mammal (e.g., a human) with Huntington's disease, and one or more gene therapy components (e.g., a nuclease, a targeting sequence such antisense oligonucleotides or guide RNAs, and/or a donor nucleic acid) designed to modify one or more Htt alleles (or its transcribed HTT RNAs or translated HTT polypeptides) within one or more glial cells (e.g., reactive astrocytes) and/or one or more neurons (e.g., astrocyte-converted neurons and/or non-converted neurons) present within the brain (e.g., striatum) of a mammal (e.g., a human having Huntington's disease) can be used to reduce the presence of huntingtin protein having more than 11 consecutive glutamine residues within the brain. For example, gene therapy components can be designed to edit an Htt allele such that the edited Htt allele contains less than 36 CAG repeats and/or such that the edited Htt allele is unable to express a huntingtin polypeptide having more than 11 consecutive glutamine residues.

GABAergic MSNs within the striatum die or degenerate during Huntington's disease progression. As described herein, delivering nucleic acid designed to express a NeuroD1 polypeptide and nucleic acid designed to express a Dlx2 polypeptide to striatal astrocytes within a mammal's brain can convert the striatal astrocytes into GABAergic MSNs within the mammal's brain. The astrocyte-converted neurons can send out long-range nerve projections and strengthen GABAergic outputs from the striatum to the globus pallidus (GP) and substantia nigra pars reticulata (SNr) in the brain, and can result in fewer nuclear HTT polypeptide inclusions (e.g., aggregates of HTT polypeptides having a polyglutamine expansion) as compared to preexisting neurons in the brain. The in vivo regeneration of GABAergic neurons in the striatum can reduce striatum atrophy, improve motor functions, and increase the survival rate of Huntington's disease patients.

Having the ability to form new MSNs within the striatum of a living mammal's brain using the methods and materials described herein can allow clinicians and patients (e.g., Huntington's disease patients) to create a brain architecture that more closely resembles the architecture of a healthy brain when compared to the architecture of an untreated Huntington's disease patient's brain following the significant death or degeneration of GABAergic MSNs. In some cases, having the ability to replenish GABAergic MSNs within the striatum that die or degenerate during Huntington's disease progression using the methods and materials described herein can allow clinicians and patients to slow, delay, or reverse Huntington's disease progression. For example, the in vivo generated neurons (e.g., in vivo generated GABAergic MSNs) can rescue motor function deficits and extend life expectancy in Huntington's disease patients.

In general, one aspect of this document features a method for treating a mammal having Huntington's disease. The method comprises (or consists essentially of or consists of) (a) administering, to glial cells within a striatum of the mammal, nucleic acid encoding a NeuroD1 polypeptide and nucleic acid encoding a Dlx2 polypeptide, wherein the NeuroD1 polypeptide and the Dlx2 polypeptide are expressed by the glial cells, and wherein the glial cells form GABAergic neurons within the striatum; and (b) administering, to glial cells, neurons, or both within a brain (e.g., within the striatum) of the mammal, gene therapy components comprising (i) a nuclease or nucleic acid encoding the nuclease, (ii) a targeting nucleic acid sequence complementary to at least a portion of one or both Htt genes, and (iii) a donor nucleic acid comprising at least a fragment of a donor Htt gene comprising a CAG repeat region, wherein the CAG repeat region comprises less than 36 CAG repeats, wherein the donor nucleic acid replaces a sequence of one or both Htt genes present in glial cells, neurons, or both. The mammal can be a human. The glial cells of step (a) can be astrocytes. The GABAergic neurons can be DARPP32-positive. The GABAergic neurons can comprise axonal projections that extend out of the striatum. The axonal projections can extend into the globus pallidus (GP) of the mammal. The axonal projections can extend into the substantia nigra pars reticulata (SNr) of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The nuclease is a CRISPR-associated (Cas) nuclease, and the targeting nucleic acid sequence can be a guide RNA (gRNA) (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a transcription activator-like (TAL) effector DNA-binding domain. The administration of the nucleic acid encoding a NeuroD1 polypeptide and the nucleic acid encoding a Dlx2 polypeptide or the administration of the gene therapy components can comprise a direct injection into the striatum. The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for treating a mammal having Huntington's disease, wherein the mammal is heterozygous for an Htt allele having more than 36 CAG repeats. The method comprises (or consists essentially of or consists of) (a) administering, to glial cells within a striatum of the mammal, nucleic acid encoding a NeuroD1 polypeptide and nucleic acid encoding a Dlx2 polypeptide, wherein the NeuroD1 polypeptide and the Dlx2 polypeptide are expressed by the glial cells, and wherein the glial cells form GABAergic neurons within the striatum; and (b) administering, to glial cells, neurons, or both within a brain (e.g., within the striatum) of the mammal, a composition comprising (i) a nuclease or nucleic acid encoding the nuclease and (ii) a targeting nucleic acid sequence complementary to at least a portion of the Htt allele, wherein the composition edits the Htt allele of glial cells, neurons, or both to form an edited Htt allele, and wherein the edited Htt allele is unable to express a polypeptide comprising more than 11 consecutive glutamine residues. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for improving a motor function in a mammal having Huntington's disease. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering gene therapy components to glial cells, neurons, or both within a brain (e.g., within the striatum) of the mammal, wherein the gene therapy components reduce the number of CAG repeats in one or both Htt genes present in glial cells, neurons, or both to less than 36 CAG repeats. The motor function can be selected from the group consisting of tremors and seizures. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The gene therapy components can comprise (i) a nuclease or nucleic acid encoding the nuclease, (ii) a targeting nucleic acid sequence complementary to at least a portion of one or both Htt genes, and (iii) a donor nucleic acid comprising at least a fragment of a donor Htt gene comprising less than 36 CAG repeats. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for improving a motor function in a mammal having Huntington's disease, wherein the mammal is heterozygous for an Htt allele having more than 36 CAG repeats. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering, to glial cells, neurons, or both within a brain (e.g., within the striatum) of the mammal, a composition comprising (i) a nuclease or nucleic acid encoding the nuclease and (ii) a targeting nucleic acid sequence complementary to at least a portion of the Htt allele, wherein the composition edits the Htt allele of glial cells, neurons, or both to form an edited Htt allele, and wherein the edited Htt allele is unable to express a polypeptide comprising more than 11 consecutive glutamine residues. The motor function can be selected from the group consisting of tremors and seizures. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for improving life expectancy of a mammal having Huntington's disease. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering gene therapy components to glial cells, neurons, or both within a brain (e.g., within the striatum) of the mammal, wherein the gene therapy components reduce the number of CAG repeats in one or both Htt genes present in glial cells, neurons, or both to less than 36 CAG repeats. The life expectancy of the mammal can be extended by from about 10% to about 60%. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The gene therapy components can comprise (i) a nuclease or nucleic acid encoding the nuclease, (ii) a targeting nucleic acid sequence complementary to at least a portion of one or both Htt genes, and (iii) a donor nucleic acid comprising at least a fragment of a donor Htt gene comprising less than 36 CAG repeats. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for improving life expectancy of a mammal having Huntington's disease, wherein the mammal is heterozygous for an Htt allele having more than 36 CAG repeats. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering, to glial cells, neurons, or both within a brain (e.g., within the striatum) of the mammal, a composition comprising (i) a nuclease or nucleic acid encoding the nuclease and (ii) a targeting nucleic acid sequence complementary to at least a portion of the Htt allele, wherein the composition edits the Htt allele of glial cells, neurons, or both to form an edited Htt allele, and wherein the edited Htt allele is unable to express a polypeptide comprising more than 11 consecutive glutamine residues. The life expectancy of the mammal can be extended by from about 10% to about 60%. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for reducing striatum atrophy in a mammal having Huntington's disease. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering gene therapy components to glial cells, neurons, or both within a brain (e.g., within the striatum) of the mammal, wherein the gene therapy components reduce the number of CAG repeats in one or both Htt genes present in glial cells, neurons, or both to less than 36 CAG repeats. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The gene therapy components comprise (i) a nuclease or nucleic acid encoding the nuclease, and (ii) a targeting nucleic acid sequence complementary to at least a portion of one or both Htt genes. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for reducing striatum atrophy in a mammal having Huntington's disease, wherein the mammal is heterozygous for an Htt allele having more than 36 CAG repeats. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering, to glial cells, neurons, or both within a brain (e.g., within the striatum) of the mammal, a composition comprising (i) a nuclease or nucleic acid encoding the nuclease and (ii) a targeting nucleic acid sequence complementary to at least a portion of the Htt allele, wherein the composition edits the Htt allele of glial cells, neurons, or both to form an edited Htt allele, and wherein the edited Htt allele is unable to express a polypeptide comprising more than 11 consecutive glutamine residues. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for reducing nuclear HTT polypeptide inclusions in a mammal having Huntington's disease. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering gene therapy components to glial cells, neurons, or both within a brain (e.g., within the striatum) of the mammal, wherein the gene therapy components reduce the number of CAG repeats in one or both Htt genes present in glial cells, neurons, or both to less than 36 CAG repeats. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The gene therapy components can comprise (i) a nuclease or nucleic acid encoding the nuclease, (ii) a targeting nucleic acid sequence complementary to at least a portion of one or both Htt genes, and (iii) a donor nucleic acid comprising at least a fragment of a donor Htt gene comprising less than 36 CAG repeats. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intravenous, intrathecal, intracerebral, intraparenchymal, intranasal, or oral administration. The method can comprise, prior to the administering steps, identifying the mammal as having Huntington's disease.

In another aspect, this document features a method for reducing nuclear HTT polypeptide inclusions in a mammal having Huntington's disease, wherein the mammal is heterozygous for an Htt allele having more than 36 CAG repeats. The method comprises (or consists essentially of or consists of) (a) administering nucleic acid encoding a NeuroD1 polypeptide and nucleic acid encoding a Dlx2 polypeptide to glial cells within a striatum of the mammal, wherein the NeuroD1 polypeptide and the Dlx2 polypeptide are expressed by the glial cells, and wherein the glial cells form GABAergic neurons within the striatum; and (b) administering, to glial cells, neurons, or both within a brain (e.g., within the striatum) of the mammal, a composition comprising (i) a nuclease or nucleic acid encoding the nuclease and (ii) a targeting nucleic acid sequence complementary to at least a portion of the Htt allele, wherein the composition edits the Htt allele of glial cells, neurons, or both to form an edited Htt allele, and wherein the edited Htt allele is unable to express a polypeptide comprising more than 11 consecutive glutamine residues. The mammal can be a human. The glial cells of step (a) can be astrocytes. The GABAergic neurons can be DARPP32-positive. The GABAergic neurons can comprise axonal projections that extend out of the striatum. The axonal projections can extend into the GP of the mammal. The axonal projections can extend into the SNr of the mammal. The NeuroD1 polypeptide can be a human NeuroD1 polypeptide, or the Dlx2 polypeptide can be a human Dlx2 polypeptide. The nucleic acid encoding the NeuroD1 polypeptide or the nucleic acid encoding the Dlx2 polypeptide can be administered to the glial cells in the form of a viral vector. The viral vector can be an adeno-associated viral vector. The adeno-associated viral vector can be an adeno-associated serotype 2/5 viral vector. The nucleic acid encoding the NeuroD1 polypeptide and the nucleic acid encoding the Dlx2 polypeptide can be located on the same viral vector, and the viral vector can be administered to the glial cells of step (a). The nucleic acid encoding the NeuroD1 polypeptide and the nucleic acid encoding the Dlx2 polypeptide can be located on separate viral vectors, and each of the separate viral vectors can be administered to the glial cells of step (a). The nucleic acid encoding the NeuroD1 polypeptide or the nucleic acid encoding the Dlx2 polypeptide can be operably linked to a promoter sequence. The nuclease can be a Cas nuclease, and the targeting nucleic acid sequence can be a gRNA (or DNA encoding the gRNA). The nuclease can be selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and the targeting nucleic acid sequence can be a TAL effector DNA-binding domain. The administration of the nucleic acid encoding a NeuroD1 polypeptide and the nucleic acid encoding a Dlx2 polypeptide or the administration of the gene therapy components can comprise a direct injection into the brain (e.g., a direct injection into the striatum). The administration of the nucleic acid encoding a NeuroD1 polypeptide and the nucleic acid encoding a Dlx2 polypeptide or the administration of the gene therapy components can comprise an intraperitoneal, intramuscular, intrathecal, intracerebral, intraparenchymal, intravenous, intranasal, or oral administration. The method can comprise, prior to the administering steps, identifying the mammal as having Huntington's disease.

Unless otherwise defined, all technical and scientific terms used herein have the same meaning as commonly understood by one of ordinary skill in the art to which this invention pertains, as exemplified by various art-specific dictionaries. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and drawings, and from the claims.

### DESCRIPTION OF DRAWINGS

FIGS. 1A-1D. Exemplary engineered AAV2/5 Cre-FLEx system infects striatal astrocytes specifically in the adult mouse brain. FIG. 1A. Schematic diagram of engineered AAV2/5 constructs (GFAP::Cre and FLEx-CAG::mCherry-P2A-mCherry) used to target astrocytes specifically with GFAP promoter-controlled expression of Cre recombinase, which in turn will activate the expression of mCherry. FIG. 1B. Cre recombinase (which stained red) was detected specifically in GFAP positive astrocytes (which stained green) at 7 days post viral injection (dpi) of AAV2/5-GFAP::Cre. White arrowheads indicate astrocytes with Cre expression. Scale bar: 50 μm. FIG. 1C. Tiled confocal image of the striatum after control AAV mCherry injection (top left) (30 dpi), and the overlaid images of mCherry with a variety of glial markers or neuronal marker (NeuN). 510013, GFAP and glutamine synthetase (GS) are markers for astrocytes; Olig2 for oligodendrocytes; NG2 for NG2 expressing cells; and Iba1 for microglia. Arrowheads indicate some colocalized cells. Scale bar: 0.5 mm for the top tiled low magnification images, and 50 μm for the high magnification images. FIG. 1D. Percentage of mCherry positive cells in colocalization with different cell markers in the striatum. Note that the majority of control mCherry virus-infected cells were astrocytes. Data are shown as mean±SEM.

FIGS. 2A-2G. In vivo conversion of striatal astrocytes into GABAergic neurons in WT mouse brain. FIG. 2A. Co-expression of NeuroD1 (which stained green) and Dlx2 (which stained blue) together with mCherry (which stained red, NeuroD1-p2A-mCherry and Dlx2-P2A-mCherry) in AAV infected striatal astrocytes (GFAP, which stained cyan) at 7 dpi. FIG. 2B. At 30 dpi, NeuroD1 (which stained green) and Dlx2 (which stained blue) co-expressed cells became NeuN positive neurons (which stained cyan). Scale bar for a and b: 20 μm. FIG. 2C. Summarized data showing coexpression of NeuroD1 and Dlx2 in striatal astrocytes at 7 dpi, which mostly converted into NeuN positive neurons by 30 dpi (n=8 mice for 7 dpi, n=9 mice for 30 dpi). FIG. 2D. Diagram illustrating the astrocyte-to-neuron conversion process induced by NeuroD1 and Dlx2 co-expression. FIG. 2E. Representative images illustrating the gradual morphological change from astrocytes to neurons over a time window of one month. Note that most mCherry positive cells were co-labeled with GFAP (which stained cyan) at early time points post AAV injection, but later lost GFAP signal and acquired NeuN signal (which stained green). Arrowheads indicate mCherry positive cells that are co-labeled with NeuN. Scale bar: 50 μm. FIG. 2F. Time course showing the cell identity (astrocyte vs neuron) among viral infected cells (mCherry positive cells) in the control group (mCherry positive alone, top graph) or NeuroD1+Dlx2 group (bottom graph). Most of the viral infected cells in the control group were astrocytes, whereas the NeuroD1+Dlx2-infected cells gradually shifted from mainly astrocytic population to a mixed population of astrocytes and neurons, and then to mostly neuronal population. FIG. 2G. Confocal images showing converted neurons co-stained with GAD67, GABA, DARPP32, and parvalbumin (PV) after ectopic expression of NeuroD1 and Dlx2 in striatal astrocytes (30 dpi). Arrowheads indicate co559 labeled cells. Scale bar: 20 μm. (h) Quantified data showing the composition of the astrocyte-converted neurons induced by NeuroD1 and Dlx2 in the striatum. Most of the converted neurons were GABAergic neurons (>80%) and a significant proportion were immunopositive for DARPP32 (55.7%). Data are shown as mean±SEM.

FIGS. 3A-3B. Ectopic expression of NeuroD1 and Dlx2 in AAV-infected cells. FIG. 3A. Co-staining of Dlx2, NeuroD1, mCherry, and NeuN at 7 days post AAV2/5 injection (7 dpi). No NeuroD1 or Dlx2 were detected in NeuN positive cells at 7 dpi. FIG. 3B. Co-staining of Dlx2, NeuroD1, mCherry, and GFAP at 30 days post AAV2/5 injection (30 dpi). NeuroD1 and Dlx2 were colocalized with mCherry, but not with GFAP. Scale bar: 20 μm. Quantification was shown in FIG. 2c.

FIG. 4. Time course of mCherry control virus infection in the striatum of WT mice. WT mice were injected with AAV2/5 GFAP::Cre+AAV2/5 CAG::mCherry-P2A-mCherry, and sacrificed at different time points (7, 11, 15, 21, and 30 dpi) for immunohistochemistry analyses. Most of the mCherry positive cells co-stained with GFAP but not NeuN, with only a few exception at 21 and 30 dpi (arrowhead). Scale bar: 50 μm. Quantification was shown in FIG. 2f.

FIGS. 5A-5C. Synergistic effect of NeuroD1 and Dlx2 in increasing the conversion efficiency in the striatum. FIG. 5A. WT mice were injected with different AAV2/5 and sacrificed at 30 dpi for immunostaining analysis to compare the conversion efficiency among different groups. Scale bar: 50 μm. FIGS. 5B and 5C. Quantified data showing that the NeuroD1+Dlx2 group has the highest conversion efficiency (FIG. 5B) and generates the greatest number of neurons (FIG. 5C). Data are shown as mean±SEM.

FIG. 6. Neuronal subtype characterization among the striatal astrocyte-converted neurons in the WT mouse striatum. The mouse brain sections were co-stained with different GABAergic subtype markers at 30 dpi. Few converted neurons were positive for somatostatin (SST), neuropeptide Y (NPY), or calretinin. Scale bar: 20 μm. Quantified data were shown in FIG. 2h.

FIGS. 7A-7G. Striatal neuron and astrocyte density in WT mouse brain after conversion. FIG. 7A. Confocal images showing the astrocytic marker S100β and neuronal marker NeuN at 30 days post AAV injection. Scale bar: 20 μm. FIGS. 7B-7D. High magnification confocal images showing dividing astrocytes at different stages found in NeuroD1+Dlx2 treated mouse brains, indicating astrocytic proliferation after conversion. FIGS. 7E-7G. Summary graphs showing neuronal density (FIG. 7E), astrocytic density (FIG. 7F), and the ratio of neuron/astrocyte (FIG. 7G) in control condition or after cell conversion (N+D), with no significant difference. Data are shown as mean±SD.

FIGS. 8A-8D. Striatal neuron and microglia density in WT mouse brain after cell conversion. FIG. 8A. Confocal images showing the microglial marker Iba1 and neuronal marker NeuN at 30 days post AAV injection. Scale bar: 20 μm. FIGS. 8B-8D. Summary graphs showing neuronal density (FIG. 8B), microglial density (FIG. 8C) and the ratio of neuron/microglia (FIG. 8D) not changed after cell conversion. Data are shown as mean±SD.

FIGS. 9A-9F. Converted neurons originate from astrocytes traced by GFAP::Cre 77.6 transgenic mice. FIGS. 9A and 9B. Experimental timeline (FIG. 9A) and schematic diagram (FIG. 9B) illustrating the use of GFAP::Cre reporter mice to investigate the astrocyte-to-neuron conversion process in the striatum induced by NeuroD1+Dlx2 (FLEx-NeuroD1-P2A-mCherry and FLEx-Dlx2-P2A-mCherry). FIG. 9C. Typical confocal images showing the mCherry positive cells (NeuroD1+Dlx2) co-stained with GFAP and NeuN at 7 dpi (left column), 28 dpi (middle column), and 56 dpi (right column). Scale bar: 20 μm. Insets show a typical cell with different markers. Scale bar: 4 μm. FIG. 9D. Confocal images of mCherry positive cells (NeuroD1+Dlx2) co-stained with S100β and NeuN at 7, 28, and 56 dpi. Scale bar: 20 μm. Inset scale bar: 4 μm. FIGS. 9E and 9F. Quantified data showing a gradual transition from astrocytes to neurons over the time course of 2 months in the GFAP::Cre mice after injection of NeuroD1 and Dlx2 viruses. Note that besides a decrease of astrocytes and an increase of neurons among NeuroD1 and Dlx2-infected cells, about 40% of the infected cells were caught at a transitional stage at 28 dpi, which showed neither GFAP signal nor NeuN signal. Also note that the time course of astrocyte-to-neuron conversion is slower in GFAP::Cre mice compared to that induced by GFAP::Cre AAV2/5, both in combination with AAV2/5 FLEx-NeuroD1-P2A-mCherry and FLEx-Dlx2-P2A-mCherry. Data are shown as mean±SEM.

FIGS. 10A-10C. Targeting striatal astrocytes for neuronal conversion in the GFAP::Cre77.6 transgenic mouse line. FIG. 10A. Confocal images showing the control AAV mCherry-infected cells in the striatum co-staining with different glial markers and neuronal marker at 58 dpi. Most of the mCherry positive cells were colocalized with astrocytic markers including S100β, GFAP, and glutamine synthetase (GS). Very few mCherry positive cells co-stained with Olig2, NG2, Iba1, or NeuN. Scale bar: 20 μm. FIG. 10B. Quantified data of FIG. 10A showing the percentage of the mCherry positive cells that co-stained with different markers. Over 95% of mCherry positive cells were positive for astrocyte markers in the striatum of GFAP::Cre 77.6 mouse line. FIG. 10C. In NeuroD1+Dlx2-treated striatum of the GFAP::Cre 77.6 mouse line, the majority of the astrocyte-converted neurons were immunopositive for DARPP32 (58 dpi). Scale bar: 20 μm. Data are shown as mean±SEM.

FIGS. 11A-11F. In vivo conversion of striatal astrocytes into GABAergic neurons in the R6/2 mouse brain. FIG. 11A. A low-magnification coronal section of the R6/2 mouse striatum injected with control mCherry AAV (left panel) or NeuroD1+Dlx2 AAV (right panel) at 30 dpi. Scale bar: 0.5 mm. FIG. 11B. Higher-magnification images of mCherry positive cells co-stained with S100β (which stained green) and NeuN (which stained cyan). Arrowheads indicate mCherry positive cells co-labeled with S100β in the control group (top row), but in NeuroD1+Dlx2 group became co-labeled with NeuN (bottom row). Scale bar: 20 μm. FIG. 11C. Summary of data showing that by 30 dpi, the majority of mCherry positive cells in the control group were S100β positive astrocytes, while in the NeuroD1+Dlx2 group most of the mCherry positive cells were converted into NeuN positive neurons. Data are shown as mean±SEM. FIG. 11D. Most of the striatal astrocyte-converted neurons in the R6/2 mice were immunopositive for GAD67 and GABA. Scale bar: 20 μm. FIG. 11E. Many of the converted neurons were co-stained by DARPP32 and a few also co-stained with parvalbumin (PV). Scale bar: 20 μm. FIG. 11F. Quantified data showing that >80% of the converted neurons in the striatum of R6/2 mice were immunopositive for GAD67 and GABA, with a significant proportion also immunopositive for DARPP32 (56.6%) and a smaller percentage being PV positive (8.4%), but very few other GABAergic sub-types.

FIG. 12. Subtype characterization of converted neurons in the R6/2 mouse striatum. Among the striatal astrocyte-converted neurons following NeuroD1+Dlx2 treatment in the R6/2 mouse striatum, only a few of the converted neurons were immunopositive for somatostatin (SST), neuropeptide Y (NPY), or calretinin. Scale bar: 20 μm. Quantification was shown in FIG. 11f.

FIGS. 13A-G. Striatal neuron and astrocyte density in R6/2 mouse brains after cell conversion. FIG. 13A. Typical confocal images of astrocytes, AAV-infected cells, and neurons in R6/2 mouse striatum at 30 days after viral injection. Scale bar: 20 μm. FIGS. 13B-13D. High magnification confocal images showing different stages of dividing astrocytes in R6/2 mouse striatum after NeuroD1+Dlx2 treatment, indicating astrocytic proliferation after conversion. FIGS. 13E-13G. Summary graphs illustrating neuronal density (FIG. 13E), astrocytic density (FIG. 13F), and the ratio of neuron/astrocyte (FIG. 13G) in the R6/2 mouse striatum without (Ctrl) or with cell conversion (N+D). Data are shown as mean±SD.

FIGS. 14A-14C. Cell conversion triggers proliferation of striatal astrocytes in R6/2 mouse brains. FIG. 14A. Tiled low magnification confocal images of Ki67 immunostaining showing many proliferating cells detected in the NeuroD1+Dlx2-treated R6/2 mouse striatum, but very few in the striatum of control AAV-treated R6/2 mice. Scale bar: 100 μm. FIG. 14B. High magnification confocal images showing proliferating astrocytes (arrowheads) in R6/2 mouse striatum after NeuroD1+Dlx2 treatment. The arrow indicates a converted neuron (pseudo-color). Scale bar: 10 μm. FIG. 14C. Summary graph showing the number of proliferating astrocytes dramatically increased in NeuroD1+Dlx2-treated R6/2 striatum (30 dpi), suggesting that in vivo astrocyte-to-neuron conversion can significantly stimulate the proliferation of astrocytes to replenish themselves after astrocyte conversion. Data are shown as mean±SD.

FIGS. 15A-15D. Striatal neuron and microglia density in R6/2 mouse brains after cell conversion. FIG. 15A. Confocal images showing the microglial marker Iba1 and neuronal marker NeuN at 30 days post AAV injection. Scale bar: 20 μm.

FIGS. 15B-15D. Summary graphs showing neuronal density (FIG. 15B), microglial density (FIG. 15C), and the ratio of neuron/microglia (FIG. 15D) in control and NeuroD1+Dlx2 group. Data are shown as mean±SD.

FIGS. 16A-16R. Functional characterization of the striatal astrocyte-converted neurons in the R6/2 mouse brain slices. FIG. 16A. Phase and fluorescent images of a native neuron (mCherry negative, top row) and a converted neuron (mCherry positive, bottom row). Scale bar: 10 μm. FIG. 16B. Representative traces of Na positive K positive currents recorded in native (black) and converted neurons (which stained red). FIG. 16C. Repetitive action potentials (AP) evoked by step-wise current injections. Note a significant delay to the initial action potential firing upon depolarization stimulation in both native and converted neurons. Such delayed firing is a typical MSN electrophysiological property. FIGS. 16D and 16E. Typical traces of sEPSCs and sIPSCs recorded from native (top row) and converted neurons (bottom row). FIGS. 16F and 16G. I-V plot of Na positive K positive currents recorded from striatal neurons in the viral-injected R6/2 mice and non-treated WT mice. The Na positive currents in both converted and non-converted striatal neurons in the R6/2 mice were smaller than that recorded from the striatal neurons in the WT mice. The K positive current in converted neurons is significantly larger than that in non-converted neurons in the R6/2 mouse striatum (unpaired Student's t-test). *p<0.05, **p<0.01. Data are shown as mean±SEM. FIG. 16H-16M. Summary graphs in scatter-plot showing similar electrical properties among the converted and non-converted neurons in the R6/2 mice, together with the wild-type neurons: input resistance (FIG. 16H), capacitance (FIG. 16I), resting membrane potential (FIG. 16J), AP threshold (FIG. 16K), AP amplitude (FIG. 16L), and AP frequency (FIG. 16M). There were no significantly differences between the converted and non-converted neurons in the R6/2 mice, but neurons from R6/2 mice showed some differences from the wild-type neurons. One-way ANOVA with Bonferroni's post hoc test. FIG. 16N-16Q. Summary graphs in scatter-plot showing similar synaptic inputs among the wild-type neurons and the converted neurons and non-converted neurons in the R6/2 mice: sEPSC frequency (FIG. 16N), sEPSC amplitude (FIG. 16O), sIPSC frequency (FIG. 16P), and sIPSC (FIG. 16Q). The p value is >0.4 for all groups, one-way ANOVA with Bonferroni's post hoc test. FIG. 16R. Pie chart showing the percentage of neurons with different firing pattern among the converted neurons.

FIGS. 17A-17D. Typical electrophysiological traces recorded from striatal neurons in the wild type mice. FIG. 17A. Representative traces showing Na positive K positive currents recorded from striatal neurons in the wild type mouse. FIG. 17B. Typical traces of action potentials recorded from WT striatal neurons. FIGS. 17C and 17D. Typical traces of spontaneous EPSCs (FIG. 17C) and spontaneous IPSCs (FIG. 17D) recorded from WT striatal neurons.

FIGS. 18A-18G. Axonal projections of the striatal astrocyte-converted neurons in the R6/2 mouse brain. FIG. 18A. A sagittal view of a R6/2 mouse brain section immunostained for vGAT (which stained green) and tyrosine hydroxylase (TH, which stained cyan). TH positive cell bodies were present in the substantia nigra (above the SNr) and dense TH innervation was observed in the striatum. Inset shows the mCherry channel only to illustrate the axonal projections from the striatum to the GP and SNr. Scale bar: 1 mm. FIG. 18B. High-resolution images showing mCherry positive puncta co-stained with vGAT (arrowhead) in GP and SNr (38 dpi). Scale bar: 2 μm. FIG. 18C. Quantified data showing vGAT intensity in the GP and SNr significantly enhanced in NeuroD1+Dlx2 treated R6/2 mouse brains. FIG. 18D. Experimental design of CTB retrograde tracing of converted neurons in the R6/2 mouse brain. Mice were sacrificed for immunohistochemistry analysis at 7 days after CTB injection. FIG. 18E. Retrograde tracing of striatal astrocyte-converted neurons by injecting CTB into the GP at 21 or 30 days after AAV2/5 NeuroD1+Dlx2 injection. Few CTB (which stained green)-labeled converted neurons (which stained red) were detected in the striatum at 21 dpi group (arrowhead), but many more CTB-labeled converted neurons were observed at 30 dpi group (arrowheads). FIG. 18F. CTB injection into the SNr to trace striatal astrocyte-converted neurons. Even fewer converted neurons were labeled by CTB at 21 dpi group, but CTB labeling was clearly identified among the converted neurons in the striatum at 30 dpi group (arrowheads). Note that, in both GP (FIG. 18E) and SNr (FIG. 18F), many non-converted preexisting neurons were retrograde labeled by CTB, as expected. Scale bar for e and f: 20 μm. FIG. 18G. Bar graphs showing the percentage of CTB-labeled converted neurons in the R6/2 mouse striatum, which showed a significant increase from 21 dpi (black bars, immature neurons) to 30 dpi (gray bars, more mature neurons). *p<0.05, **p<0.01, unpaired Student's t-test. Data are shown as box plot (boxes, 25-75%; whiskers, 10-90%; lines, median).

FIGS. 19A and 19B. Sagittal view of R6/2 mouse brain showing axonal projection from newly converted neurons post NeuroD1+Dlx2 treatment. FIG. 19A. Tiled image showing sagittal view of R6/2 mouse brain at 38 days post viral injection of NeuroD1+Dlx2. mCherry positive converted neurons sent axonal projections to GP and SNr areas. FIG. 19B. Merged images showing the mCherry signal relative to other brain regions. Scale bar: 1 mm. This is enlarged view of FIG. 19a.

FIGS. 20A and 20B. Axonal projections of the striatal astrocyte-converted neurons in the R6/2 mouse brain. FIG. 20A. Sagittal tile image of an R6/2 mouse injected with control AAV mCherry (38 dpi). No mCherry positive signal detected in the GP or SNr. Scale bar: 1 mm. FIG. 20B. High-magnification images showing lack of mCherry positive signal in the GP and SNr after control virus injection (38 dpi), but a significant mCherry positive signal in both GP and SNr following NeuroD1+Dlx2 injection (38 dpi). Scale bar: 10 μm. The high-resolution images of mCherry and vGAT puncta were shown in FIG. 19b and quantified data were shown in FIG. 19c.

FIGS. 21A and 21B. Validating the sites of CTB injection. FIG. 21. A sagittal view of CTB injection in the GP. FIG. 21B. A sagittal view of CTB injection in the SNr. Mice were sacrificed at 7 days post CTB injection. Scale bar: 1 mm.

FIGS. 22A-22C. mHtt inclusions and striatum atrophy in non-surgery R6/2 mice. FIG. 22A. In non-surgery R6/2 mice, mHtt inclusions were mostly found in striatal neurons (NeuN) and less in astrocytes (S100β) (age of P60 and P90). Scale bar: 20 μm. FIG. 22B Quantified data of FIG. 22A. FIG. 22C. Niss1 staining of serial coronal sections of WT littermates and R6/2 mice without surgery (age of P90). Scale bar: 0.5 mm. The quantified data were shown in FIG. 23D.

FIGS. 23A-23D. Reducing striatum atrophy in the R6/2 mice after in vivo astrocyte-to-neuron conversion. FIG. 23A. Reduction of mHtt inclusions in the striatal astrocyte-converted neurons in the R6/2 mice. The mHtt aggregates (dots) were detected in most of the striatal neurons (NeuN), but some NeuroD1+Dlx2-converted neurons (pointed by arrows) showed no mHtt aggregates. Arrowheads indicate two converted neurons (mCherry positive) with mHtt inclusions. Scale bar: 10 μm. FIG. 23B. Assessing striatum atrophy by Niss1 staining of serial coronal sections of the R6/2 mouse brain, treated with control mCherry virus alone (top row) or with NeuroD1+Dlx2 AAV (bottom row). Scale bar: 0.5 mm. FIG. 23C. Quantified data showing that the percentage of neurons with mHtt inclusions in converted neurons was significantly lower compared to their neighboring native neurons or the striatal neurons in the control virus-treated group. FIG. 23D. Summary graphs of the relative striatum volume (normalized to the WT) among R6/2 mice (P90-97), R6/2 mice treated with control viruses, and R6/2 mice treated with NeuroD1+Dlx2 viruses. Striatal atrophy was clearly detected in the R6/2 mice (P90-97), but significantly rescued by NeuroD1+Dlx2 treatment. **p<0.01, ***p<0.001, One-way ANOVA with Bonferroni's post-hoc test. Data are shown as mean±SEM.

FIGS. 24A-24L. Functional improvement of the R6/2 mice following in vivo cell conversion. FIG. 24A. Representative footprint tracks among wild type littermates, R6/2 mice, R6/2 mice treated with control viruses or NeuroD1+Dlx2 viruses. Dashed lines indicate stride length (L) and width (W). FIGS. 24B and 24C. Quantified data of stride length (FIG. 24B) and width (FIG. 24C) among different groups. The stride length decreased in R6/2 mice, but partially rescued by NeuroD1+Dlx2 treatment (One-way ANOVA with Bonferroni's post-hoc test). FIG. 24D. Representative tracks showing locomotor activity in the open field test (20 min) among different groups. FIG. 24E. Quantified data showing the total travel distance reduced in R6/2 mice but significantly improved by NeuroD1+Dlx2 treatment (One-way ANOVA with Bonferroni's post-hoc test). **p<0.01, ***p<0.001. FIG. 24F. Average body weight of R6/2 mice at 7 days before surgery and 30 days after surgery (viral injection). NeuroD1+Dlx2-treated R6/2 mice showed less body weight loss than the control virus-treated mice at 30 dpi (*p<0.05, unpaired Student's t-test). Mouse number in each group is labeled in each bar. FIG. 24G. Typical clasping (top) and non-clasping (bottom) phenotype in the R6/2 mice. FIG. 24H. The percentage of mice showing clasping phenotype was decreased in NeuroD1+Dlx2-treated R6/2 mice (*p<0.05, 2-sided Pearson Chi-Square test). FIG. 24I. The average clasping score was also significantly reduced by NeuroD1+Dlx2 treatment (*p<0.05, unpaired Student's t-test). Mouse number in each group is labeled in the bar. FIG. 24J. The grip strength of R6/2 mice did not change following NeuroD1+Dlx2 treatment. FIG. 24K. Experimental diagram showing survival rate calculation from 7 days post-surgery to 38 days post-surgery (endpoint mouse age: P98). Mice that died between 7 dpi and 38 dpi were recorded. Behavioral tests were conducted between 30-37 dpi. FIG. 24L. Kaplan-Meier survival graph showing that 13 out of 29 R6/2 mice died in the control virus group, whereas only 2 out of 33 R6/2 mice died in the NeuroD1+Dlx2 treatment group (p<0.001, 2-sided Pearson Chi-Square test). Data are shown as box plot (boxes, 25-75%; whiskers, 10-90%; lines, median).

FIG. 25. A listing of an amino acid sequence of a human NeuroD1 polypeptide (SEQ ID NO:1).

FIG. 26. A listing of an amino acid sequence of a human Dlx2 polypeptide (SEQ ID NO:2).

### DETAILED DESCRIPTION

As used herein, the singular form “a,” “an,” and “the” include plural references unless.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

This document provides methods and materials for treating a mammal having Huntington's disease through regeneration of new functional neurons and reduction of mutant HTT toxicity. For example, nucleic acid encoding a NeuroD1 polypeptide and nucleic acid encoding a Dlx2 polypeptide can be used to convert glial cells (e.g., reactive astrocytes) within the brain (e.g., striatum) into GABAergic neurons (e.g., GABAergic MSNs) that are functionally integrated into the brain of a living mammal (e.g., a human) with Huntington's disease. Forming GABAergic neurons as described herein can include converting glial cells (e.g., astrocytes) within the brain into GABAergic neurons (e.g., astrocyte-converted neurons) that can be functionally integrated into the brain of a living mammal. In addition, one or more gene therapy components (e.g., a nuclease, a targeting sequence such as antisense oligonucleotides or guide RNAs, and/or a donor nucleic acid) designed to modify one or both Htt alleles (or its transcribed HTT RNAs or translated HTT polypeptides) present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can be used as described herein to reduce the amount of huntingtin protein having more than 11 consecutive glutamine residues within the brain. For example, gene therapy components can be designed to edit an Htt allele within glial cells and/or neurons in the striatum such that the edited Htt allele contains less than 36 CAG repeats and/or such that the edited Htt allele is unable to express a huntingtin polypeptide having more than 11 consecutive glutamine residues. In an aspect, the method and materials for treating a mammal having Huntington's disease (e.g., regeneration of new functional neurons and editing of an Htt allele) in combination has a synergistic effect on treating Huntington's disease symptoms, and/or improving outcomes and life expectancy.

Any appropriate mammal can be treated as described herein. For example, mammals including, without limitation, humans, monkeys, dogs, cats, cows, horses, pigs, rats, and mice, can be treated as described herein to generate GABAergic neurons and/or edit one or more Htt alleles in the brain of a living mammal. In some cases, a mammal is a male. In some cases, a mammal is a female. In some cases, a mammal is gender neutral. In some cases, a mammal is a premature newborn. In some cases, a premature newborn is born before 36 weeks gestation. In some cases, a mammal is a term newborn. In some cases, a term newborn is below about 2 months old. In some cases, a mammal is a neonate. In some, a neonate is below about 1 month old. In some cases, a mammal is an infant. In some cases, an infant is between 2 months and 24 months old. In some cases, an infant is between 2 months and 3 months, between 2 months and 4 months, between 2 months and 5 months, between 3 months and 4 months, between 3 months and 5 months, between 3 months and 6 months, between 4 months and 5 months, between 4 months and 6 months, between 4 months and 7 months, between 5 months and 6 months, between 5 months and 7 months, between 5 months and 8 months, between 6 months and 7 months, between 6 months and 8 months, between 6 months and 9 months, between 7 months and 8 months, between 7 months and 9 months, between 7 months and 10 months, between 8 months and 9 months, between 8 months and 10 months, between 8 months and 11 months, between 9 months and 10 months, between 9 months and 11 months, between 9 months and 12 months, between 10 months and 11 months, between 10 months and 12 months, between 10 months and 13 months, between 11 months and 12 months, between 11 months and 13 months, between 11 months and 14 months, between 12 months and 13 months, between 12 months and 14 months, between 12 months and 15 months, between 13 months and 14 months, between 13 months and 15 months, between 13 months and 16 months, between 14 months and 15 months, between 14 months and 16 months, between 14 months and 17 months, between 15 months and 16 months, between 15 months and 17 months, between 15 months and 18 months, between 16 months and 17 months, between 16 months and 18 months, between 16 months and 19 months, between 17 months and 18 months, between 17 months and 19 months, between 17 months and 20 months, between 18 months and 19 months, between 18 months and 20 months, between 18 months and 21 months, between 19 months and 20 months, between 19 months and 21 months, between 19 months and 22 months, between 20 months and 21 months, between 20 months and 22 months, between 20 months and 23 months, between 21 months and 22 months, between 21 months and 23 months, between 21 months and 24 months, between 22 months and 23 months, between 22 months and 24 months, and between 23 months and 24 months old. In some cases, a mammal is a toddler. In some cases, a toddler is between 1 year and 4 years old. In some cases, a toddler is between 1 year and 2 years, between 1 year and 3 years, between 1 year and 4 years, between 2 years and 3 years, between 2 years and 4 years, and between 3 years and 4 years old. In some cases, a mammal is a young child. In some cases, a young child is between 2 years and 5 years old. In some cases, a young child is between 2 years and 3 years, between 2 years and 4 years, between 2 years and 5 years, between 3 years and 4 years, between 3 years and 5 years, and between 4 years and 5 years old. In some cases, a mammal is a child. In some cases, a child is between 6 years and 12 years old. In some cases, a child is between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, and between 11 years and 12 years old. In some cases, a mammal is an adolescent. In some cases, an adolescent is between 13 years and 19 years old. In one aspect, an adolescent is between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, between 16 years and 19 years, between 17 years and 18 years, between 17 years and 19 years, and between 18 years and 19 years old. In some cases, a mammal is a pediatric subject. In some cases, a pediatric subject between 1 day and 18 years old. In some cases, a pediatric subject is between 1 day and 1 year, between 1 day and 2 years, between 1 day and 3 years, between 1 year and 2 years, between 1 year and 3 years, between 1 year and 4 years, between 2 years and 3 years, between 2 years and 4 years, between 2 years and 5 years, between 3 years and 4 years, between 3 years and 5 years, between 3 years and 6 years, between 4 years and 5 years, between 4 years and 6 years, between 4 years and 7 years, between 5 years and 6 years, between 5 years and 7 years, between 5 years and 8 years, between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, between 10 years and 13 years, between 11 years and 12 years, between 11 years and 13 years, between 11 years and 14 years, between 12 years and 13 years, between 12 years and 14 years, between 12 years and 15 years, between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, and between 17 years and 18 years old. In some cases, a mammal is a geriatric mammal. In some cases, a geriatric mammal is between 65 years and 95 or more years old. In some cases, a geriatric mammal is between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In some cases, a mammal is an adult. In some cases, an adult mammal is between 20 years and 95 or more years old. In some cases, an adult mammal is between 20 years and 25 years, between 20 years and 30 years, between 20 years and 35 years, between 25 years and 30 years, between 25 years and 35 years, between 25 years and 40 years, between 30 years and 35 years, between 30 years and 40 years, between 30 years and 45 years, between 35 years and 40 years, between 35 years and 45 years, between 35 years and 50 years, between 40 years and 45 years, between 40 years and 50 years, between 40 years and 55 years, between 45 years and 50 years, between 45 years and 55 years, between 45 years and 60 years, between 50 years and 55 years, between 50 years and 60 years, between 50 years and 65 years, between 55 years and 60 years, between 55 years and 65 years, between 55 years and 70 years, between 60 years and 65 years, between 60 years and 70 years, between 60 years and 75 years, between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In some cases, a mammal is between 1 year and 5 years, between 2 years and 10 years, between 3 years and 18 years, between 21 years and 50 years, between 21 years and 40 years, between 21 years and 30 years, between 50 years and 90 years, between 60 years and 90 years, between 70 years and 90 years, between 60 years and 80 years, or between 65 years and 75 years old. In some cases, a mammal is a young old mammal (65 to 74 years old). In some cases, a mammal is a middle old mammal (75 to 84 years old). In one aspect, a subject in need thereof is an old mammal (>85 years old). In some cases, a mammal (e.g., a human) having Huntington's disease can be treated as described herein to generate GABAergic neurons and/or edit one or more Htt alleles in a Huntington's disease patient's brain. A mammal can be identified as having Huntington's disease using any appropriate Huntington's disease diagnostic technique. For example, non-limiting examples include a genetic screen of the Huntingtin gene, assessment of motor function deficits, assessment of memory deficits, phycological conditions assessment to include but not limited to depression and anxiety, magnetic resonance imaging (MRI), functional magnetic resonance imaging (fMRI), and positron emission tomography (PET) scan can be performed to diagnose a human as having Huntington's disease.

As described herein, a mammal (e.g., a human) having Huntington's disease can be treated by administering nucleic acid designed to express a NeuroD1 polypeptide and nucleic acid designed to express a Dlx2 polypeptide to glial cells (e.g., astrocytes) within the mammal's brain (e.g., striatum) in a manner that triggers the glial cells to form functional and integrated GABAergic neurons, and by administering one or more gene therapy components (e.g., a nuclease, a targeting sequence, and a donor nucleic acid) designed to modify the number of CAG repeats present in one or both Htt genes within the mammal's brain (e.g., striatum).

Examples of NeuroD1 polypeptides include, without limitation, those polypeptides having the amino acid sequence set forth in GenBank® accession number NP_002491 (GI number 121114306). A NeuroD1 polypeptide can be encoded by a nucleic acid sequence as set forth in GenBank® accession number NM_002500 (GI number 323462174). Examples of Dlx2 polypeptides include, without limitation, those polypeptides having the amino acid sequence set forth in GenBank® accession number NP_004396 (GI number 4758168). A Dlx2 polypeptide can be encoded by a nucleic acid sequence as set forth in GenBank® accession number NM_004405 (GI number 84043958). In some cases, nucleic acid designed to express a NeuroD1 polypeptide and/or nucleic acid designed to express a Dlx2 polypeptide can be as described elsewhere (see, e.g., WO 2017/143207).

Any appropriate method can be used to deliver nucleic acid designed to express a NeuroD1 polypeptide and nucleic acid designed to express a Dlx2 polypeptide to glial cells within the brain of a living mammal. For example, nucleic acid encoding a NeuroD1 polypeptide and nucleic acid encoding a Dlx2 polypeptide can be administered to a mammal using one or more vectors such as viral vectors. In some cases, separate vectors (e.g., one vector for nucleic acid encoding a NeuroD1 polypeptide, and one vector for nucleic acid encoding a Dlx2 polypeptide) can be used to deliver the nucleic acids to glial cells. In some cases, a single vector containing both nucleic acid encoding a NeuroD1 polypeptide and nucleic acid encoding a Dlx2 polypeptide can be used to deliver the nucleic acids to glial cells.

In some cases, vectors for administering nucleic acid (e.g., nucleic acid designed to express a NeuroD1 polypeptide and nucleic acid designed to express a Dlx2 polypeptide) to glial cells can be used for transient expression of a NeuroD1 polypeptide and/or a Dlx2 polypeptide.

In some cases, vectors for administering nucleic acid (e.g., nucleic acid designed to express a NeuroD1 polypeptide and nucleic acid designed to express a Dlx2 polypeptide) to glial cells can be used for stable expression of a NeuroD1 polypeptide and/or a Dlx2 polypeptide. In cases where a vector for administering nucleic acid can be used for stable expression of a NeuroD1 polypeptide and a Dlx2 polypeptide, the vector can be engineered to integrate nucleic acid designed to express a NeuroD1 polypeptide and/or nucleic acid designed to express a Dlx2 polypeptide into the genome of a glial cell. In some cases, vector is engineered to integrate nucleic acid designed to express a NeuroD1 polypeptide and/or nucleic acid designed to express a Dlx2 polypeptide into the genome of a glial cell, any appropriate method can be used to integrate that nucleic acid into the genome of a glial cell. For example, gene therapy techniques can be used to integrate nucleic acid designed to express a NeuroD1 polypeptide and/or nucleic acid designed to express a Dlx2 polypeptide into the genome of a glial cell.

Vectors for administering nucleic acids (e.g., nucleic acid encoding a NeuroD1 polypeptide and nucleic acid encoding a Dlx2 polypeptide) to glial cells can be prepared using standard materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). Virus-based nucleic acid delivery vectors are typically derived from animal viruses, such as adenoviruses, adeno-associated viruses (AAVs), retroviruses, lentiviruses, vaccinia viruses, herpes viruses, and papilloma viruses. In some cases, nucleic acid encoding a NeuroD1 polypeptide and nucleic acid encoding a Dlx2 polypeptide can be delivered to glial cells using adeno-associated virus vectors (e.g., an AAV serotype 1 viral vector, an AAV serotype 2 viral vector, an AAV serotype 3 viral vector, an AAV serotype 4 viral vector, an AAV serotype 5 viral vector, an AAV serotype 6 viral vector, an AAV serotype 7 viral vector, an AAV serotype 8 viral vector, an AAV serotype 9 viral vector, an AAV serotype 10 viral vector, an AAV serotype 11 viral vector, an AAV serotype 12 viral vector, or a recombinant AAV serotype viral vector such as an AAV serotype 2/5 viral vector), lentiviral vectors, retroviral vectors, adenoviral vectors, herpes simplex virus vectors, or poxvirus vector.

In addition to nucleic acid encoding a NeuroD1 polypeptide and/or nucleic acid encoding a Dlx2 polypeptide, a viral vector can contain regulatory elements operably linked to the nucleic acid encoding a NeuroD1 polypeptide and/or a Dlx2 polypeptide. Such regulatory elements can include promoter sequences, enhancer sequences, response elements, signal peptides, internal ribosome entry sequences, polyadenylation signals, terminators, or inducible elements that modulate expression (e.g., transcription or translation) of a nucleic acid. The choice of element(s) that may be included in a viral vector depends on several factors, including, without limitation, inducibility, targeting, and the level of expression desired. For example, a promoter can be included in a viral vector to facilitate transcription of a nucleic acid encoding a NeuroD1 polypeptide and/or a Dlx2 polypeptide. A promoter can be constitutive or inducible (e.g., in the presence of tetracycline), and can affect the expression of a nucleic acid encoding a polypeptide in a general or tissue-specific manner. Examples of tissue-specific promoters that can be used to drive expression of a NeuroD1 polypeptide and/or a Dlx2 polypeptide in glial cells include, without limitation, GFAP, NG2, Olig2, CAG, EF1a, Aldh1L1, and CMV promoters.

As used herein, “operably linked” refers to positioning of a regulatory element in a vector relative to a nucleic acid in such a way as to permit or facilitate expression of the encoded polypeptide. For example, a viral vector can contain a glial-specific GFAP promoter and nucleic acid encoding a NeuroD1 polypeptide or a Dlx2 polypeptide. In this case, the GFAP promoter is operably linked to a nucleic acid encoding a NeuroD1 polypeptide or a Dlx2 polypeptide such that it drives transcription in glial cells.

Nucleic acid encoding a NeuroD1 polypeptide and/or a Dlx2 polypeptide also can be administered to a mammal using non-viral vectors. Methods of using non-viral vectors for nucleic acid delivery are described elsewhere. See, for example, Gene Therapy Protocols (Methods in Molecular Medicine), edited by Jeffrey R. Morgan, Humana Press, Totowa, N.J. (2002). For example, nucleic acid encoding a NeuroD1 polypeptide and/or a Dlx2 polypeptide can be administered to a mammal by direct injection of nucleic acid molecules (e.g., plasmids) comprising nucleic acid encoding a NeuroD1 polypeptide and/or a Dlx2 polypeptide, or by administering nucleic acid molecules complexed with lipids, polymers, or nanospheres. In some cases, a genome editing technique such as CRISPR/Cas9-mediated gene editing can be used to activate endogenous NeuroD1 and/or Dlx2 gene expression.

Nucleic acid encoding a NeuroD1 polypeptide and/or a Dlx2 polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a NeuroD1 polypeptide and/or a Dlx2 polypeptide.

In some cases, NeuroD1 polypeptides and/or Dlx2 polypeptides can be administered in addition to or in place of nucleic acid designed to express a NeuroD1 polypeptide and/or nucleic acid designed to express a Dlx2 polypeptide. For example, NeuroD1 polypeptides and/or Dlx2 polypeptides can be administered to a mammal to trigger glial cells within the brain into forming GABAergic neurons that can be functionally integrated into the brain of the living mammal.

Nucleic acid designed to express a NeuroD1 polypeptide and nucleic acid designed to express a Dlx2 polypeptide (or NeuroD1 and/or Dlx2 polypeptides) can be delivered to glial cells within the brain (e.g., glial cells within the striatum) via direct intracranial injection, direct injection into the striatum, intraperitoneal administration, intranasal administration, intravenous administration, intrathecal administration, intracerebral administration, intraparenchymal administration, or oral delivery in nanoparticles and/or drug tablets, capsules, or pills.

As used herein, the term “AAV particle” refers to packaged capsid forms of the AAV virus that transmits its nucleic acid genome to cells.

In some cases, a composition comprising an AAV particle encoded by an AAV vector as provided herein is injected at a concentration between 10¹⁰ AAV particles/mL and 10¹⁴ AAV particles/mL. In some cases, a composition comprising an AAV particle encoded by an AAV vector as provided herein is injected at a concentration between 10¹⁰ AAV particles/mL and 10¹¹ AAV particles/mL, between 10¹⁰ AAV particles/mL and 10¹² AAV particles/mL, between 10¹⁰ AAV particles/mL and 10¹³ AAV particles/mL, between 10¹¹ AAV particles/mL and 10¹² AAV particles/mL, between 10¹¹ AAV particles/mL and 10¹³ AAV particles/mL, between 10¹¹ AAV particles/mL and 10¹⁴ AAV particles/mL, between 10¹² AAV particles/mL and 10¹³ AAV particles/mL, between 10¹² AAV particles/mL and 10¹⁴ AAV particles/mL, or between 10¹³ AAV particles/mL and 10¹⁴ AAV particles/mL. As described herein, nucleic acid designed to express a NeuroD1 polypeptide and nucleic acid designed to express a Dlx2 polypeptide (or NeuroD1 and/or Dlx2 polypeptides) can be administered to a mammal (e.g., a human) having Huntington's disease and used to treat the mammal. In some cases, nucleic acid designed to express a polypeptide having the amino acid sequence set forth in SEQ ID NO:1 and nucleic acid designed to express a polypeptide having the amino acid sequence set forth in SEQ ID NO:2 (or a polypeptide having the amino acid sequence set forth in SEQ ID NO:1 and/or a polypeptide having the amino acid sequence set forth in SEQ ID NO:2) can be administered to a mammal (e.g., a human) having Huntington's disease as described herein and used to treat the mammal. For example, a single adeno-associated viral vector can be designed to express a polypeptide having the amino acid sequence set forth in SEQ ID NO:1 and a polypeptide having the amino acid sequence set forth in SEQ ID NO:2, and that designed viral vector can be administered to a mammal (e.g., a human) having Huntington's disease to treat the mammal.

In some cases, a polypeptide containing the entire amino acid sequence set forth in SEQ ID NO:1, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. For example, nucleic acid designed to express a polypeptide containing the entire amino acid sequence set forth in SEQ ID NO:1 with one to ten amino acid additions, deletions, substitutions, or combinations thereof and nucleic acid designed to express a Dlx2 polypeptide (or the polypeptides themselves) can be designed and administered to a mammal (e.g., a human) having Huntington's disease to treat Huntington's disease.

In some cases, a polypeptide containing the entire amino acid sequence set forth in SEQ ID NO:2, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used. For example, nucleic acid designed to express a polypeptide containing the entire amino acid sequence set forth in SEQ ID NO:2 with one to ten amino acid additions, deletions, substitutions, or combinations thereof and nucleic acid designed to express a NeuroD1 polypeptide (or the polypeptides themselves) can be designed and administered to a mammal (e.g., a human) having Huntington's disease to treat Huntington's disease. In another example, nucleic acid designed to express a polypeptide containing the entire amino acid sequence set forth in SEQ ID NO:1 with one to ten amino acid additions, deletions, substitutions, or combinations thereof and nucleic acid designed to express a polypeptide containing the entire amino acid sequence set forth in SEQ ID NO:2 with one to ten amino acid additions, deletions, substitutions, or combinations thereof can be designed and administered to a mammal (e.g., a human) having Huntington's disease to treat Huntington's disease.

Any appropriate amino acid residue set forth in SEQ ID NO:1 and/or SEQ ID NO:2 can be deleted, and any appropriate amino acid residue (e.g., any of the 20 conventional amino acid residues or any other type of amino acid such as ornithine or citrulline) can be added to or substituted within the sequence set forth in SEQ ID NO:1 and/or SEQ ID NO:2. The majority of naturally occurring amino acids are L-amino acids, and naturally occurring polypeptides are largely comprised of L-amino acids. D-amino acids are the enantiomers of L-amino acids. In some cases, a polypeptide as provided herein can contain one or more D-amino acids. In some cases, a polypeptide can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for SEQ ID NO:1 and/or SEQ ID NO:2 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Further examples of conservative substitutions that can be made at any appropriate position within SEQ ID NO:1 and/or SEQ ID NO:2 are set forth in Table 1.

TABLE 1
Examples of conservative amino acid substitutions.

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some cases, polypeptides can be designed to include the amino acid sequence set forth in SEQ ID NO:1 or SEQ ID NO:2 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods disclosed herein.

In some cases, a polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:1, provided that it includes at least one difference (e.g., at least one amino acid addition, deletion, or substitution) with respect to SEQ ID NO:1, can be used. For example, nucleic acid designed to express a polypeptide containing an amino acid sequence with between 90% and 99% sequence identity to the amino acid sequence set forth in SEQ ID NO:1 and nucleic acid designed to express a Dlx2 polypeptide (or the polypeptides themselves) can be designed and administered to a mammal (e.g., a human) having Huntington's disease to treat Huntington's disease.

In some cases, a polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:2, provided that it includes at least one difference (e.g., at least one amino acid addition, deletion, or substitution) with respect to SEQ ID NO:2, can be used. For example, nucleic acid designed to express a polypeptide containing an amino acid sequence with between 90% and 99% sequence identity to the amino acid sequence set forth in SEQ ID NO:2 and nucleic acid designed to express a NeuroD1 polypeptide (or the polypeptides themselves) can be designed and administered to a mammal (e.g., a human) having Huntington's disease to treat Huntington's disease. In another example, nucleic acid designed to express a polypeptide containing an amino acid sequence with between 90% and 99% sequence identity to the amino acid sequence set forth in SEQ ID NO:1 and nucleic acid designed to express a polypeptide containing an amino acid sequence with between 90% and 99% sequence identity to the amino acid sequence set forth in SEQ ID NO:2 (or the polypeptides themselves) can be designed and administered to a mammal (e.g., a human) having Huntington's disease to treat Huntington's disease.

Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1 or SEQ ID NO:2) is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C: \B12seq c:\seq1.txt -j c:\seq2.txt -p blastn -o c:\output.txt -q -1-r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seq2.txt -p blastp -o c: \output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:1), followed by multiplying the resulting value by 100. For example, an amino acid sequence that has 340 matches when aligned with the sequence set forth in SEQ ID NO:1 is 95.5 percent identical to the sequence set forth in SEQ ID NO:1 (i.e., 340±356×100=95.5056). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

When generating a neuron within the brain of a living mammal (e.g., a human) with Huntington's disease as described herein (e.g., by triggering one or more astrocytes within the brain to form GABAergic MSNs), the generated neuron can be any appropriate type of neuron. In some cases, a neuron generated as described herein can resemble a PV positive neuron. In some cases, a neuron generated as described herein can be a MSN. In some cases, a neuron generated as described herein can be DARPP32-positive. In some cases, a neuron generated as described herein can have one or more axonal projections that can extend to a distant target (e.g., a target outside of the striatum) within the brain of a living mammal. For example, when a neuron generated as described herein has one or more axonal projections that extends to a distant target within the brain of a living mammal, the distant target can be as far as the original neuronal axons reached during brain development. In some cases, a newly generated neuron may follow the original axon pathways.

When a neuron generated as described herein has one or more axonal projections that can extend to a distant target (e.g., a target outside of the striatum) within the brain of a living mammal, the distant target can be any appropriate location within the brain of the mammal. Examples of distant targets within the brain of a living mammal to which one or more axonal projections from a neuron generated as described herein can extend include, without limitation, the SNr, the GP (e.g., the external GP), thalamus, hypothalamus, amygdala, and/or cortex within the brain of a living mammal.

Gene therapy components (e.g., gene editing components) designed to edit one or more Htt alleles within glial cells and/or neurons in the striatum as described herein can be any appropriate gene therapy components. In some cases, a gene editing component can be a nucleic acid (e.g., a targeting sequence and a donor nucleic acid). In some cases, a gene editing component can be polypeptide (e.g., a nuclease). In some cases, gene therapy components designed to modify one or more Htt alleles such that the edited or resulting Htt allele contains less than 36 CAG repeats and/or such that the edited or resulting Htt allele is unable to express a huntingtin polypeptide having more than 11 consecutive glutamine residues can be used in a gene therapy (e.g., gene replacement or gene editing) technique to treat the mammal. For example, a mammal (e.g., a mammal having Huntington's disease) can be treated by administering to the mammal a nuclease, a targeting sequence, and, optionally, a donor nucleic acid designed to modify one or both Htt genes present in one or more glial cells (e.g., astrocytes) and/or one or more neurons (e.g., astrocyte-converted neurons and/or non-converted neurons) within the mammal's brain (e.g., striatum). In some cases, a nuclease, a targeting sequence, and/or a donor nucleic acid designed to modify one or both Htt genes present in a mammal can be used to reduce the number of CAG repeats present in one or more glial cells (e.g., astrocytes) and/or one or more neurons (e.g., astrocyte-converted neurons and/or converted neurons) within the mammal's brain (e.g., striatum). For example, a nuclease, a targeting sequence, and/or a donor nucleic acid designed to modify the number of CAG repeats present in one or both Htt genes present in a mammal can be used to reduce the number of CAG repeats present in one or more glial cells (e.g., astrocytes) and/or one or more neurons (e.g., astrocyte-converted neurons and/or non-converted neurons) within the mammal's brain to less than 36 CAG repeats (e.g., 35, 34, 33, 32, 31, 30, 29, 28, 27, or fewer CAG repeats). For example, a nuclease, a targeting sequence, and a donor nucleic acid designed to modify the number of CAG repeats present in one or both Htt genes present in a mammal can be used to reduce the number of CAG repeats present in one or more glial cells (e.g., astrocytes) and/or one or more neurons (e.g., astrocyte-converted neurons and/or non-converted neurons) within the mammal's brain to a number of CAG repeats that is from about 27 CAG repeats to about 35 CAG repeats. In some cases, a modified Htt gene having less than 36 CAG repeats that is present in a mammal with Huntington's disease can encode a functional HTT polypeptide.

In some cases, a nuclease and a targeting sequence (with or without a donor nucleic acid) designed to modify one or both Htt genes (or its transcribed HTT RNAs or translated HTT polypeptides) present in one or more glial cells and/or one or more neurons within the striatum of a mammal can be used reduce or prevent expression of a huntingtin polypeptide having more than 11 consecutive glutamine residues by those glial cells and/or neurons. For example, a nuclease and a targeting sequence (and, optionally, a donor nucleic acid) designed to modify one or both Htt alleles can be used to create an edited or resulting Htt allele that is unable to express a huntingtin polypeptide having more than 11 consecutive glutamine residues. Examples of such edited or resulting Htt alleles include, without limitation, Htt alleles with an altered promotor or enhancer that results in lower expression of the encoded huntingtin polypeptide, Htt alleles with an altered promotor or enhancer that results in no expression of the encoded huntingtin polypeptide, Htt alleles with a stop codon present upstream of the CAG repeat region, Htt alleles lacking one or more exons (e.g., lacking the exon that encodes the CAG repeats), Htt alleles having a frame shift or a segment deletion in the Htt allele to reduce or prevent the HTT expression, and Htt alleles containing an added target sequence that directly reduces HTT RNAs or HTT polypeptides through direct or indirect binding.

A diploid mammal such as a human has two copies of each gene present in its genome. In some cases, a mammal having Huntington's disease can have more than 36 CAG repeats present in both copies of a Htt gene (e.g., can be homozygous for Huntington's disease) present in one or more neurons within the mammal's brain. In some cases, a mammal having Huntington's disease can have more than 36 CAG repeats present in one copy of a Htt gene (e.g., can be heterozygous for Huntington's disease) present in one or more neurons within the mammal's brain. When the methods and materials described herein include modifying one or more Htt alleles (e.g., modifying the number of CAG repeats present in a Htt gene) present in a mammal (e.g., a human) having Huntington's disease, one or both copies of the Htt gene present in a mammal can be modified. In cases where a mammal having Huntington's disease is homozygous for Huntington's disease, the methods and materials described herein can include modifying both copies of the Htt gene present in one or more neurons within the mammal's brain (e.g., striatum) that includes more than 36 CAG repeats. In cases where a mammal having Huntington's disease is heterozygous for Huntington's disease, the methods and materials described herein can include modifying only the copy of the Htt gene present in one or more neurons within the mammal's brain (e.g., striatum) that includes more than 36 CAG repeats. For example, clustered regularly interspaced short palindromic repeat (CRISPR)/CRISPR-associated (Cas) nuclease (CRISPR/Cas) techniques can be used to replace or edit an Htt allele having more than 36 CAG repeats such that the resulting allele has less than 36 CAG repeats and/or such that the resulting allele is unable to express a huntingtin polypeptide having more than 11 consecutive glutamine residues.

Any appropriate gene therapy technique can be used to modify an Htt allele present in one or more glial cells (e.g., astrocytes) and/or one or more neurons (e.g., astrocyte-converted neurons and/or non-converted neurons) within a mammal's brain (e.g., striatum). Examples of gene therapy techniques that can be used to modify one or both Htt alleles present in one or more glial cells (e.g., astrocytes) and/or one or more neurons (e.g., astrocyte-converted neurons and/or non-converted neurons) within a mammal's brain include, without limitation, gene replacement (e.g., using homologous recombination or homology-directed repair), gene editing, antisense oligonucleotides, and microRNAs.

In some cases, gene replacement can be used to modify one or both Htt alleles present in one or more glial cells (e.g., astrocytes) and/or one or more neurons (e.g., astrocyte-converted neurons and/or non-converted neurons) within the brain (e.g., striatum) of a mammal (e.g., a human having Huntington's disease). For example, donor nucleic acid including a fragment of an Htt gene that includes the CAG region and has less than 36 CAG repeats in that region can be introduced into one or more glial cells and/or neurons to replace the deleterious CAG region of one or both Htt alleles present in the glial cell(s) and/or neuron(s). In some cases, donor nucleic acid including a fragment of an Htt gene that includes the CAG region and has less than 36 CAG repeats in that region can be introduced into glial cells and/or neurons to integrate the donor nucleic acid into the genome of a glial cell and/or neuron such that, when integrated into the genome (e.g., integrated in-frame into one or both Htt genes present in the mammal), the nucleic acid can encode a functional HTT polypeptide.

In some cases, donor nucleic acid can be designed to encode a truncated huntingtin polypeptide that lacks the poly-glutamine region and the amino acid sequence downstream of the poly-glutamine region. For example, donor nucleic acid can be designed to include a stop codon upstream of the CAG repeat region.

Donor nucleic acid (e.g., donor nucleic acid including a fragment of an Htt gene that includes the CAG region and has less than 36 CAG repeats in that region) can be any appropriate form of nucleic acid. For example, donor nucleic acid including a fragment of an Htt gene that includes the CAG region and has less than 36 CAG repeats in that region can be a vector (e.g., a viral vector). Examples of vectors that can be used to as a gene replacement or gene editing vector for administering donor nucleic acid to glial cells and/or neurons can include, without limitation, viral vectors such as retroviral vectors, adenoviral vectors, adeno-associated viral vectors (e.g., dual AAV vectors or triple AAV vectors), lentiviral vectors, herpes viral vectors, and poxvirus vector. In some cases, donor nucleic acid described herein can be a lentiviral vector or an adenoviral vector.

In addition to a modified Htt allele sequence (e.g., a fragment of an Htt gene that includes the CAG region and has less than 36 CAG repeats in that region), donor nucleic acid can contain one or more elements (e.g., one or more targeting sequences that are complementary to at least a portion of the one or both Htt genes) for targeting the donor nucleic acid to one or both Htt genes present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease). In some cases, a targeting sequence can be a homology arm. For example, donor nucleic acid (e.g., donor nucleic acid including a fragment of an Htt gene that includes the CAG region and has less than 36 CAG repeats in that region) can have a region of homology (e.g., a homology arm) at each end (e.g., at the 3′ end and at the 5′ end) that can direct or further direct the donor nucleic acid to a Htt gene. In some cases, a homology arm at one end (e.g., a 3′ end) of donor nucleic acid can be homologous to a genomic region upstream of a Htt gene within a glial cell and/or a neuron, and a homology arm at the other end (e.g., a 5′ end) of donor nucleic acid can be homologous to a genomic region downstream of a Htt gene within a glial cell and/or a neuron. A homology arm can be any appropriate size. In some cases, a homology arm can be from about 100 nucleotides to about 2500 nucleotides in length. In some cases, a homology arm can be from about 100 nucleotides to about 2000 nucleotides. In some cases, a homology arm can be from about 100 nucleotides to about 1500 nucleotides. In some cases, a homology arm can be from about 100 nucleotides to about 1000 nucleotides. In some cases, a homology arm can be from about 100 nucleotides to about 500 nucleotides.

Donor nucleic acid (e.g., donor nucleic acid including a fragment of an Htt gene that includes the CAG region and has less than 36 CAG repeats in that region) can be introduced into one or more glial cells (e.g., astrocytes) and/or one or more neurons (e.g., astrocyte-converted neurons and/or non-converted neurons) present within the brain (e.g., striatum) of a mammal (e.g., a human having Huntington's disease) using any appropriate method. A method of introducing donor nucleic acid into one or more glial cells and/or one or more neurons present within the brain of a mammal can be a physical method. A method of introducing donor nucleic acid into one or more glial cells and/or one or more neurons present within the brain of a mammal can be a chemical method. A method of introducing donor nucleic acid into one or more glial cells and/or one or more neurons present within the brain of a mammal can be a biological method. A method of introducing donor nucleic acid (e.g., donor nucleic acid including a fragment of an Htt gene that includes the CAG region and has less than 36 CAG repeats in that region) into one or more glial cells and/or one or more neurons present within the brain of a mammal can be a particle-based method. Examples of methods that can be used to introduce donor nucleic acid into one or more glial cells and/or one or more neurons present within the brain of a mammal include, without limitation, electroporation, hydrodynamic delivery, transfection (e.g., lipofection), transduction (e.g., viral vector mediated transduction), lipid nanoparticles, lipoplexes, cell penetrating peptides, DNA nanoclew, gold nanoparticles, induced transduction by osmocytosis and propanebetaine (iTOP), microinjection, intravenous injection, intramuscular injection, and intranasal spray. In some cases, donor nucleic acid can be transduced into one or more glial cells and/or one or more neurons present within the brain of a mammal.

In some cases, gene editing (e.g., with engineered nucleases) can be used to modify one or more Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease). For example, gene editing can include a nuclease, a targeting sequence (e.g., a nucleic acid sequence that is complementary to at least a portion of one or both Htt genes), and, optionally, a donor nucleic acid (e.g., a nucleic acid including at least a fragment of a donor Htt gene having a CAG region with less than 36 CAG repeats and/or a modification that reduces or prevents expression of a huntingtin polypeptide having more than 11 consecutive glutamine residues). Nucleases useful for genome editing include, without limitation, Cas nucleases, zinc finger nucleases (ZFNs), transcription activator-like effector (TALE) nucleases (TALENs), and homing endonucleases (HE; also referred to as meganucleases). A targeting sequence can be used to direct a nuclease to particular target sequence within a genome (e.g., a target within one or both Htt genes present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease).

In some cases, a CRISPR/Cas system can be used (e.g., can be introduced into one or more glial cells) to modify the number of CAG repeats present in one or both Htt genes present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease).

CRISPR/Cas molecules are components of a prokaryotic adaptive immune system that is functionally analogous to eukaryotic RNA interference, using RNA base pairing to direct nucleic acid cleavage resulting in double stranded breaks (DSBs) about three to four nucleotides upstream of a protospacer adjacent motif (PAM) sequence (e.g., NGG). Directing nucleic acid DSBs with the CRISPR/Cas system requires two components: a Cas nuclease, and a guide RNA (gRNA) targeting sequence directing the Cas to cleave a target DNA sequence (Makarova et al., Nat Rev Microbiol, 9(6):467-477 (2011); and Jinek et al., Science, 337(6096):816-821 (2012)). The CRISPR/Cas system can be used in bacteria, yeast, humans, and zebrafish, as described elsewhere (see, e.g., Jiang et al., Nat Biotechnol, 31(3):233-239 (2013); Dicarlo et al., Nucleic Acids Res, doi:10.1093/nar/gkt135, 2013; Cong et al., Science, 339(6121):819-823 (2013); Mali et al., Science, 339(6121):823-826 (2013); Cho et al., Nat Biotechnol, 31(3):230-232 (2013); and Hwang et al., Nat Biotechnol, 31(3):227-229 (2013)).

In some cases, a CRISPR/Cas system used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can include any appropriate gRNA. In some cases, a gRNA can be complementary to at least a portion of a Htt gene present in one or more glial cells and/or one or more neurons present within the brain of a mammal.

In some cases, a CRISPR/Cas system used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can include any appropriate Cas nuclease. Examples of Cas nucleases include, without limitation, Cas1, Cas2, Cas3, Cas9, Cas10, and Cpf1. In some cases, a Cas component of a CRISPR/Cas system designed to modify the number of CAG repeats present in one or both Htt genes present in one or more glial cells and/or one or more neurons present within the brain of a mammal can be a Cas9 nuclease. For example, the Cas9 nuclease of a CRISPR/Cas9 system described herein can be a lentiCRISPRv2 (see, e.g., Shalem et al., 2014 Science 343:84-87; and Sanjana et al., 2014 Nature methods 11: 783-784).

In some cases, a TALEN system can be used (e.g., can be introduced into one or more glial cells) to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease). Transcription activator-like (TAL) effectors are found in plant pathogenic bacteria of the genus Xanthomonas. These proteins play important roles in disease, or trigger defense, by binding host DNA and activating effector-specific host genes (see, e.g., Gu et al., Nature 435:1122-1125, 2005; Yang et al., Proc Natl Acad Sci USA 103:10503-10508, 2006; Kay et al., Science 318:648-651, 2007; Sugio et al., Proc Natl Acad Sci USA 104:10720-10725, 2007; and Römer et al., Science 318:645-648, 2007). Specificity depends on an effector-variable number of imperfect, typically 34 amino acid repeats (Schornack et al., J Plant Physiol 163:256-272, 2006; and WO 2011/072246). Polymorphisms are present primarily at repeat positions 12 and 13, which are referred to as the repeat variable-diresidue (RVD). The RVDs of TAL effectors correspond to the nucleotides in their target sites in a direct, linear fashion, one RVD to one nucleotide, with some degeneracy and no apparent context dependence. This mechanism for protein-DNA recognition enables target site selection and engineering of new TALENs with binding specificity for the selected sites. For example, an engineered TAL effector DNA binding domain targeting sequence can be fused to a nuclease to create a TALEN that can create nucleic acid DSBs at or near the sequence targeted by the TAL effector DNA binding domain. Directing nucleic acid DSBs with the TALEN system requires two components: a nuclease, and TAL effector DNA-binding domain directing the nuclease to a target DNA sequence (see, e.g., Schornack et al., J. Plant Physiol. 163:256, 2006).

A TALEN system used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can include any appropriate nuclease. In some cases, a nuclease can be a non-specific nuclease. In some cases, a nuclease can function as a dimer. For example, when a nuclease that functions as a dimer is used, a highly site-specific restriction enzyme can be created. For example, each nuclease monomer can be fused to a TAL effector sequence that recognizes a different DNA target sequence, and only when the two recognition sites are in close proximity do the inactive monomers come together to create a functional enzyme. Examples of nucleases that can used in a TALEN system described herein include, without limitation, FokI, HhaI, HindIII, NotI, BbvCI, EcoRI, BglI, and AlwI. For example, a nuclease of a TALEN system can include a FokI nuclease (see, e.g., Kim et al. (1996) Proc. Natl. Acad. Sci. USA 93:1156-1160).

A TALEN system used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can include any appropriate TAL effector DNA-binding domain. In some cases, TAL effector DNA-binding domain can be complementary to a Htt gene present in a mammal.

When a gene editing system (e.g., a CRISPR/Cas system or a TALEN system) is used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease), the system can optionally include donor nucleic acid (e.g., donor nucleic acid including a fragment of an Htt gene that includes the CAG region and has less than 36 CAG repeats in that region). For example, in the presence of the donor nucleic acid, a gene editing system can modify one or both Htt genes present in one or more glial cells and/or one or more neurons present within the brain of a mammal, such that the modified Htt gene(s) can encode a functional HTT polypeptide within the brain of the mammal. Components of a gene editing system (e.g., CRISPR/Cas system or a TALEN system) used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can be introduced into the one or more glial cells and/or the one or more neurons present in any appropriate format. In some cases, a component of a CRISPR/Cas system can be introduced into one or more glial cells and/or one or more neurons as nucleic acid encoding a gRNA and/or nucleic acid encoding a Cas nuclease. For example, nucleic acid encoding at least one gRNA (e.g., a gRNA sequence specific to a Htt gene present in a mammal) and nucleic acid encoding at least one Cas nuclease (e.g., a Cas9 nuclease) can be introduced into one or more glial cells and/or one or more neurons present within the brain of a mammal. In some cases, a component of a CRISPR/Cas system can be introduced into one or more glial cells and/or one or more neurons as a gRNA and/or as a Cas nuclease. For example, at least one gRNA (e.g., a gRNA sequence specific to a Htt gene present in a mammal) and at least one Cas nuclease (e.g., a Cas9 nuclease) can be introduced into one or more glial cells. In some cases, TALENs can be introduced into one or more glial cells and/or one or more neurons as nucleic acid encoding a TALEN. In some cases, TALENs can be introduced into one or more glial cells as TALEN polypeptide.

In some cases, when components of a gene editing system (e.g., a CRISPR/Cas system or a TALEN system) are introduced into one or more glial cells (e.g., astrocytes) and/or one or more neurons (e.g., astrocyte-converted neurons and/or non-converted neurons) present within the brain (e.g., striatum) of a mammal (e.g., a human having Huntington's disease) as nucleic acid encoding the components (e.g., nucleic acid encoding a gRNA and nucleic acid encoding a Cas nuclease, or nucleic acid encoding a TALEN), the nucleic acid can be any appropriate form. For example, nucleic acid can be a construct (e.g., an expression construct). When a gene editing system is a CRISPR/Cas system, nucleic acid encoding at least one gRNA and nucleic acid encoding at least one Cas nuclease can be on separate nucleic acid constructs or on the same nucleic acid construct. In some cases, nucleic acid encoding at least one gRNA and nucleic acid encoding at least one Cas nuclease can be on a single nucleic acid construct. A nucleic acid construct can be any appropriate type of nucleic acid construct. Examples of nucleic acid constructs that can be used to express at least one component of a gene editing system include, without limitation, expression plasmids and viral vectors (e.g., lentiviral vectors). When a gene editing system is a CRISPR/Cas system, and in cases where nucleic acid encoding at least one gRNA and nucleic acid encoding at least one Cas nuclease are on separate nucleic acid constructs, the nucleic acid constructs can be the same type of construct or different types of constructs.

In some cases, one or more components of a gene editing system (e.g., a CRISPR/Cas system or a TALEN system) can be introduced directly into one or more glial cells (e.g., astrocytes) and/or one or more neurons (e.g., astrocyte-converted neurons and/or non-converted neurons) present within the brain (e.g., striatum) of a mammal (e.g., a human having Huntington's disease) as a polypeptide. When a gene editing system is a CRISPR/Cas system, a gRNA and a Cas nuclease can be introduced into the one or more glial cells and/or one or more neurons separately or together. In cases where a gRNA and a Cas nuclease are introduced into the one or more glial cells and/or the one or more neurons together, the gRNA and the Cas nuclease can be in a complex. When a gRNA and a Cas nuclease are in a complex, the gRNA and the Cas nuclease can be covalently or non-covalently attached.

Components of a gene editing system (e.g., a CRISPR/Cas system or a TALEN system) used to modify one or both Htt alleles present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease) can be introduced into one or more glial cells and/or one or more neurons using any appropriate method. A method of introducing components of a gene editing system into one or more glial cells and/or one or more neurons present within the brain of a mammal can be a physical method. A method of introducing components of a gene editing system into one or more glial cells and/or one or more neurons present within the brain of a mammal can be a chemical method. A method of introducing components of a gene editing system into one or more glial cells and/or one or more neurons present within the brain of a mammal can be a particle-based method. Examples of methods that can be used to introduce components of a gene editing system into one or more glial cells and/or one or more neurons present within the brain of a mammal include, without limitation, electroporation, hydrodynamic delivery, transfection (e.g., lipofection), transduction (e.g., viral vector mediated transduction), lipid nanoparticles, lipoplexes, cell penetrating peptides, DNA nanoclew, gold nanoparticles, induced transduction by osmocytosis and propanebetaine (iTOP), and microinjection. In some cases, when components of a gene editing system are introduced into one or more glial cells and/or one or more neurons as nucleic acid encoding the components, the nucleic acid encoding the components can be transduced into the one or more glial cells and/or one or more neurons.

In some cases, a mammal (e.g., a human) having Huntington's disease can be treated using a method that converts glial cells into neurons and corrects the CAG repeats together as a single treatment, or at different times as two or more treatments.

In some cases, a mammal (e.g., a human) having Huntington's disease can be treated using a method that converts glial cells into neurons and deactivates an Htt allele that expresses a huntingtin polypeptide having more than 11 consecutive glutamine residues together as a single treatment, or at different times as two or more treatments.

In some cases, a treatment as provided herein is administered to a mammal (e.g., a human) having Huntington's disease at least once daily or at least once weekly for at least two consecutive days or weeks. In some cases, a treatment as provided herein is administered to a mammal (e.g., a human) having Huntington's disease at least 3, 4, 5, 6, 7, 8, 9, 10, 11, 12, 13, 14, or 15 consecutive days or weeks. In some cases, a treatment as provided herein is administered to a mammal (e.g., a human) having Huntington's disease at least once daily or at least once weekly for at least 1, 2, 3, 4, 5, 6, 7, 8, 9, 10, 11, or 12 consecutive weeks. In some cases, a treatment as provided herein is administered to a mammal (e.g., a human) having Huntington's disease at least once daily or at least once weekly for at most 4, 5, 6, 7, 8, 9, 10, 11, 12, 13, 14, 15, 16, 17, 18, 19, or 20 consecutive days or weeks. In some cases, a treatment as provided herein is administered to a mammal (e.g., a human) having Huntington's disease at least once weekly for at most 1, 2, 3, 4, 5, 6, 7, 8, 9, 10, 11, or 12 consecutive weeks or months. In some cases, a treatment as provided herein is administered to a mammal (e.g., a human) having Huntington's disease at least 1, 2, 3, 4, 5, 6, 7, 8, 9, 10, 11, or 12 consecutive months or years, chronically for a subject's entire life span, or an indefinite period of time.

In some cases, the methods and materials described herein can be used to slow, delay, or reverse the progression of Huntington's disease. For example, the methods and materials described herein delay the onset of one or more symptoms of Huntington's disease and/or to reduce or eliminate one or more symptoms of Huntington's disease. In some cases, the regeneration of new functional neurons and editing of an Htt allele in combination has a synergistic effect on delaying the onset of one or more symptoms of Huntington's disease and/or reducing or eliminating one or more symptoms of Huntington's disease.

Examples of tests evaluating the slowing, delaying, or reversal of Huntington's disease progression include, but not limited to, the unified Huntington's disease rating scale (UHDRS) score, UHDRS Total Functional Capacity (TFC), UHDRS Functional Assessment, UHDRS Gait score, UHDRS Total Motor Score (TMS), Hamilton depression scale (HAM-D), Columbia-suicide severity rating scale (C-SSRS), Montreal cognitive assessment (MoCA), MRI, fMRI, and PET scan.

In some cases, a symptom can be slowed or delayed by from about 10 percent to about 99 percent or more. In some cases, a symptom can be slowed or delayed from about 10 percent to about 100 percent, from about 10 percent to about 15 percent, from about 10 percent to about 20 percent, from about 10 percent to about 25 percent, from about 15 percent to about 20 percent, from about 15 percent to about 25 percent, from about 15 percent to about 30 percent, from about 20 percent to about 25 percent, from about 20 percent to about 30 percent, from about 20 percent to about 35 percent, from about 25 percent to about 30 percent, from about 25 percent to about 35 percent, from about 25 percent to about 40 percent, from about 30 percent to about 35 percent, from about 30 percent to about 40 percent, from about 35 percent to about 45 percent, from about 35 percent to about 50 percent, from about 40 percent to about 45 percent, from about 40 percent to about 50 percent, from about 40 percent to about 55 percent, from about 45 percent to about 50 percent, from about 45 percent to about 55 percent, from about 45 percent to about 60 percent, from about 50 percent to about 55 percent, from about 50 percent to about 60 percent, from about 50 percent to about 65 percent, from about 55 percent to about 60 percent, from about 55 percent to about 65 percent, from about 55 percent to about 70 percent, from about 60 percent to about 65 percent, from about 60 percent to about 70 percent, from about 60 percent to about 75 percent, from about 65 percent to about 70 percent, from about 65 percent to about 75 percent, from about 65 percent to about 80 percent, from about 70 percent to about 75 percent, from about 70 percent to about 80 percent, from about 70 percent to about 85 percent, from about 75 percent to about 80 percent, from about 75 percent to about 85 percent, from about 75 percent to about 90 percent, from about 80 percent to about 85 percent, from about 80 percent to about 90 percent, from about 80 percent to about 95 percent, from about 85 percent to about 90 percent, from about 85 percent to about 95 percent, from about 85 percent to about 100 percent, from about 90 percent to about 95 percent, from about 90 percent to about 100 percent, or from about 95 percent to about 100 percent.

In some cases, symptoms can be assessed on the day of treatment, 1 day post treatment, 3 months post treatment, 6 months post treatment, 1 year post treatment and every year thereafter post treatment.

In some cases, symptoms can be assessed between 1 day post treatment and 7 days post treatment. In some cases, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In some cases, symptoms can be assessed between 1 week post treatment and 4 weeks post treatment. In some cases, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In some cases, symptoms can be assessed between 1 month post treatment and 12 months post treatment. In some cases, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In some cases, symptoms can be assessed between 1 year post treatment and about 20 years post treatment. In some cases symptoms can be assessed between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

In some cases, a symptom of Huntington's disease can be a movement symptom (e.g., an impairment in one or more motor functions). For example, a movement symptom can be an impairment of an involuntary movement or an impairment of a voluntary movement. In some cases, a symptom of Huntington's disease can be a cognitive symptom. In some cases, a symptom of Huntington's disease can be a psychiatric symptom. Examples of symptoms of Huntington's disease that can be reduced or eliminated using the methods and materials described herein include, without limitation, changes (e.g., reduction or loss of) fine motor skills, tremors, seizures, chorea, dystonia, dyskinesia, slow or abnormal eye movements, impaired gait, impaired posture, impaired balance, difficulty with speech, difficulty with swallowing, difficulty organizing, difficulty prioritizing, difficulty focusing on tasks, lack of flexibility, lack of impulse control, outbursts, lack of awareness of one's own behaviors and/or abilities, slowness in processing thoughts, difficulty in learning new information, depression, irritability, sadness or apathy, social withdrawal, insomnia, fatigue, lack of energy, obsessive-compulsive disorder, mania, bipolar disorder, and weight loss.

In some cases, a symptom can be reduced by from about 10 percent to about 99 percent or more. In some cases, a symptom can be reduced from about 10 percent to about 100 percent, from about 10 percent to about 15 percent, from about 10 percent to about 20 percent, from about 10 percent to about 25 percent, from about 15 percent to about 20 percent, from about 15 percent to about 25 percent, from about 15 percent to about 30 percent, from about 20 percent to about 25 percent, from about 20 percent to about 30 percent, from about 20 percent to about 35 percent, from about 25 percent to about 30 percent, from about 25 percent to about 35 percent, from about 25 percent to about 40 percent, from about 30 percent to about 35 percent, from about 30 percent to about 40 percent, from about 35 percent to about 45 percent, from about 35 percent to about 50 percent, from about 40 percent to about 45 percent, from about 40 percent to about 50 percent, from about 40 percent to about 55 percent, from about 45 percent to about 50 percent, from about 45 percent to about 55 percent, from about 45 percent to about 60 percent, from about 50 percent to about 55 percent, from about 50 percent to about 60 percent, from about 50 percent to about 65 percent, from about 55 percent to about 60 percent, from about 55 percent to about 65 percent, from about 55 percent to about 70 percent, from about 60 percent to about 65 percent, from about 60 percent to about 70 percent, from about 60 percent to about 75 percent, from about 65 percent to about 70 percent, from about 65 percent to about 75 percent, from about 65 percent to about 80 percent, from about 70 percent to about 75 percent, from about 70 percent to about 80 percent, from about 70 percent to about 85 percent, from about 75 percent to about 80 percent, from about 75 percent to about 85 percent, from about 75 percent to about 90 percent, from about 80 percent to about 85 percent, from about 80 percent to about 90 percent, from about 80 percent to about 95 percent, from about 85 percent to about 90 percent, from about 85 percent to about 95 percent, from about 85 percent to about 100 percent, from about 90 percent to about 95 percent, from about 90 percent to about 100 percent, or from about 95 percent to about 100 percent. For example, the methods and materials described herein can be used to improve one or more motor function deficits in a mammal (e.g., a human) with Huntington's disease. For example, methods and materials described herein can be used to rescue (e.g., partially rescue or completely rescue) one or more motor function deficits in a mammal (e.g., a human) with Huntington's disease. In some cases, the regeneration of new functional neurons and editing of an Htt allele in combination has a synergistic effect on improving one or more motor function deficits in a mammal (e.g., a human) with Huntington's disease.

Any appropriate method can be used to evaluate motor function deficits in a mammal with Huntington's disease. For example, body weight, clasping behavior, grip strength gait, hand and leg movement, and/or specific limb coordination can be used to evaluate motor function deficits in a mammal with Huntington's disease.

In some cases, motor function deficits can be evaluated on the day of treatment, 1 day post treatment, 3 months post treatment, 6 months post treatment, 1 year post treatment and every year thereafter post treatment.

In some cases, motor function deficits can be evaluated between 1 day post treatment and 7 days post treatment. In some cases, motor function deficits can be evaluated between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In some cases, motor function deficits can be evaluated between 1 week post treatment and 4 weeks post treatment. In some cases, motor function deficits can be evaluated between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In some cases, motor function deficits can be evaluated between 1 month post treatment and 12 months post treatment. In some cases, motor function deficits can be evaluated between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In some cases, motor function deficits can be evaluated between 1 year post treatment and about 20 years post treatment. In some cases, motor function deficits can be evaluated between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment. In some cases, the methods and materials described herein can be used to extend the life expectancy of a mammal (e.g., a human) with Huntington's disease. For example, the life expectancy of a mammal with Huntington's disease can be extended by from about 2 years to about 20 years or longer (e.g., as compared to the life expectancy of a mammal with Huntington's disease that is not treated as described herein). In some cases, the regeneration of new functional neurons and editing of an Htt allele in combination has a synergistic effect on extending the life expectancy of a mammal (e.g., a human) with Huntington's disease. In some cases, the life expectancy of a mammal with Huntington's can be extended from about 2 years to about 5 years, from about 2 years to about 10 years, from about 2 years to about 15 years, from about 5 years to 10 years, from about 5 years to about 15 years, from about 5 years to about 20 years, from about 10 years to about 15 years, from about 10 years to about 20 years, or from about 15 years to about 20 years. For example, the life expectancy of a mammal with Huntington's disease can be extended by from about 10 percent to about 60 percent or more (e.g., as compared to the life expectancy of a mammal with Huntington's disease that is not treated as described herein). In some cases, the life expectancy can be reduced by 10 percent to about 15 percent, from about 10 percent to about 20 percent, from about 10 percent to about 25 percent, from about 15 percent to about 20 percent, from about 15 percent to about 25 percent, from about 15 percent to about 30 percent, from about 20 percent to about 25 percent, from about 20 percent to about 30 percent, from about 20 percent to about 35 percent, from about 25 percent to about 30 percent, from about 25 percent to about 35 percent, from about 25 percent to about 40 percent, from about 30 percent to about 35 percent, from about 30 percent to about 40 percent, from about 35 percent to about 45 percent, from about 35 percent to about 50 percent, from about 40 percent to about 45 percent, from about 40 percent to about 50 percent, from about 40 percent to about 55 percent, from about 45 percent to about 50 percent, from about 45 percent to about 55 percent, from about 45 percent to about 60 percent, from about 50 percent to about 55 percent, from about 50 percent to about 60 percent, or from about 55 percent to about 60 percent. In some cases, the methods and materials described herein can be used to reduce or eliminate atrophy present within the brain (e.g., striatum) of a mammal (e.g., a human having Huntington's disease). For example, the methods and materials described herein can be effective to reduce the amount of atrophy within the brain of a mammal with Huntington's disease by, for example, 10, 20, 30, 40, 50, 60, 70, 80, 90, 95, or more percent (e.g., as compared to the amount of atrophy in native neurons in a mammal with Huntington's disease such as neurons in a mammal that has not been treated as described herein and/or neurons in a mammal prior to being treated as described herein). The methods and materials described herein can be effective to reduce the amount of atrophy within the brain of a mammal with Huntington's disease from 10 percent to 100 percent, such as from 10 percent to 15 percent, from 10 percent to 20 percent, from 10 percent to 25 percent, from 15 percent to 20 percent, from 15 percent to 25 percent, from 15 percent to 30 percent, from 20 percent to 25 percent, from 20 percent to 30 percent, from 20 percent to 35 percent, from 25 percent to 30 percent, from 25 percent to 35 percent, from 25 percent to 40 percent, from 30 percent to 35 percent, from 30 percent to 40 percent, from 35 percent to 45 percent, from 35 percent to 50 percent, from 40 percent to 45 percent, from 40 percent to 50 percent, from 40 percent to 55 percent, from 45 percent to 50 percent, from 45 percent to 55 percent, from 45 percent to 60 percent, from 50 percent to 55 percent, from 50 percent to 60 percent, from 50 percent to 65 percent, from 55 percent to 60 percent, from 55 percent to 65 percent, from 55 percent to 70 percent, from 60 percent to 65 percent, from 60 percent to 70 percent, from 60 percent to 75 percent, from 65 percent to 70 percent, from 65 percent to 75 percent, from 65 percent to 80 percent, from 70 percent to 75 percent, from 70 percent to 80 percent, from 70 percent to 85 percent, from 75 percent to 80 percent, from 75 percent to 85 percent, from 75 percent to 90 percent, from 80 percent to 85 percent, from 80 percent to 90 percent, from 80 percent to 95 percent, from 85 percent to 90 percent, from 85 percent to 95 percent, from 85 percent to 100 percent, from 90 percent to 95 percent, from 90 percent to 100 percent, or from 95 percent to 100 percent. Any appropriate method can be used to evaluate the presence, absence, or amount of atrophy within the brain of a mammal having Huntington's disease. For example, Nissle staining, MRI, fMRI, and/or PET scanning can be used to evaluate the presence, absence, or amount of atrophy within the brain of a mammal.

In some cases, the presence, absence, or amount of atrophy can be evaluated on the day of treatment, 1 day post treatment, 3 months post treatment, 6 months post treatment, 1 year post treatment and every year thereafter post treatment.

In some cases, the presence, absence, or amount of atrophy can be evaluated between 1 day post treatment and 7 days post treatment. In some cases, the presence, absence, or amount of atrophy can be evaluated between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In some cases, the presence, absence, or amount of atrophy can be evaluated between 1 week post treatment and 4 weeks post treatment. In some case, the presence, absence, or amount of atrophy can be evaluated between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In some cases, the presence, absence, or amount of atrophy can be evaluated between 1 month post treatment and 12 months post treatment. In some cases, the presence, absence, or amount of atrophy between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In some cases, the presence, absence, or amount of atrophy can be evaluated between 1 year post treatment and about 20 years post treatment. In some cases, the presence, absence, or amount of atrophy can be evaluated between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment. In some cases, the methods and materials described herein can be used to reduce or eliminate the amount of HTT polypeptide inclusions (e.g., nuclear HTT polypeptide inclusions) present in one or more glial cells (e.g., astrocytes) and/or one or more neurons (e.g., astrocyte-converted neurons and/or non-converted neurons) present within the brain (e.g., striatum) of a mammal (e.g., a human having Huntington's disease). A HTT polypeptide inclusion can be in any appropriate location within a cell. For example, a HTT polypeptide inclusion can be a nuclear HTT polypeptide inclusion. In some cases, the methods and materials described herein can be effective to reduce the amount of HTT polypeptide inclusions present in one or more glial cells and/or one or more neurons present within the brain of a mammal with Huntington's disease by, for example, 10, 20, 30, 40, 50, 60, 70, 80, 90, 95, or more percent (e.g., as compared to the amount of HTT polypeptide inclusions in native neurons in a mammal with Huntington's disease such as neurons in a mammal that has not been treated as described herein and/or neurons in a mammal prior to being treated as described herein). In some cases, the methods and materials described herein can be effective to reduce the amount of HTT polypeptide inclusions present in one or more glial cells and/or one or more neurons present within the brain of a mammal from 10 percent to 100 percent, such as from 10 percent to 15 percent, from 10 percent to 20 percent, from 10 percent to 25 percent, from 15 percent to 20 percent, from 15 percent to 25 percent, from 15 percent to 30 percent, from 20 percent to 25 percent, from 20 percent to 30 percent, from 20 percent to 35 percent, from 25 percent to 30 percent, from 25 percent to 35 percent, from 25 percent to 40 percent, from 30 percent to 35 percent, from 30 percent to 40 percent, from 35 percent to 45 percent, from 35 percent to 50 percent, from 40 percent to 45 percent, from 40 percent to 50 percent, from 40 percent to 55 percent, from 45 percent to 50 percent, from 45 percent to 55 percent, from 45 percent to 60 percent, from 50 percent to 55 percent, from 50 percent to 60 percent, from 50 percent to 65 percent, from 55 percent to 60 percent, from 55 percent to 65 percent, from 55 percent to 70 percent, from 60 percent to 65 percent, from 60 percent to 70 percent, from 60 percent to 75 percent, from 65 percent to 70 percent, from 65 percent to 75 percent, from 65 percent to 80 percent, from 70 percent to 75 percent, from 70 percent to 80 percent, from 70 percent to 85 percent, from 75 percent to 80 percent, from 75 percent to 85 percent, from 75 percent to 90 percent, from 80 percent to 85 percent, from 80 percent to 90 percent, from 80 percent to 95 percent, from 85 percent to 90 percent, from 85 percent to 95 percent, from 85 percent to 100 percent, from 90 percent to 95 percent, from 90 percent to 100 percent, or from 95 percent to 100 percent.

Any appropriate method can be used to evaluate the presence, absence, or amount of HTT polypeptide inclusions in a mammal with Huntington's disease. For example, immunohistochemistry can be used to evaluate the presence, absence, or amount of HTT polypeptide inclusions present in one or more glial cells and/or one or more neurons present within the brain of a mammal with Huntington's disease. In some cases, the presence, absence, or amount of HTT polypeptide inclusions can be evaluated the day of treatment, 1 day post treatment, 3 months post treatment, 6 months post treatment, 1 year post treatment and every year thereafter post treatment.

In some cases, the presence, absence, or amount of HTT polypeptide inclusions can be evaluated between 1 day post treatment and 7 days post treatment. In some cases, the presence, absence, or amount of HTT polypeptide inclusions can be evaluated between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In some cases, the presence, absence, or amount of HTT polypeptide inclusions can be evaluated between 1 week post treatment and 4 weeks post treatment. In some cases, the presence, absence, or amount of HTT polypeptide inclusions can be evaluated between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In some cases, the presence, absence, or amount of HTT polypeptide inclusions can be evaluated between 1 month post treatment and 12 months post treatment. In some cases, the presence, absence, or amount of HTT polypeptide inclusions between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In some cases, the presence, absence, or amount of HTT polypeptide inclusions can be evaluated between 1 year post treatment and about 20 years post treatment. In some cases, the presence, absence, or amount of HTT polypeptide inclusions can be evaluated between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

The invention will be further described in the following examples, which do not limit the scope of the invention described in the claims.

### EXAMPLES

### Example 1—a Gene Therapy Approach to Directly Convert Striatal Astrocytes into GABAergic Neurons in a Mouse Model of Huntington's Disease

### Targeting Striatal Astrocytes for In Vivo Neuronal Conversion

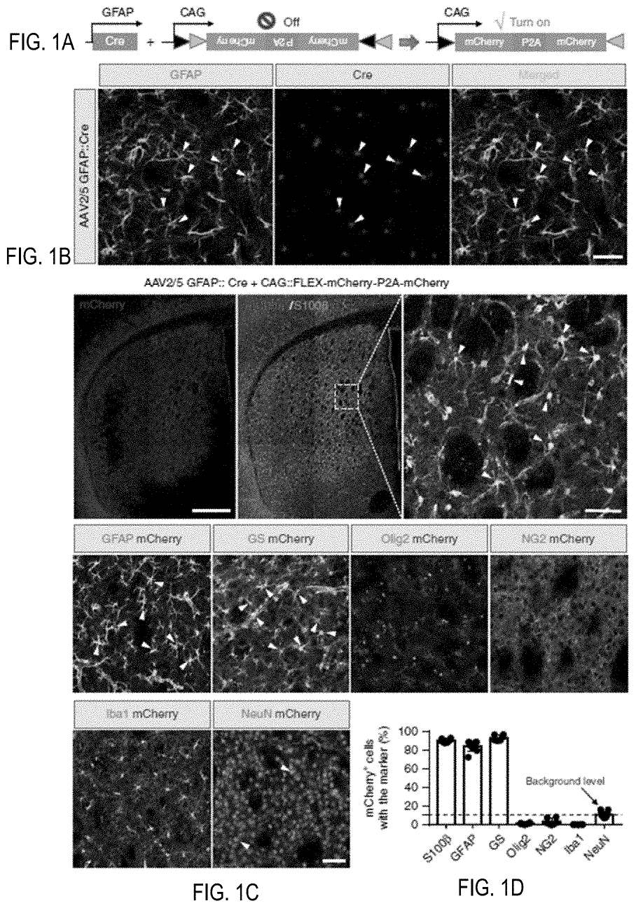

Astrocytes are abundant cells that make up approximately 30% of the cells in the mammalian CNS and essentially surround every single neuron in the brain, making them an ideal internal source for cell conversion. Ectopic expression of a single neural transcription factor, NeuroD1, in cortical astrocytes can convert them into functional neurons, mainly glutamatergic neurons (Guo et al., Cell Stem Cell 14:188-202 (2014)). However, the total number of in vivo converted neurons by retroviruses is limited, because retroviruses can only express target genes in dividing cells. To overcome this disadvantage of retroviruses, recombinant adeno-associated virus (serotype 2/5, rAAV2/5) for in vivo reprogramming were designed. Among different serotypes of rAAV, rAAV2/5 was used for its ability to infect astrocytes preferentially in the mouse brain (Ortinski et al., Nat. Neurosci. 13:584-591 (2010)). To track the astrocyte-converted neurons in the mouse brain, a Cre-FLEx (flip-excision) system was developed that includes a vector expressing Cre recombinase under the control of the GFAP promoter (GFAP::Cre) to target astrocytes, and FLEx vectors with an inverted coding sequence of mCherry-P2A-mCherry or NeuroD1-P2A-mCherry or Dlx2-P2A-mCherry (FIG. 1a). The two inserted genes are separated by P2A self-cleavage site and driven by the strong universal synthetic promoter CAG. It was tested whether Dlx2 in combination with NeuroD1 can convert striatal astrocytes into GABAergic neurons, and whether NeuroD1 alone might generate more glutamatergic neurons (Guo et al., Cell Stem Cell 14:188-202 (2014)).

To test whether Cre-recombinase is specifically over-expressed in the astrocytes, AAV2/5 GFAP::Cre was injected into the normal mouse striatum (2-5 months), a brain region enriched with GABAergic neurons, which shows early degeneration in HD brains. Almost all of the Cre-expressing cells were GFAP-positive cells, a typical marker for astrocytes (99.2±0.6%, n=6 mice, 7-21 days post viral injection; FIG. 1b). In order to further investigate the specificity of the Cre-FLEx system, AAV2/5 GFAP::Cre was injected together with AAV2/5-CAG::FLEx-mCherry-P2A-mCherry into the normal mouse striatum. The mice were sacrificed at 21-30 days post-injection (dpi) for immunohistological studies. Of the mCherry-positive cells, the majority of them expressed astrocyte-specific markers including S100β (90.0±0.9%), GFAP (86.6±1.9%), and glutamine synthetase (GS, 92.9±1.3%), with very few expressing other glial markers such as Olig2 (1.1±0.3%), NG2 (3.2±1.5%) and Iba1 (not detected, n≥6 mice for each group; FIG. 1c, d). A few mCherry-expressing cells were NeuN-positive (10.5±0.7%, n=11 mice; FIG. 1c, d), indicating that a very small number of striatal neurons was targeted by the AAV2/5 Cre-FLEx system.

NeuroD1 and Dlx2 Reprogram Striatal Astrocytes into GABAergic Neurons

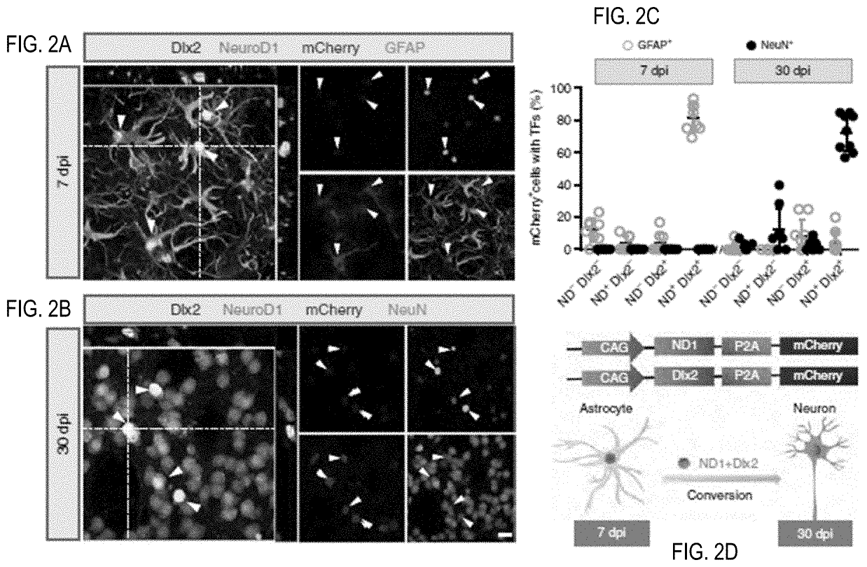

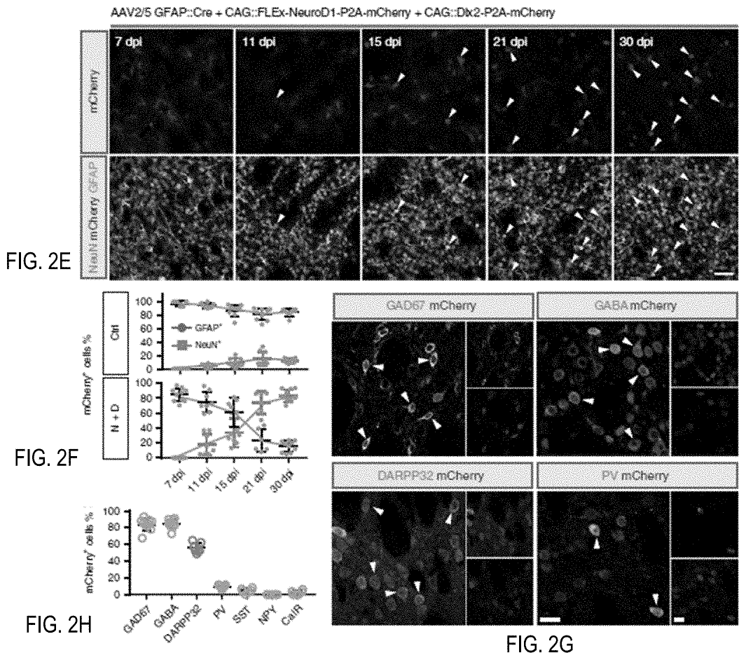

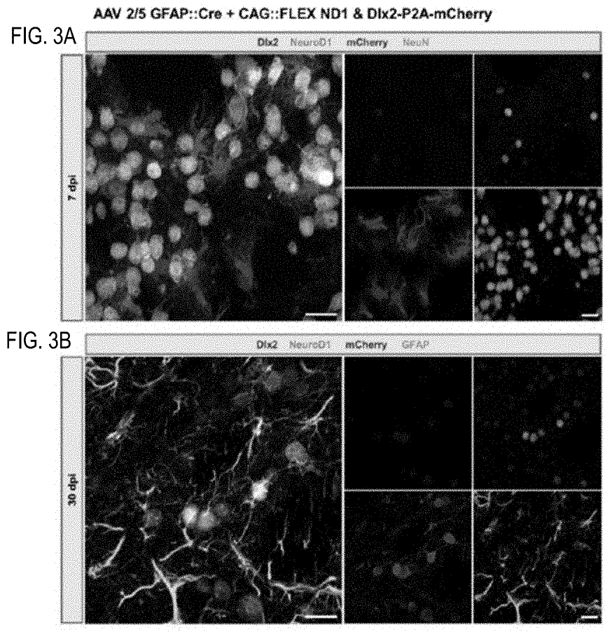

It was next tested whether a AAV Cre-FLEx system could drive the conversion of astrocytes into neurons in the striatum by injecting AAV2/5 GFAP::Cre together with AAV2/5-CAG::Dlx2-P2A-mCherry and CAG::NeuroD1-P2A-mCherry into adult wild type (WT) mice (age 2-5 months). At 7 dpi, it was found that all the viral infected cells (mCherry positive) in the striatum were GFAP+ astrocytes, among which 81.5% of the mCherry positive cells also co-expressed both NeuroD1 (ND1) and Dlx2, while only 12.1% of the mCherry positive cells showed neither ND1 nor Dlx2 expression (FIG. 2a, quantified in FIG. 2c). A small percentage (<5%) of the mCherry positive cells (mainly glial cells) only expressed one of the transcriptional factors (either ND1 positive or Dlx2 positive), but neither of the TFs were detected in NeuN positive neurons at 7 dpi (FIG. 2a and FIG. 3a). In contrast, by 30 dpi, it was found that most of the ND1 and Dlx2 signals were co-expressed in NeuN positive neurons (72.7%; FIG. 2b, quantified in FIG. 2c, black dots; FIG. 3b), with only a small number in astrocytes (4.1%, FIG. 2c, gray dots). These results suggest that coexpression of NeuroD1 and Dlx2 can convert striatal astrocytes into neurons (FIG. 2d).

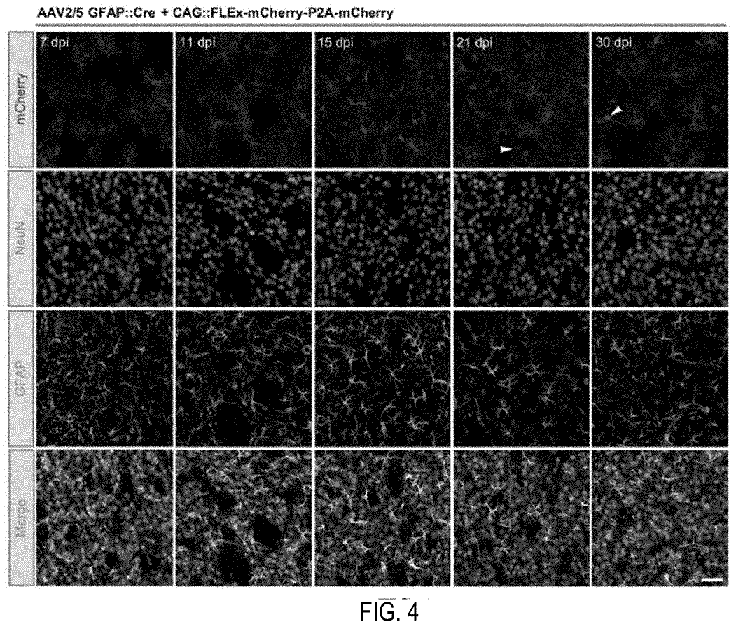

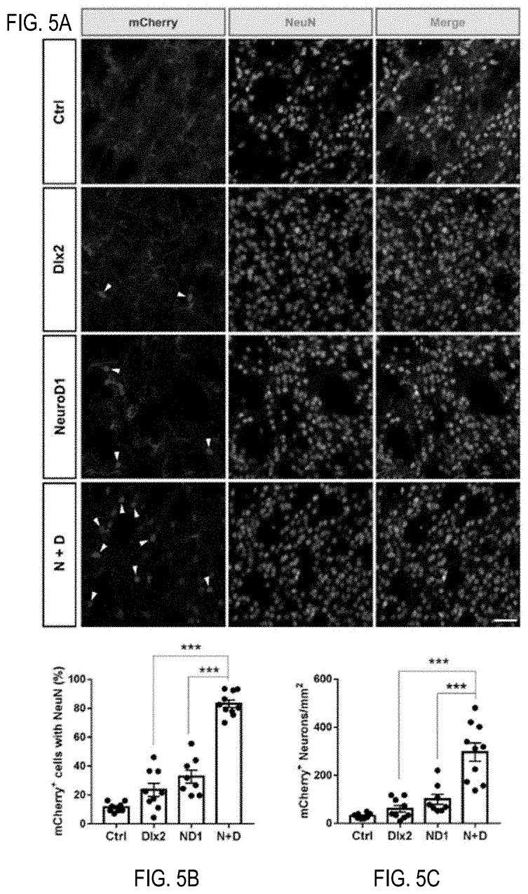

To further investigate the time course of the astrocyte-to-neuron conversion process in the striatum, three more time points of 11 dpi, 15 dpi, and 21 dpi were analyzed in addition to 7 dpi and 30 dpi (FIG. 2e). It was found that a small percentage (17.8%) of mCherry positive cells showed NeuN positive signal after co-expressing NeuroD1+Dlx2 (N+D) at 11 dpi, and such neuronal conversion percentage continuously increased to 33.6% at 15 dpi and 74.1% at 21 dpi (FIG. 2e, f). Parallel to this trend, more and more mCherry positive cells colocalized with NeuN, while less and less mCherry positive cells colocalized with GFAP from 7 dpi (83.5% GFAP+) to 30 dpi (14.2% GFAP+) (FIG. 2e, f). In the control group infected by AAV2/5 mCherry alone, most of the mCherry positive cells were GFAP+ astrocytes, with very few of the mCherry positive cells co-labeled with NeuN signal across the time points (FIG. 2f, FIG. 4). Because NeuroD1 or Dlx2 alone can convert astrocytes into neurons, their individual effects were further compared by injecting the mCherry control, NeuroD1, Dlx2, and NeuroD1+Dlx2 into WT mouse striatum. It was found that expressing either NeuroD1 or Dlx2 alone in striatal astrocytes also resulted in a number of the mCherry positive cells co-labeled with NeuN, but the conversion efficiency and the number of converted neurons were much lower than the NeuroD1+Dlx2 group (FIG. 5a-c). These results suggest that NeuroD1 and Dlx2 together have synergic effects in converting striatal astrocytes into neurons.

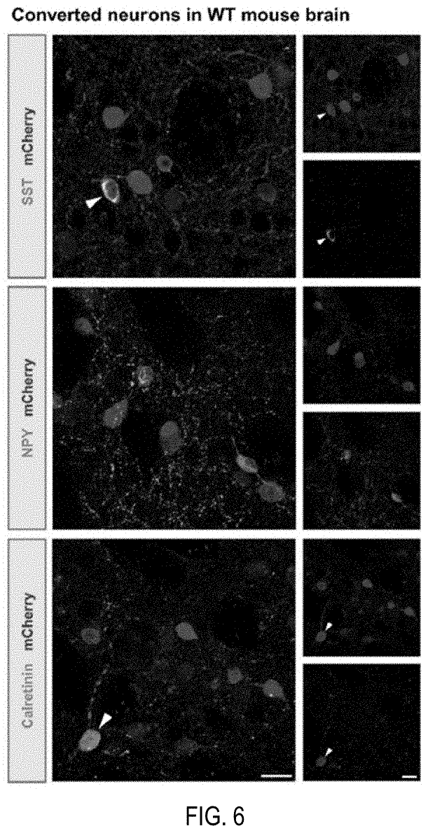

To identify the neuronal subtypes after NeuroD1+Dlx2 induced astrocyte-to-neuron conversion in the striatum, a series of immunostaining experiments was performed with a variety of GABAergic markers including GAD67 and GABA for GABAergic neurons; DARPP32 for MSNs; and paravalbumin (PV), somatostatin (SST), neuronal peptide Y, and calretinin (CalR) for striatal interneurons. It was found that most of the mCherry positive cells (30 dpi) were GAD67 positive (83.9%, n=10 mice) or GABA positive (85.0%, n=10 mice) GABAergic neurons (FIG. 2g, h). Furthermore, the majority of the converted neurons was DARPP32 positive (55.7%, n=7 mice; FIG. 2g, h), and a small percentage of the converted neurons was PV+ interneurons (9.6%; FIG. 2g, h), with even fewer other subtypes of interneurons (<5%; FIG. 2h, FIG. 6). To conclude, Dlx2 together with NeuroD1 can efficiently convert striatal astrocytes into DARPP32 positive GABAergic neurons.

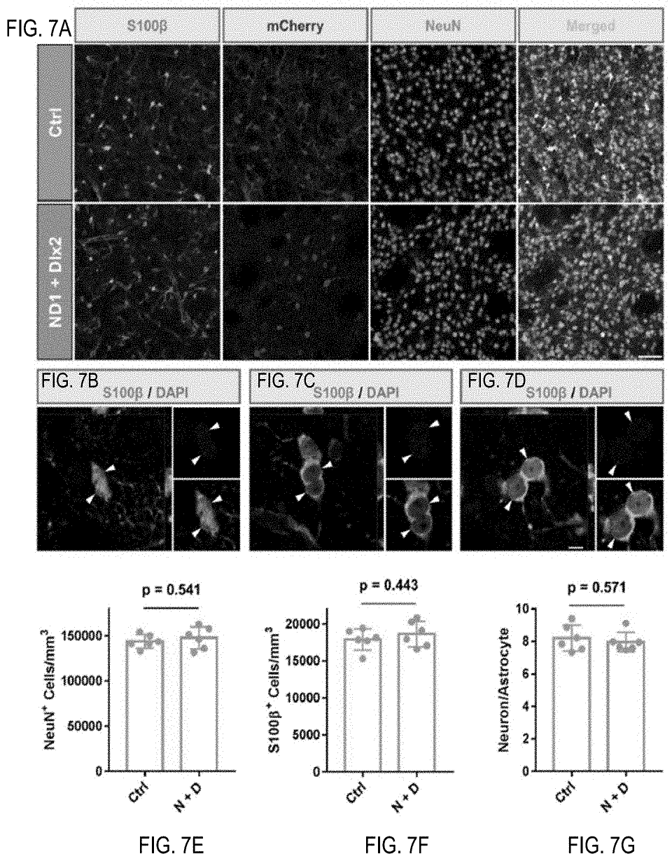

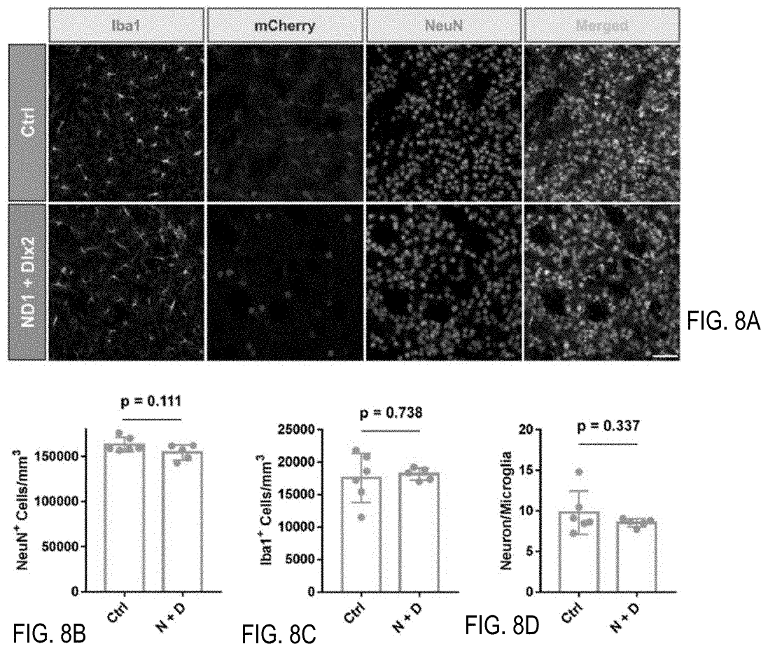

To examine whether the neuron to astrocyte ratio be altered after converting striatal astrocytes into neurons, the neuron/astrocyte ratio (FIG. 7) and neuron/microglia ratio (FIG. 8) were analyzed in the striatum at 30 days post AAV injection. According to the NeuN and S100β immunostaining, the overall neuronal and astrocytic density as well as the neuron/astrocyte ratio was not significantly changed after astrocyte-to-neuron conversion (FIG. 7). This might be due to the fact that astrocytes are proliferative cells and can divide after neuronal conversion. Indeed, S100β positive astrocytes were observed at different stages of cell division in the striatum at 30 days post NeuroD1+Dlx2 treatment (FIG. 7b-d). Similarly, with NeuN and Iba1 immunostaining, no significant changes were found in neuronal and microglia density nor the neuron/microglia ratio after astrocyte-to-neuron conversion (FIG. 8). Thus, neuronal and glial density are not altered after in vivo cell conversion.

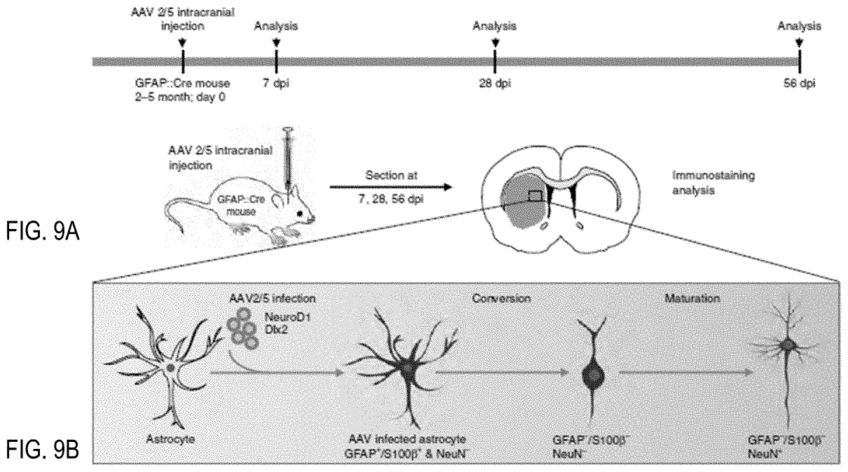

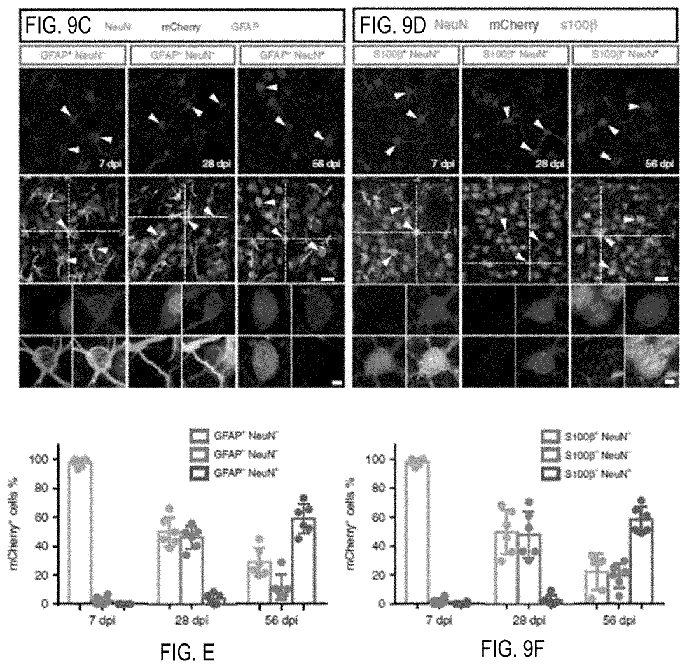

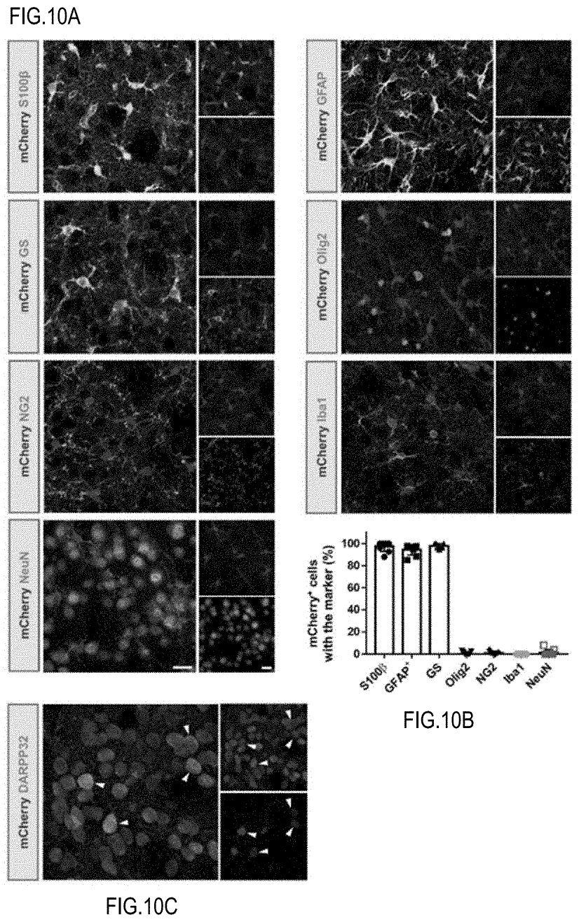

To further validate that the converted neurons originated from astrocytes, either AAV2/5 FLEx-mCherry alone as a control or AAV2/5 FLEx-NeuroD1-mCherry+FLEx-Dlx2-mCherry were injected into the striatum of GFAP::Cre transgenic mice (Cre77.6, Jackson Lab), in which Cre was expressed specifically in astrocytes (FIG. 9a, b). Control virus FLEx-mCherry expressed in astrocytes specifically in the Cre77.6 transgenic mouse brain (S100β positive, 97.4%, n=9 mice; GFAP positive, 94.3%, n=8 mice; GS positive, 97.8%, n=7 mice), rather than other types of glial cells or neurons (<5%, n=7 mice for each group; FIG. 10a, b). Only less than 2% of striatal neurons were labeled by mCherry in the control condition (n=9 mice; 3 mice were sacrificed at 28 dpi, and 6 mice were sacrificed at 58 dpi). Injection of NeuroD1+Dlx2 viruses into the striatum of Cre77.6 transgenic mice revealed a transitional conversion process at different time-points following viral infection. Specifically, mCherry positive cells in ND1+Dlx2 group showed astrocyte morphology at 7 dpi, with strong GFAP and S100β signal but no NeuN signal (FIG. 9c,d; left column). By 28 dpi, many mCherry positive cells lost GFAP and S100β signal but remained NeuN negative (GFAP negative & NeuN negative or S100β negative & NeuN negative), suggesting a transitional stage (FIG. 9c,d; middle column). At 56 dpi, the majority of mCherry positive cells became NeuN positive, suggesting the completion of the astrocyte-to-neuron conversion process (GFAP negative & NeuN positive or S100β negative & NeuN positive; FIG. 9c,d, right column). Quantification showed most of the mCherry positive cells were astrocytes (GFAP positive & NeuN negative: 97.8%, n=6 mice; S100β positive & NeuN negative: 98.1%, n=6 mice) at the beginning (7 dpi), then a number of the transient cells were observed at 28 dpi (GFAP positive & NeuN negative: 46.0%, n=6 mice; S100β positive & NeuN negative: 47.8%, n=6 mice), and an abundance of mCherry positive neurons were detected at 56 dpi (GFAP negative & NeuN positive: 59.1%, n=6 mice; S100β negative & NeuN positive: 58.2%, n=6 mice; FIG. 9e,f). Moreover, it was also found that most of the ND1+Dlx2 converted neurons in the striatum of Cre77.6 mice were DARPP32 positive MSNs (61.5±2.6%, n=8 mice; FIG. 10c). These results further demonstrate that striatal astrocytes can be reprogrammed into MSNs after ectopic expression of NeuroD1 and Dlx2.

Converting Striatal Astrocytes into GABAergic Neurons in the R6/2 Mouse Model

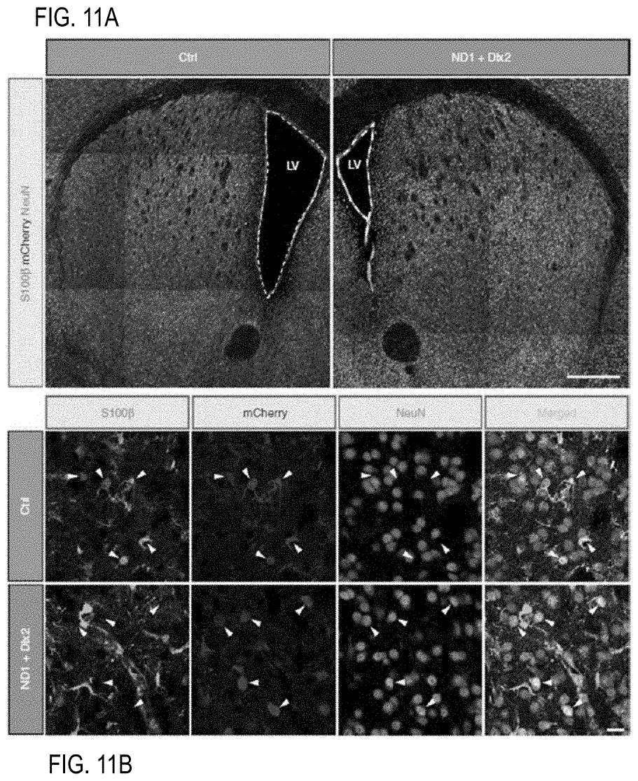

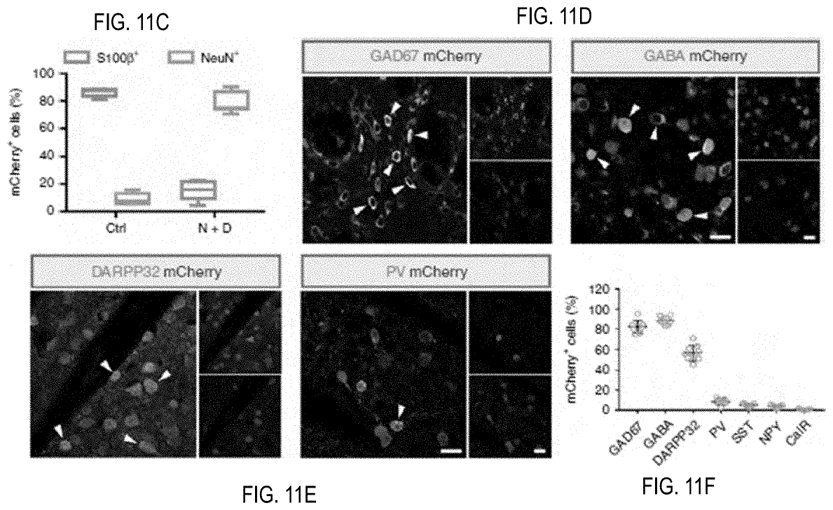

After testing successfully the conversion of striatal astrocytes into GABAergic neurons in the WT mice, it was next investigated whether this new approach can be used to regenerate GABAergic neurons in a mouse model of HD. The R6/2 transgenic mouse model for HD was employed, which has been well characterized in terms of the pathogenesis process and widely used for developing therapeutic interventions (Pouladi et al., Nat. Rev. Neurosci. 14:708-721 (2013)). To regenerate GABAergic neurons in the striatum of R6/2 mice, AAV2/5 NeuroD1 and Dlx2 were injected together into mice age of 2 months old (both female and male) when the HD mice started to show neurological phenotypes. One month after viral injection, in the mCherry control group, many infected cells (mCherry positive) with astrocyte-like morphology and that were immunopositive for S100β were observed (FIG. 11a, left panel; and FIG. 11b, top row); while NeuroD1+Dlx2 infected cells (mCherry positive) became immunopositive for NeuN (FIG. 11a, right panel; and FIG. 11b, bottom row). Quantified data showed that in the control group, 86.7% (n=6 mice) of mCherry positive cells were labeled by S100β, and only 9.2% of cells (n=6 mice) were labeled by NeuN (FIG. 11c). In NeuroD1+Dlx2 treated mice, 78.6% (n=7 mice) of viral infected cells were labeled by NeuN while only 15.3% of mCherry positive cells were labeled by S100β (FIG. 11c). Therefore, these results demonstrate that the striatal astrocytes in the R6/2 mouse brains also can be converted into neurons.

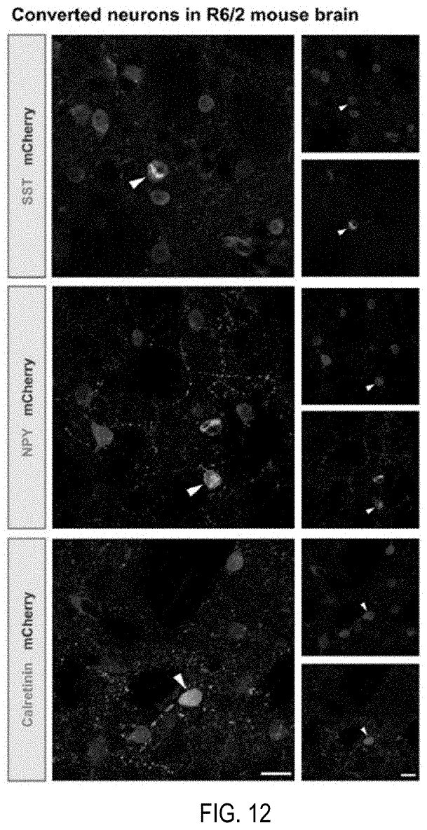

Next, mCherry was co-stained with a variety of GABAergic markers to determine what specific subtypes of GABAergic neurons were converted from astrocytes in R6/2 mouse striatum after injecting NeuroD1+Dlx2 AAV2/5 (38 dpi). It was found that the majority of astrocyte-converted neurons was immunopositive for GAD67 (82.4%, n=8 mice) or GABA (88.7%, n=8 mice; FIG. 11d, f), suggesting GABAergic neuron identity. Furthermore, 56.6% of the converted cells were DARPP32-positive MSNs (n=9 mice, FIG. 11e, f). There also were a few astrocyte-converted neurons immunopositive for PV (8.4%, n=9 mice; FIG. 11e, f), but they were rarely positive for SST, NPY, and CalR (all <5%; FIG. 11f and FIG. 12). These results demonstrate that ectopic expression of NeuroD1+Dlx2 in the striatal astrocytes of R6/2 mice can regenerate a significant number of MSNs for therapeutic treatment.

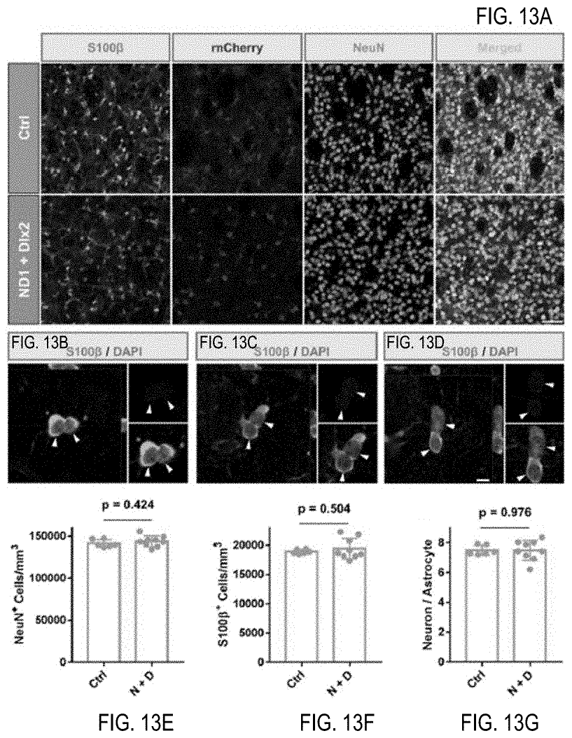

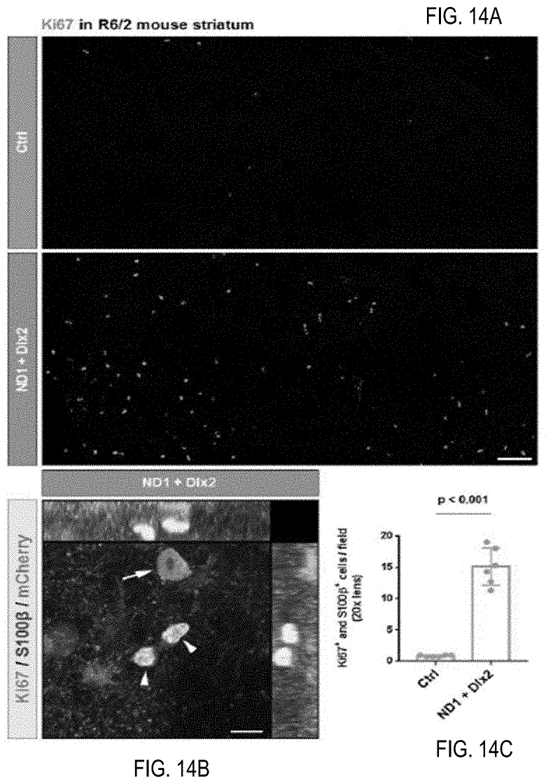

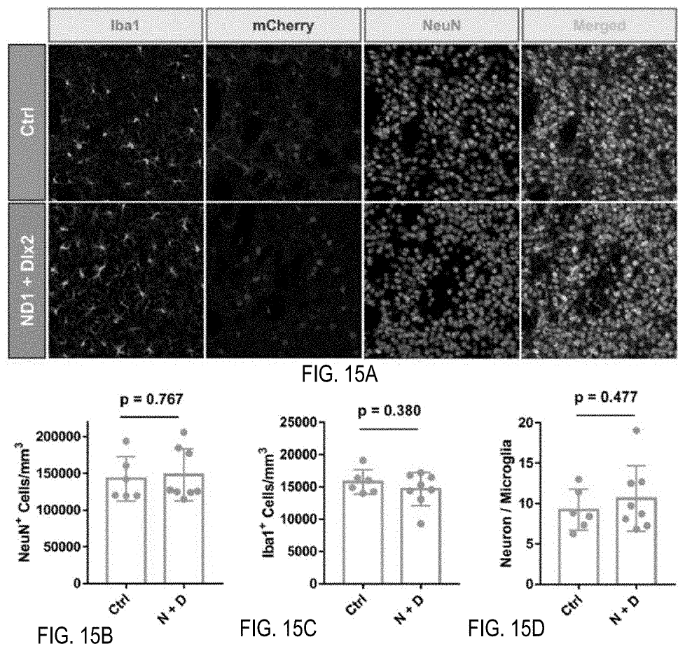

It was further investigated whether in vivo astrocyte-to-conversion could change the glial and neuronal density in the striatum of R6/2 mice. The cellular density of neurons and astrocytes as well as neuron/astrocyte ratio (FIG. 13) and neuron/microglia ratio (FIG. 15) were analyzed in R6/2 mice with or without cell conversion. Similar to the wild-type mouse striatum, no significant change was found in the cellular density nor the neuron/glia ratio in the striatum of R6/2 mice after in vivo cell conversion. A number of dividing astrocytes in the R6/2 mouse striatum after NeuroD1+Dlx2 treatment were also observed (FIG. 13b-d), suggesting that the astrocyte-to-neuron conversion may stimulate proliferation of astrocytes. To test this possibility, the Ki67-labeled dividing astrocytes were compared between control and NeuroD1+Dlx2 group in R6/2 mouse striatum (30 dpi). It was found that compared with the control group, the number of Ki67 positive astrocytes in NeuroD1+Dlx2 group was significantly increased by ˜15-fold (p<0.001, unpaired Student's t-test; FIG. 14). These data suggest that in vivo cell conversion facilitates astrocytic proliferation, explaining why astrocytes are never depleted in the converted areas.

### Functional Analysis of Converted Striatal Neurons in the R6/2 Mouse Brain

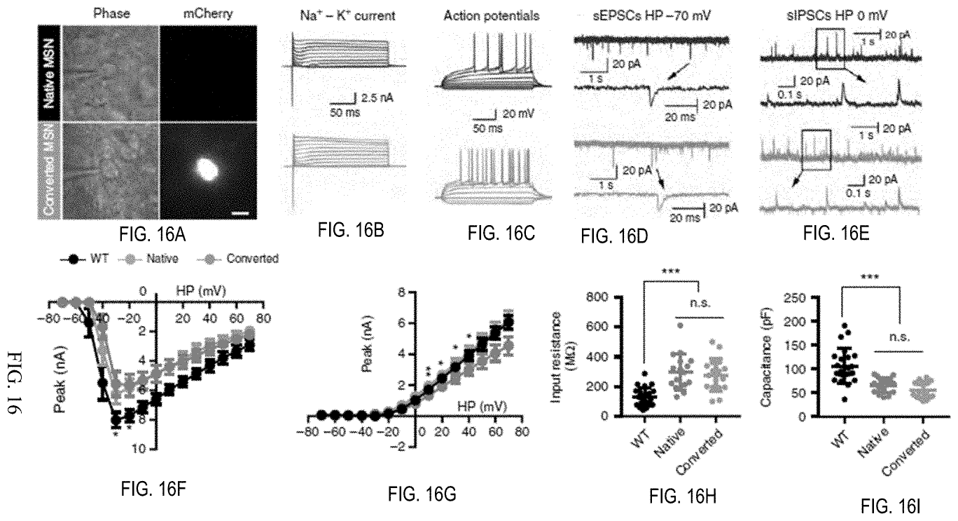

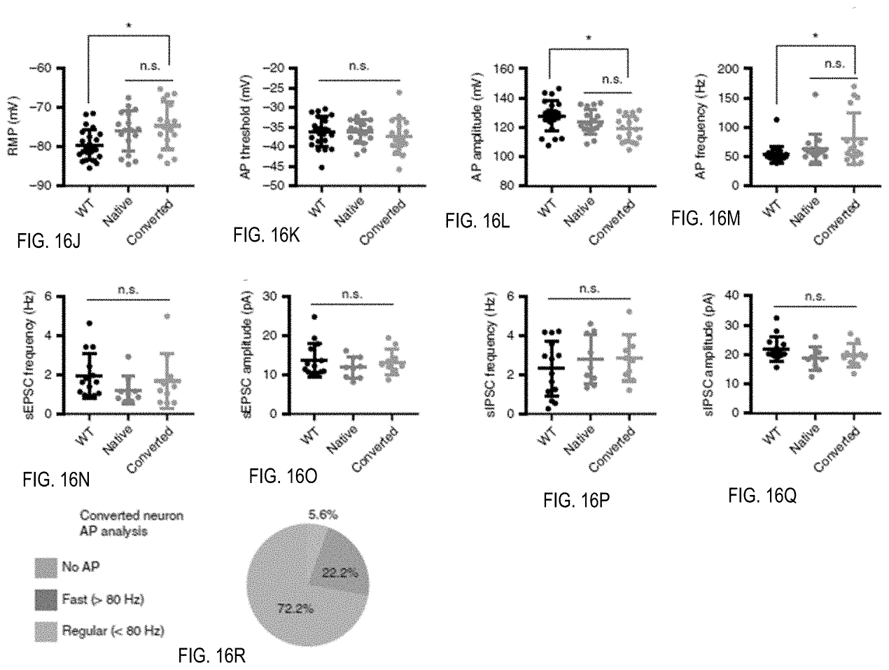

The functional properties of astrocyte-converted neurons (mCherry positive; FIG. 16a) in comparison to the native neurons (mCherry-; FIG. 16a) were assessed using whole-cell recordings in acute striatal slices from R6/2 mice at 30-32 dpi following AAV infection. The Na positive K positive currents (FIG. 16b-g) were compared and it was found that there was no significant difference between Na positive currents of converted and neighboring non-converted neurons in R6/2 mice, but both Na positive and K positive currents were significantly smaller than that recorded in WT mice (FIG. 16f). For K positive currents, converted neurons showed similar amplitude to the WT neurons, while the non-converted neurons of R6/2 mice showed slightly smaller amplitude (FIG. 16g; n=15 neurons/group from 3 mice). Next, for action potential firing, it was found that 17 out of 18 mCherry positive cells, and 17 native neurons, were able to fire repetitive action potentials when evoked by step current injection (FIG. 16c, a total of 35 cells from 3 mice were recorded). Regarding basic electrical properties such as the cell membrane input resistance, cell membrane capacitance, resting membrane potential (RMP), action potential (AP) threshold, AP amplitude, and AP frequency, no significant differences were found between native and converted neurons (FIG. 16h-m). When compared with the striatal neurons in the WT mice, the converted neurons in the R6/2 mice had higher input resistance, lower cell capacitance, lower resting membrane potential, and lower action potential amplitude (FIG. 16h-m), suggesting that at 1 month after conversion, these newly converted neurons have not fully matured yet.

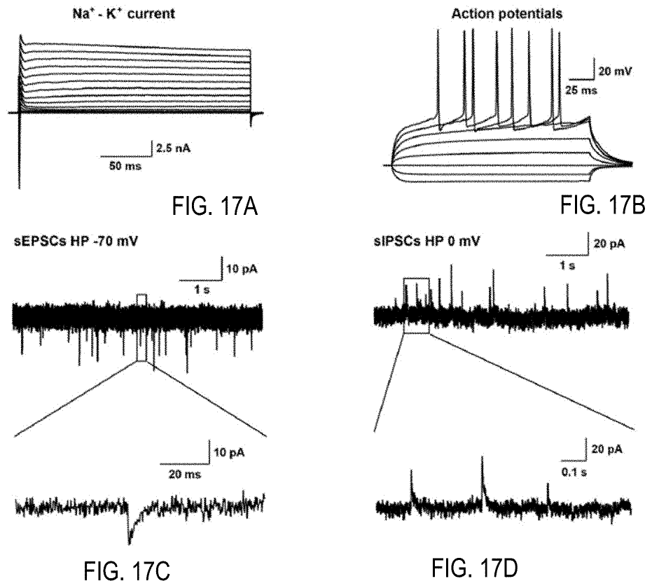

Different subtypes of GABAergic neurons have distinct AP firing pattern characteristics. When analyzing the AP firing pattern of the astrocyte-converted neurons, excluding the single mCherry positive cell incapable of firing an AP, most of the converted neurons (72.2%) showed a regular firing frequency (<80 Hz, n=13) with a long delay to the initial AP spike upon stimulation (FIG. 16c-r), consistent with a typical MSN firing pattern in the striatum. It also was found that 22.2% of converted neurons displayed a fast firing frequency (>80 Hz, n=4; FIG. 16r), consistent with a typical PV neuron firing pattern. Moreover, whether astrocyte-converted neurons could be incorporated in local synaptic circuits was investigated by examining spontaneous postsynaptic currents (sPSCs), which represent functional synaptic inputs to the converted neurons. As shown by the representative traces (FIG. 16d, e), both spontaneous excitatory postsynaptic currents (sEPSCs) and spontaneous inhibitory postsynaptic currents (sIPSCs) were detected in all native neurons (n=9 from 3 mice) and converted neurons (n=11 from 3 mice). Furthermore, quantitative analyses found no significant differences in the frequency and amplitude of both sEPSCs and sIPSCs among the native neurons and converted neurons in the R6/2 mice (FIG. 16n-q) as well as the striatal neurons in the WT mice (FIG. 17c, d). Together, the electrophysiological analyses suggest that striatal astrocytes in the R6/2 mouse brain can be converted into typical functional GABAergic neurons, which can further integrate into local synaptic circuits.

### Axonal Projections of the Astrocyte-Converted Neurons

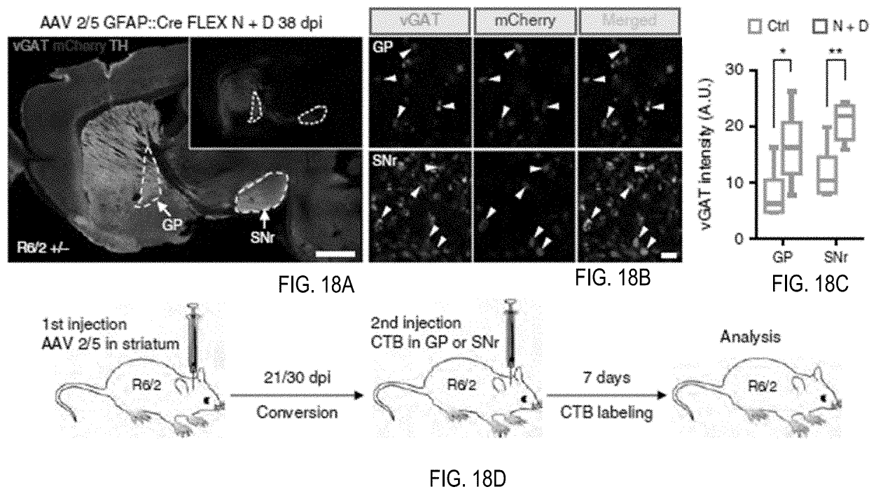

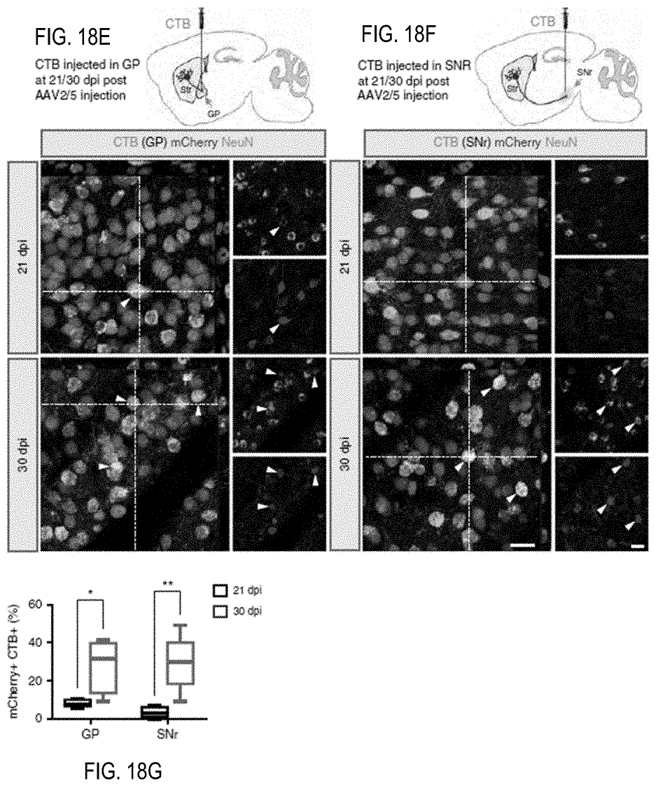

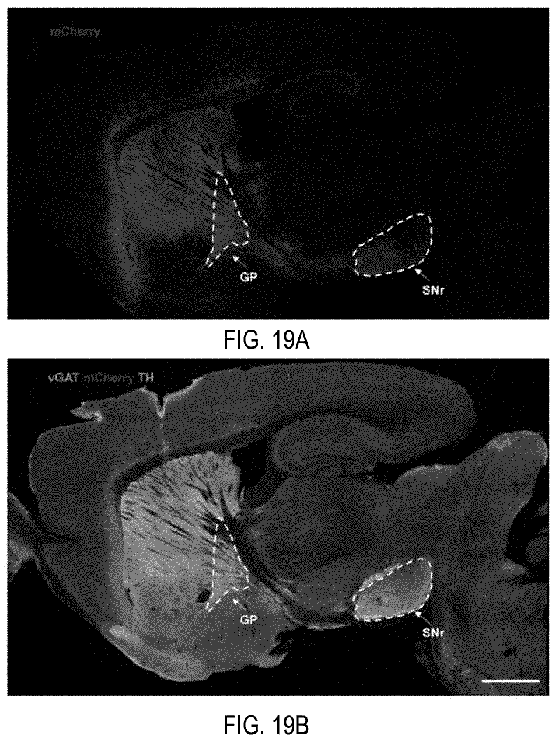

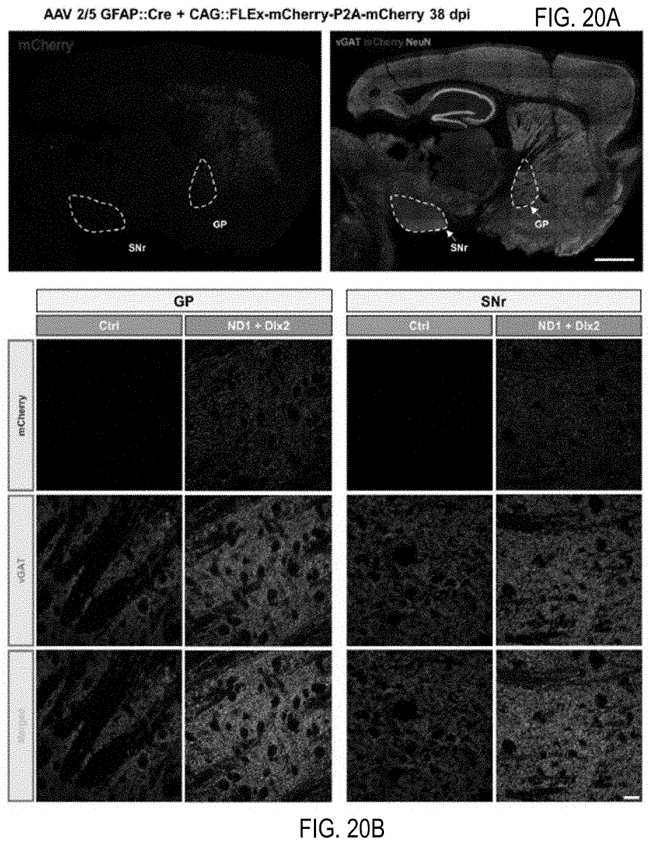

Striatal MSNs send axonal projections to two distinct nuclei within the basal ganglia, the external globus pallidus (GP) and the substantia nigra pars reticulata (SNr). Due to the severe loss of MSNs in the striatum, these two output pathways are severely disrupted in the HD brain. It was therefore investigated whether the astrocyte-converted neurons in the striatum could send their axonal projections into these distal targets. Indeed, a clear mCherry positive axonal tract extending from the striatum to the GP and SNr was found in NeuroD1+Dlx2 treated R6/2 mice (FIG. 18a; and FIG. 19), but such mCherry positive axonal tract was not detected in the control mice (FIG. 20a). Further immunostaining showed that the mCherry positive puncta (axonal nerve terminals) in both the GP and SNr were co-labeled with vGAT, a marker of pre-synaptic GABAergic nerve terminals (FIG. 18b). Quantified data showed that the intensity of the vGAT in the GP and SNr were significantly increased in NeuroD1+Dlx2 treated R6/2 mouse brains (FIG. 18c and FIG. 20b). These findings demonstrate that the astrocyte-converted neurons can send out GABAergic nerve projections and strengthen GABAergic outputs from the striatum to the GP and SNr in the R6/2 mouse brain.

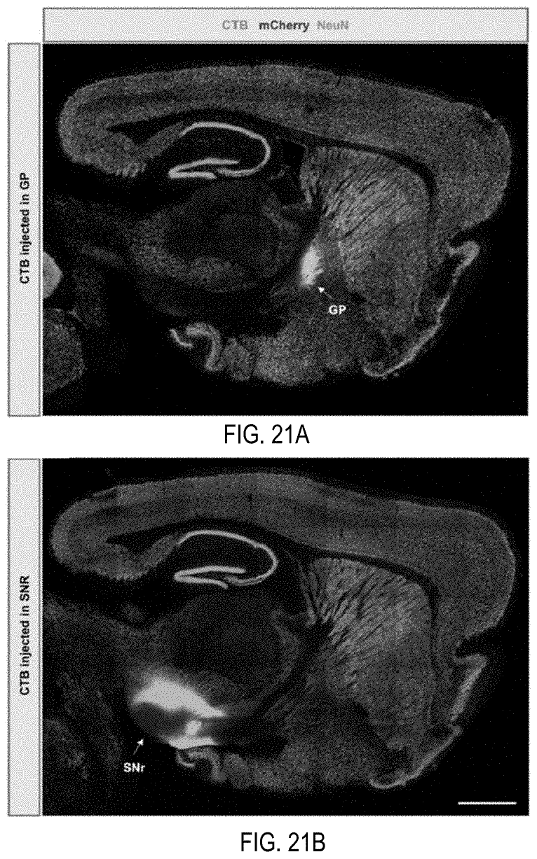

To further investigate the progress of axonal projections after NeuroD1+Dlx2 induced in vivo conversion in the R6/2 mouse brain, a retrograde tracer, cholera toxin subunit B (CTB), was injected into the GP or SNr at two different time points, 21 dpi or 30 dpi. At 7 days post CTB injection, the mice were sacrificed for analysis of the CTB-labeled neurons in the striatum (see schematic illustration in FIG. 18d). Sagittal brain sections were made for validating the CTB injected sites (FIG. 21). When CTB was injected at 21 dpi, a number of CTB-labeled native neurons (NeuNpositive, mCherry negative) was found in the striatum, but very few of the converted neurons (NeuN positive, mCherry positive) were labeled by CTB (GP=8.2%, n=509 from 5 mice; SNr=3.5%, n=483 from 5 mice; FIG. 18e-g). However, when CTB was injected at 30 dpi, it was found that CTB was not only detected in native neurons but also in converted neurons (FIG. 18e, f). Quantified data showed that the percentage of CTB-labeled converted neurons was significantly increased when CTB was injected at 30 dpi compared to 21 dpi (GP=27.7%, n=535 from 5 mice, p=0.014; SNr=29.4%, n=511 from 5 mice, p=0.004, unpaired Student's t-test; FIG. 18g). Therefore, these data demonstrate that the in vivo converted MSNs can extend their axonal projections into the GP and SNr in the R6/2 mouse brain.

### Alleviation of Neurodegeneration in R6/2 Mice by In Vivo Cell Conversion

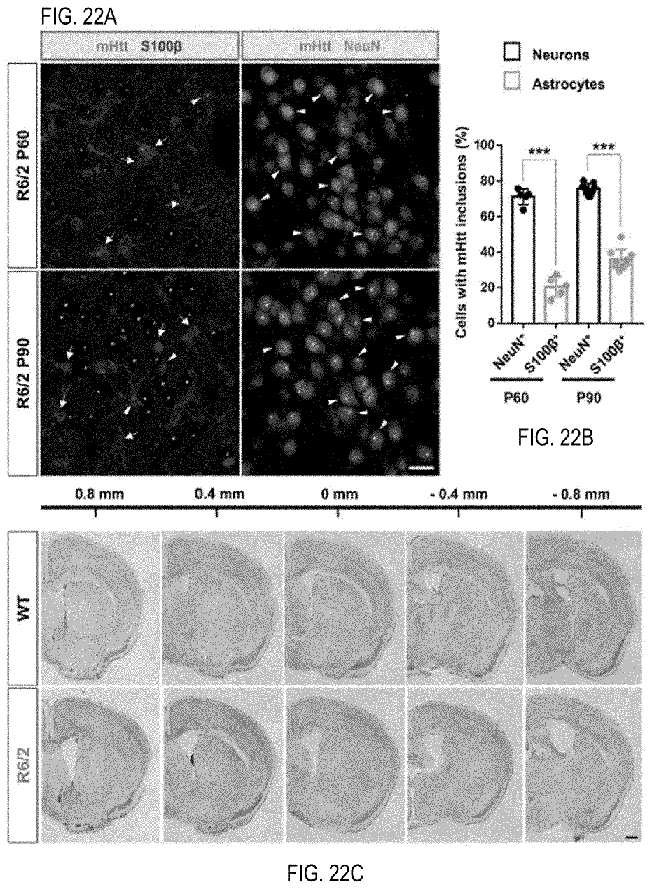

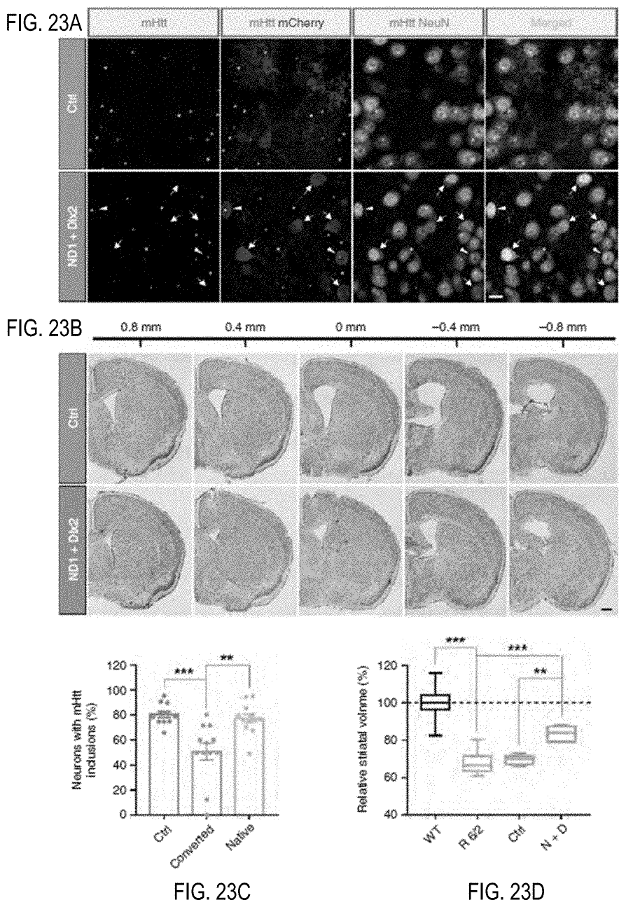

Huntington's disease is an autosomal dominant disorder associated with a mutation in the gene encoding huntingtin (Htt). The mutation leads to excessive polyglutamine repeats yielding mutant Htt (mHtt), which misfolds causing aggregation and subsequent neurodegeneration, particularly in the striatum. The mHtt aggregation (inclusion) within the converted neurons was investigated. Because the newly generated neurons are converted from astrocytes and mHtt aggregation has been detected both in neurons and astrocytes in R6/2 mouse striatum, the progress of mHtt inclusions in striatal astrocytes and neurons was compared at age 60 days (P60) and 90 days (P90) in the R6/2 mouse striatum. It was found that mHtt nuclear inclusions were detected at P60 in 20.6% of 510013 positive astrocytes and 71.1% in neurons (FIG. 22a). At 3 months old, 35.8% astrocytes and 75.5% of neurons displayed mHtt inclusions (FIG. 22b). These data suggest that astrocytes have less mHtt inclusions than neurons in the R6/2 mouse striatum. Interestingly, it was found that the astrocyte-converted neurons (51.1%, n=151 neurons from 12 mice) displayed less mHtt inclusions when compared to the native neurons (77.1%, n=655 neurons from 12 mice; p<0.002, One-way ANOVA with Bonferroni's post-hoc test), or the neurons in the control group (80.3%, n=709 neurons from 11 mice; p<0.001, One-way ANOVA with Bonferroni's post-hoc test) (FIG. 23a, c). These results indicate that in the R6/2 mouse striatum, neurons have more mHtt nuclear inclusions than astrocytes and the astrocyte-converted neurons have less mHtt nuclear inclusions than preexisting neurons.

Striatum atrophy caused by neurodegeneration has been reported previously in the R6/2 mouse brain (Paul et al., Nature 509:96-100 (2014)). The relative striatum volume between R6/2 and wild type (WT) littermates was examined. Obvious striatal atrophy was observed in R6/2 mice compared to their WT littermates (FIG. 22c). Quantification data showed a 31.8% reduction in the striatum volume in 3-month-old R6/2 mice (n=9 mice, p<0.001, One-way ANOVA with Bonferroni's post-hoc test; FIG. 23d). It was found that the striatum atrophy was alleviated in the NeuroD1+Dlx2-treated R6/2 mice compared to the control virus-treated R6/2 mice (FIG. 23b; AAV2/5 were injected at P60, mice were sacrificed at P98). Quantified data showed 30.3% striatum atrophy in the control virus-treated group (n=6 mice), but only 16.9% striatum atrophy in the NeuroD1+Dlx2 group (n=7 mice, p=0.004, One-way ANOVA with Bonferroni's post-hoc test; FIG. 23d). Therefore, these results suggest that the in vivo astrocyte-to-neuron conversion approach can reduce the striatum atrophy in R6/2 mice.

### Attenuation of Phenotypic Deficits in R6/2 Mice by In Vivo Cell Conversion

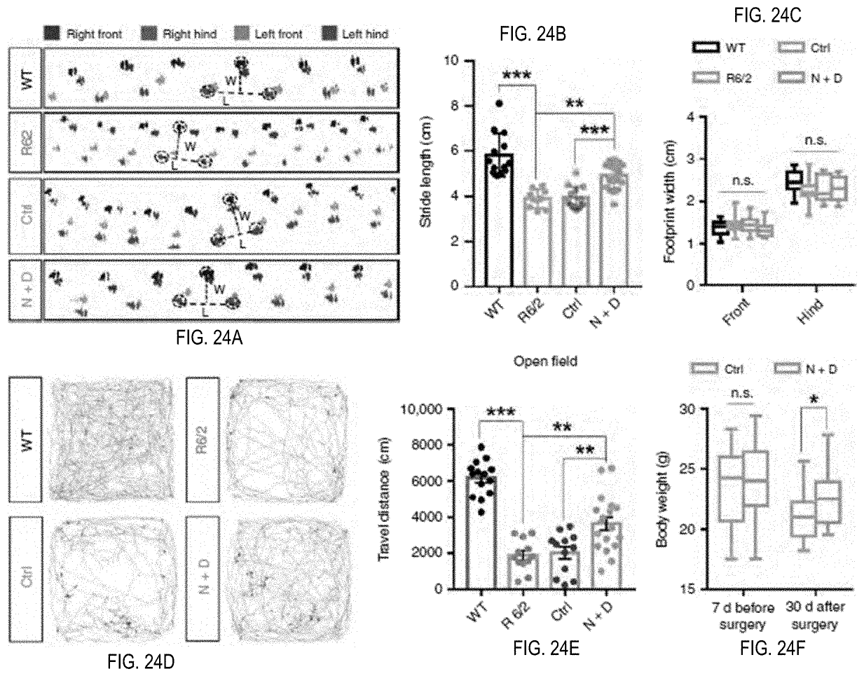

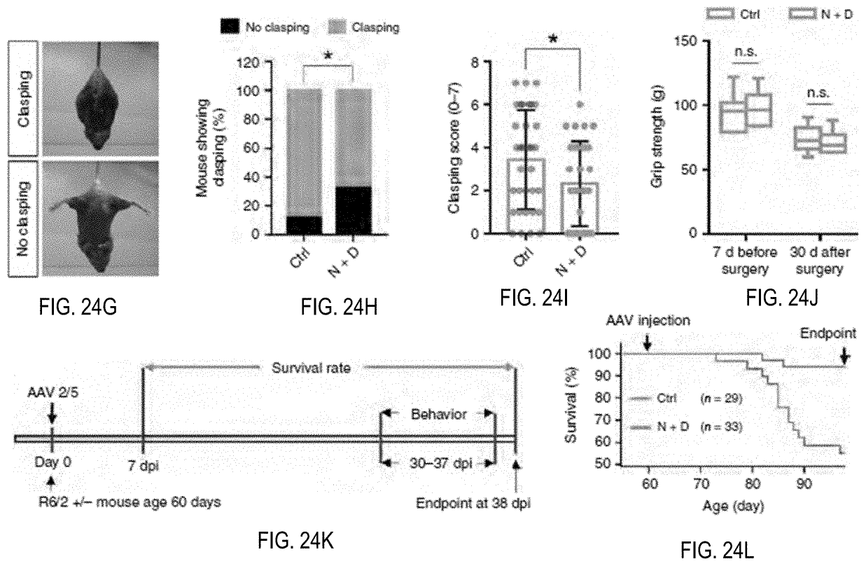

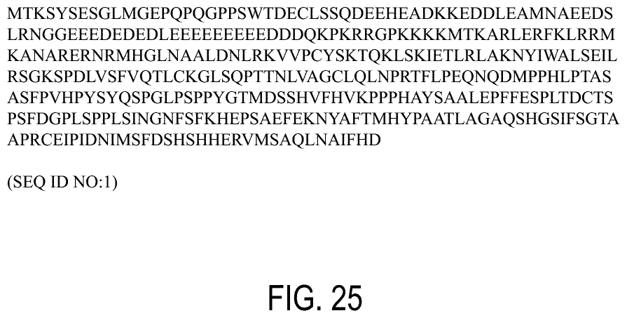

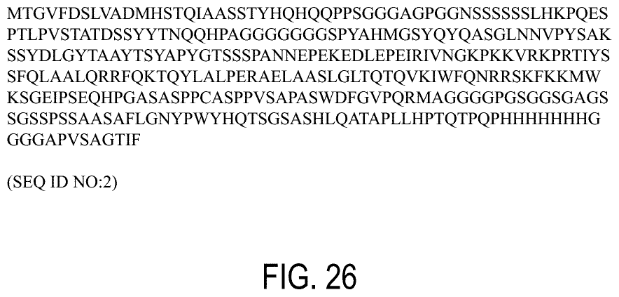

The R6/2 mice display a progressive neurological phenotype that mimics many of the features of HD patients. Whether the in vivo cell conversion approach could alleviate the abnormal phenotypes in the R6/2 mice was examined using a series of behavioral tests. The catwalk behavioral test was performed to evaluate the gait changes in the R6/2 mice in comparison to their WT littermates (P90-97). It was found that the average stride length was significantly reduced in the R6/2 mice when compared to WT littermates (WT=5.80±0.30 cm, n=13 mice, 6 male and 7 female; R6/2=3.91±0.11 cm, n=10, 3 male and 7 females; p<0.001, One-way ANOVA with Bonferroni's post-hoc test; FIG. 24a, b). To test the effect of gene therapy, R6/2 mice received intracranial AAV2/5 injection bilaterally at P60 and after 30-37 days post viral injection underwent the catwalk behavioral test (FIG. 24k). It was found that the stride length was significantly improved in the NeuroD1+Dlx2 treated mice (4.91±0.13 cm, n=19, 8 males and 11 females; p<0.001, One-way ANOVA with Bonferroni's post-hoc test), compared to the control AAV2/5 mCherry-injected mice (3.95±0.14 cm, n=13, 6 males and 7 females; FIG. 24a, b). There was no significant difference in footprint width between different groups (FIG. 24a, c). The locomotion activity was assessed with the open field test. It was found that the total travel distance of R6/2 mice (in 20 minutes) showed a dramatic decrease (1886±252 cm, n=12, 5 males and 7 females; p<0.001, One-way ANOVA with Bonferroni's post-hoc test) compared to the WT littermates (6163.8±263.0 cm, n=14, 7 males and 7 females; FIG. 24d, e). The walking distance showed a significant increase in the NeuroD1+Dlx2-treated R6/2 mice (3648±367 cm, n=18 mice, 10 male and 8 female mice), compared to the mCherry-treated R6/2 mice (2023±331 cm, n=12 mice, 5 male and 7 female mice; One-way ANOVA with Bonferroni's post-hoc test; FIG. 24d, e). These results suggest that the in vivo cell conversion approach significantly improves the motor functions of the R6/2 mice.

In addition, the body weight, clasping behavior, and grip strength of the R6/2 mice after gene therapy treatment was examined. R6/2 mice have been reported to lose body weight at 8 weeks old (Menalled et al., Neurobiol. Dis. 35:319-336 (2009)). To test the gene therapy effects, the R6/2 mice were randomly divided into two groups, and the body weight was measured 7 days before surgery. No significant difference was found between the two groups (p=0.367; FIG. 240. The R6/2 mice that were treated with NeuroD1+Dlx2 lost less body weight than the R6/2 mice that were injected with the control virus at 30 dpi (Ctrl=21.13±0.39 g, n=25, 9 females and 16 males; N+D=22.42±0.38 g, n=28, 11 females and 17 males; p=0.021, unpaired Student's t-test; FIG. 240. Next, the paw clasp test was used to measure dystonia and dyskinesia in the R6/2 mice. The typical clasping phenotype (FIG. 24g, top panel) was observed in most of the R6/2 mice. However, the percentage of R6/2 mice showing clasping was significantly reduced after NeuroD1+Dlx2 treatment (Ctrl=88.2%, n=34, 14 females and 20 males; N+D=67.7%, n=31, 13 females and 18 males; p=0.045, 2-sided Pearson Chi-Square test; FIG. 24h). Moreover, the clasping score also was significantly decreased in NeuroD1+Dlx2 group (Ctrl=3.4±0.4, n=34, 14 females and 20 males; N+D=2.3±0.4, n=31, 13 females and 18 males; p=0.040, unpaired Student's t-test; FIG. 24i). Grip strength was measured and it was found that there was no significant difference between the control virus-treated and the NeuroD1+Dlx2 treated R6/2 mice (FIG. 24j). Remarkably, when the survival rate of R6/2 mice was analyzed at 38 dpi (viral injection at 2-month-old), 93.9% of the R6/2 mice that were injected with NeuroD1+Dlx2 were still alive, but 44.8% of the R6/2 mice that received control AAV2/5 mCherry injection were dead, which is expected for R6/2 mice at this age (P<0.001, 2-sided Pearson Chi-Square test; FIG. 24I). Altogether, these results demonstrate that in vivo regeneration of GABAergic neurons in the striatum of R6/2 mice can partially rescue the phenotypic deficits and extend the life expectancy.

### Methods and Materials

### Animals

Animals were housed in a 12:12 hour light:dark cycle with free access to chow and water. The R6/2 strain (B6CBA-Tg(HDexon1)62Gpb/3J) was maintained by ovarian transplant hemizygote females x B6CBAF1/J males, both were purchased from Jackson Laboratory. Mice were genotyped by PCR after weaning (P21-27) and the littermates without mutation were used as normal mice (2-5 months). Some of the R6/2 transgenic mice were directly purchased from the Jackson Laboratory at ages of 4-6 weeks. The GFAP::Cre transgenic mice (B6.Cg-Tg(Gfapcre) 77.6Mvs/2J, Cre77.6) were purchased from Jackson Laboratory as well. The 2-5 months old hemizygous mice were used for AAV injection. Both male and female mice were used in this study. Experimental protocols were approved by the Pennsylvania State University IACUC and in accordance with guidelines of National Institutes of Health.

### AAV Production

Recombinant AAV2/5 was produced in 293 AAV cells (Cell Biolabs). Briefly, polyethylenimine (PEI, linear, MW 25,000) was used for transfection of triple plasmids: the pAAV expression vector, pAAV5-RC (Cell Biolab) and pHelper (Cell Biolab). At 72 hours post transfection, cells were harvested and centrifuged. The cells were then cyclically frozen and thawed four times by placing it on dry ice/ethanol and a 37° C. water bath. AAV crude lysate was purified by centrifugation at 54,000 rpm for 1 hour in discontinuous iodixanol gradients with a Beckman SW55Ti rotor. The virus-containing layer was extracted and concentrated by Millipore Amicon Ultra Centrifugal Filters. The AAV2/5 genome copies (GC) per injection for GFAP::Cre is 3.55×10⁷ GC; for CAG::FLEx-mCherry-P2A-mCherry, it is 2.54×10⁹ GC; for CAG::FLEx-NeuroD1-P2AmCherry, it is 1.59×10⁹ GC; and for CAG::FLEx-Dlx2-P2A-mCherry, it is 2.42×10⁹ GC. Virus titer was 7.7×10¹⁰ GC/mL for GFAP::Cre; 1.65×10¹² GC/mL for FLEx-mCherry-P2A-mCherry; 2.07×10¹² GC/mL for FLEx-NeuroD1-P2A-mCherry, and 3.14×10¹² GC/mL for FLEx-Dlx2-P2AmCherry, determined by QuickTiter™ AAV Quantitation Kit (Cell Biolabs).

### Stereotaxic Viral Injection

Brain surgeries were conducted on 2-5 month-old wild type mice or 2 month-old R6/2 mice for AAV injection. The mice were anesthetized by injecting ketamine/xylazine (120 mg/kg and 16 mg/kg) into the peritoneum, followed by fur trimming, and placement into a stereotaxic setup. Artificial eye ointment was applied to cover the eye for protection purposes. Oxygen was provided for the R6/2 mice throughout surgery. The operation began with a midline scalp incision followed by the creation of a (˜1 mm) drill hole on the skull for intracranial injection into the striatum (AP +0.6 mm, ML±1.8 mm, DV −3.5 mm). Each mouse received a bilateral injection of AAV2/5 using a 5 μL syringe and a 34 G needle. The injection volume was 2 μL and the flow rate was controlled at 0.2 μL/minute. Some R6/2 mice received secondary surgery after AAV2/5 injection where CTB (ThermoFisher, C34775) was delivered. The mice were anesthetized by 2.5% Avertin (250-325 mg/kg), and oxygen was supplied during the surgery. CTB (0.5 μg/site) was injected into the globus pallidus (AP −0.2 mm, ML 1.8 mm, DV −4.0 mm) or substantia nigra pars reticulata (AP −3.0 mm, ML 1.7 mm, DV −4.0 mm), two target areas of the striatal MSN's projections. After viral injection, the needle was kept in place for at least 10 minutes before being slowly withdrawn. Coordinates are measured from bregma.

### Immunohistochemistry and Analysis

For brain slice immunostaining, the animals were deeply anesthetized with 2.5% Avertin and then quickly perfused with ice-cold artificial cerebrospinal fluid (aCSF) to wash away the blood. Then brains were quickly removed and post-fixed in 4% PFA overnight at 4° C. in darkness. After fixation, the samples were cut into 40 μm sections by a vibratome (Leica, VTS1000). Brain slices were washed three times in phosphate buffer solution (PBS, pH: 7.35, OSM: 300) for ten minutes each. Blocking was performed for 2 hours in 0.3% triton PBS+5% normal donkey serum (NDS). Primary antibody was diluted in 0.05% triton PBS+5% NDS and incubated in a moist environment at 4° C. for two nights (see Table 2 for the primary antibody information). After washing three times in PBS, the samples were incubated with appropriate secondary antibodies conjugated to Alexa Flour 405, or Alexa Flour 488, or Cy3, or Alexa Flour 647 (1:500, Jackson ImmunoResearch) for 2 hours at room temperature, followed by extensive washing in PBS. The secondary antibody was diluted in 0.05% triton PBS+5% NDS. For GAD67 and GABA immunostaining, the samples were fixed in 4% PFA and 0.2% glutaraldehyde, the sections were mildly permeabilized in 0.05% Triton PBS for 30 minutes, and Triton was removed for rest of the immunostaining procedure. The samples were mounted on glass slides and stored at 4° C. in darkness.

TABLE 2
Antibodies used.

| Antibodies (dilution) | Host | Source | Catalog # |
|---|---|---|---|
| RFP (1:1000) | Rat mAb | Chromotek | Sf8-100 |
| NeuroD1 (1:1000) | Mouse mAb | Abcam | AB60704 |
| Dlx2 (1:1000) | Rabbit | Abcam | AB30339; |
| | | | discontinued |
| Dlx2 (1:200) | Rabbit | Millipore | AB5726 |
| Cre (1:1000) | Mouse mAb | Millipore | MAB3120 |
| GFAP (1:2000) | Rabbit | Millipore | AB5804 |
| GFAP (1:1000) | Chicken | Millipore | AB5541 |
| Glutamine synthetase | Mouse mAb | Millipore | MAB302 |
| (1:1000) | | | |
| S100β (1:1000) | Rabbit | Abcam | ab52642 |
| NG2 (1:150) | Mouse | Abcam | ab50009 |
| Olig2 (1:1000) | Rabbit | Millipore | AB9610 |
| Iba1 (1:1000) | Rabbit | Wako | 019-19741 |
| NeuN (1:2000) | Guinea Pig | Millipore | ABN90 |
| NeuN (1:2000) | Rabbit | Millipore | ABN78 |
| GAD67 (1:1000) | Mouse mAb | Millipore | MABS406 |
| GABA (1:1000) | Rabbit | Sigma | A2052 |
| DARPP32 (1:1000) | Rabbit | Millipore | AB10518 |
| Parvalbumin (1:5000) | Mouse mAb | Sigma | P3088 |
| Somatostatin (1:300) | Rat | Millipore | MAB354 |
| NPY (1:2000) | Rabbit | Abcam | AB30914 |
| Calretinin (1:2000) | Goat | Millipore | AB1550 |
| vGAT (1:500) | Guinea Pig | SYSY | 131004 |
| mHtt (1:1000) | Mouse mAb | DSHB | MW7 |
| Ki67 (1:500) | Rabbit | Abcam | Ab15580 |
| S100β (1:1000) | Mouse mAb | Abcam | Ab66028 |

The images were acquired by a Zeiss confocal microscope (LSM 800). For quantification, 2-6 regions in the striatum were randomly selected for confocal imaging (20× lens 2-4 regions; 40× lens 4-6 regions). Most imaging analysis was performed with Zeiss software ZEN. In order to avoid the impact of human bias on the analysis, some of the mouse information was blinded during confocal imaging. Moreover, image analysis was further performed blindly: the person who did the quantification did not know the injected virus info. After quantification, another person decoded the mouse information. The Image J software was used for quantifying the intensity of vGAT.

### Electrophysiology

Brain slices were prepared at 30-32 days after AAV injection, and cut to 300 μm thick coronal sections with a vibratome (Leica, VTS1200) at room temperature in cutting solution (in mM: 93 NMDG, 93 HCl, 2.5 KCl, 1.25 NaH₂PO₄, 30 NaHCO₃, 20 HEPES, 15 Glucose, 12 N-Acetyl-L-cysteine, 5 Sodium ascorbate, 2 Thiourea, 3 Sodium pyruvate, 7 MgSO₄, 0.5 CaCl₂, pH 7.3-7.4, 300 mOsmo, solution was bubbled with 95% O₂/5% CO₂). Then, slices were transferred to holding solutions with continuous 95% O₂/5% CO₂ bubbling (in mM): 92 NaCl, 2.5 KCl, 1.25 NaH₂PO₄, 30 NaHCO₃, 20 HEPES, 15 Glucose, 12 N-Acetyl-L-cysteine, 5 Sodium ascorbate, 2 Thiourea, 3 Sodium pyruvate, 2 MgSO₄, and 2 CaCl₂. After 0.5-1 hour recovery, the slices were transferred to a chamber for electrophysiology study. The recording chamber was filled with artificial cerebral spinal fluid (ACSF) containing:

119 mM NaCl, 2.5 mM KCl, 26 mM NaHCO₃, 1.25 mM NaH₂PO₄, 2.5 mM CaCl₂, 1.3 mM MgCl₂ and 10 mM glucose, and constantly bubbled with 95% 02 and 5% CO₂ at 32-33° C. Whole-cell recordings were conducted using a pipette solution consisting of 135 mM K-Gluconate, 5 mM Naphosphocreatine, 10 mM KCl, 2 mM EGTA, 10 mM HEPES, 4 mM MgATP, and 0.5 mM Na₂GTP (pH 7.3, adjusted with KOH, 290 mOsm/L). To record the spontaneous synaptic events, the potassium gluconate in the pipette solution was replaced with Cs-methanesulfonate to block K positive channels and reduce noise. Pipette resistance was typically 4-6 MΩ, and series resistance was around 20-40 MΩ The membrane potential was held at −70 mV for sEPSC recording, and at 0 mV for sIPSC recording. Data were collected using pClamp 9 software (Molecular Devices, Palo Alto, Calif.), sampled at 10 kHz, and filtered at 1 kHz, then analyzed with pClamp 9 Clampfit and MiniAnalysis software (Synaptosoft, Decator, Ga.).

### Nissle Staining and Quantification of Relative Striatum Volume

To assess striatal atrophy, brains were sliced and collected in a serial manner allowing accurate identification of the anterior/posterior sections relative to the bregma so that the striatal volume could be calculated. Every 5th section (anterior and posterior of bregma) covering the entire striatum was included for calculating the striatum volume. Samples were mounted on glass slides and allowed to dry at room temperature for 24 hours and then stained with crystal violet. The stained sections were photographed by Keyence microscope (BZ9000). Striatum area was outlined according to the mouse brain atlas and the size of the striatum was blindly measured by Image J software. Striatal volume was calculated using Cavalieri's principle (volume=s1d1+s2d2+ . . . +sndn s, s is surface area and d is the distance between two sections). All of the values were normalized to the striatal volume in wild type littermates.

### Behavioral Tests and Analyses

The mice were acclimated to the behavioral testing room for one hour in order to reduce the effect of the stress associated with movement of the cages. Both female and male mice were included for behavioral tests, and the female and male mouse number was stated in the results section.

Catwalk. The CatWalk XT 10.6 (Noldus) system was used to analyze gait deficits in R6/2 mice. The stride length and footprint width were analyzed to evaluate the treatment effects of in vivo cell conversion. The maximum run duration was 6 seconds, with a maximum speed variation of 60% in order to reduce variability in the mouse's natural gait pattern. Three compliant trials were acquired per mouse in order to ensure reproducibility. Before each trial the walkway was cleaned with 70% ethanol and dried, then fanned in order to reduce any remaining alcohol odor. During the trial period the room light was turned off. The mouse gait was analyzed automatically by the system software (CatWalk XT 10.6, Noldus). To avoid detections of false footprint, such as mouse excrement, nose-point, tail, and belly, the analysis results were further checked visually and corrected blindly.

Open field test. The open field test was used to assess the locomotion activity in the R6/2 mice. The study arena was a white open-top box (50×50×30 cm³), and the mouse was gently placed in the center to start the test. The computer program (EthoVision XT Version 8, Noldus) was calibrated to the arena and set to track center-point, nose-point, and tail-point of the mouse using dynamic subtraction. The mouse freely moved in the open box for 20 minutes, and its route was automatically tracked and analyzed by the software (Ethovision XT Version 8).

Clasping. The clasping test was used to measure dystonia and dyskinesia. The mouse was suspended upside down by its tail for 14 seconds. The 14-second trial was split into seven intervals, with 2-second for each interval. The animal was awarded a score of 0 (no clasping) or 1 (clasping). The score for the seven intervals was summed for each mouse allowing a maximum score of 7. Clasping was defined as a behavior whereby paws crossed and came to the chest for any period of time within each 2-second interval. The test was video recorded and analyzed later in a blind fashion.

Mouse weight. The mouse weight was tracked in order to observe any severe weight loss, as the R6/2 mouse model is known to have up to 20% weight decrease after 3 months of age. The mice were weighed individually each Tuesday at 5:00 PM in the animals' homeroom inside an approved vent hood.

Grip Strength. The grip strength test was used to quantitatively measure the strength of the mouse forepaws. The grip strength meter (BIO-GS3, Bioseb) was set to record in grams. Each mouse was held by its tail and allowed to grasp the metal grid with only its two front paws. The mouse was pulled until failure to record the maximum strength for each trial. Each mouse was tested three times per time point and the three trials were then averaged to calculate the mean grip strength for each time point tested.

### Statistics

All the data were shown as mean±standard error of mean (SEM). Two-tailed Student's t-test (paired or unpaired) was performed to determine the statistical significance between two-group comparison, and the Chi-square test was used to compare the difference of percentage between two groups. One-way ANOVA analysis (GraphPad Prism 7.0) followed by Bonferroni post-hoc test was used to for multiple group comparisons. P<0.05 was considered statistically significant.

### Example 2—A Gene Therapy Approach to Directly Convert Striatal Astrocytes into GABAergic Neurons Coupled with Gene Editing of the Htt Gene

### Design of CRISPR/Cas9 Elements and Production of Recombinant AAV

A target sequence is identified that is complementary to the Htt gene. A guide RNA (gRNA) sequence is designed to target the Htt gene. A donor sequence is designed to modify the number of CAG repeats of the Htt gene to less than 36. The Htt specific gRNA, Cas9 nuclease, and donor sequence is packaged into an AAV vector, for example AAV-Cas9-Htt-P2A-mCherry. The Htt specific gRNA, Cas9 nuclease, and donor sequence may also be packaged in two vectors: AAV-Cas9-P2A-mCherry, AAV-Htt-P2A-mCherry. Recombinant AAV particles is produced as described in Example 1.

### Stereotaxic Viral Injection

Recombinant AAV particles (AAV-Cas9-Htt-P2A-mCherry) is injected into the striatum of R6/2 mice simultaneously with recombinant AAV2/5 from Example 1 (GFAP::Cre, CAG::FLEx-NeuroD1-P2AmCherry, CAG::FLEx-Dlx2-P2A-mCherry). Subjects receiving this combined treatment are tested by behavioral test, such as cat walk, open field test, clasping, mouse weight, and grip strength, as described in Example 1. Behavioral test results are compared against control groups (i) receiving no treatment, (ii) receiving AAV treatment with GFAP::Cre, CAG::FLEx-NeuroD1-P2A-mCherry, CAG::FLEx-Dlx2-P2A-mCherry (from Example 1) alone, and (iii) receiving AAV-Cas9-Htt-P2A-mCherryy to identify synergistic effects.

Recombinant AAV particles (AAV-Cas9-P2A-mCherry and AAV-Htt-P2A-mCherry) is injected into the striatum of R6/2 mice simultaneously with recombinant AAV2/5 from Example 1 (GFAP::Cre, CAG::FLEx-NeuroD1-P2AmCherry, CAG::FLEx-Dlx2-P2A-mCherry). Subjects receiving this combined treatment are tested by behavioral test, such as cat walk, open field test, clasping, mouse weight, and grip strength, as described in Example 1. Behavioral test results are compared against control groups (i) receiving no treatment, (ii) receiving AAV treatment with GFAP::Cre, CAG::FLEx-NeuroD1-P2A-mCherry, CAG::FLEx-Dlx2-P2A-mCherry (from Example 1) alone, and (iii) receiving AAV-Cas9-P2A-mCherry and AAV-Htt-P2A-mCherry to identify synergistic effects.

### Example 3—Additional Embodiments

Embodiment 1. A method for treating a mammal having Huntington's disease, wherein said method comprises:
(a) administering, to glial cells within a striatum of said mammal, nucleic acid encoding a NeuroD1 polypeptide and nucleic acid encoding a Dlx2 polypeptide, wherein said NeuroD1 polypeptide and said Dlx2 polypeptide are expressed by said glial cells, and wherein said glial cells form GABAergic neurons within said striatum; and
(b) administering, to glial cells, neurons, or both within a brain of said mammal, gene therapy components comprising (i) a nuclease or nucleic acid encoding said nuclease, (ii) a targeting nucleic acid sequence complementary to at least a portion of one or both Htt genes, and (iii) a donor nucleic acid comprising at least a fragment of a donor Htt gene comprising a CAG repeat region, wherein said CAG repeat region comprises less than 36 CAG repeats, wherein said donor nucleic acid replaces a sequence of one or both Htt genes present in glial cells, neurons, or both.

Embodiment 2. The method of embodiment 1, wherein said mammal is a human.

Embodiment 3. The method of any one of embodiments 1-2, wherein said glial cells of step (a) are astrocytes.

Embodiment 4. The method of any one of embodiments 1-3, wherein said GABAergic neurons are DARPP32-positive.

Embodiment 5. The method of any one of embodiments 1-4, wherein said GABAergic neurons comprise axonal projections that extend out of said striatum.

Embodiment 6. The method of embodiment 5, wherein said axonal projections extend into the globus pallidus (GP) of said mammal.

Embodiment 7. The method of embodiment 5, wherein said axonal projections extend into the substantia nigra pars reticulata (SNr) of said mammal.

Embodiment 8. The method of any one of embodiments 1-7, wherein said NeuroD1 polypeptide is a human NeuroD1 polypeptide or wherein said Dlx2 polypeptide is a human Dlx2 polypeptide.

Embodiment 9. The method of any one of embodiments 1-8, wherein said nucleic acid encoding said NeuroD1 polypeptide or said nucleic acid encoding said Dlx2 polypeptide is administered to said glial cells in the form of a viral vector.

Embodiment 10. The method of embodiment 9, wherein said viral vector is an adeno-associated viral vector.

Embodiment 11. The method of embodiment 10, wherein said adeno-associated viral vector is an adeno-associated serotype 2/5 viral vector.

Embodiment 12. The method of any one of embodiments 1-11, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 13. The method of any one of embodiments 1-11, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 14. The method of any one of embodiments 1-13, wherein said nucleic acid encoding said NeuroD1 polypeptide or said nucleic acid encoding said Dlx2 polypeptide is operably linked to a promoter sequence.

Embodiment 15. The method of any one of embodiments 1-14, wherein said nuclease is a CRISPR-associated (Cas) nuclease, and wherein said targeting nucleic acid sequence is a guide RNA (gRNA).

Embodiment 16. The method of any one of embodiment 1-14, wherein said nuclease is selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and wherein said targeting nucleic acid sequence is a transcription activator-like (TAL) effector DNA-binding domain.

Embodiment 17. The method of any one of embodiments 1-16, wherein said administration of said nucleic acid encoding a NeuroD1 polypeptide and said nucleic acid encoding a Dlx2 polypeptide or said administration of said gene therapy components comprise a direct injection into said striatum.

Embodiment 18. The method of any one of embodiments 1-16, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 19. The method of any one of embodiments 1-18, wherein said method comprises, prior to said administering steps, identifying said mammal as having Huntington's disease.

Embodiment 20. A method for treating a mammal having Huntington's disease, wherein said mammal is heterozygous for an Htt allele having more than 36 CAG repeats, wherein said method comprises:
(a) administering, to glial cells within a striatum of said mammal, nucleic acid encoding a NeuroD1 polypeptide and nucleic acid encoding a Dlx2 polypeptide, wherein said NeuroD1 polypeptide and said Dlx2 polypeptide are expressed by said glial cells, and wherein said glial cells form GABAergic neurons within said striatum; and
(b) administering, to glial cells, neurons, or both within a brain of said mammal, a composition comprising (i) a nuclease or nucleic acid encoding said nuclease and (ii) a targeting nucleic acid sequence complementary to at least a portion of said Htt allele, wherein said composition edits said Htt allele of glial cells, neurons, or both to form an edited Htt allele, and wherein said edited Htt allele is unable to express a polypeptide comprising more than 11 consecutive glutamine residues.

Embodiment 21. The method of embodiment 20, wherein said mammal is a human.

Embodiment 22. The method of any one of embodiments 20-21, wherein said glial cells of step (a) are astrocytes.

Embodiment 23. The method of any one of embodiments 20-22, wherein said GABAergic neurons are DARPP32-positive.

Embodiment 24. The method of any one of embodiments 20-23, wherein said GABAergic neurons comprise axonal projections that extend out of said striatum.

Embodiment 25. The method of embodiment 24, wherein said axonal projections extend into the GP of said mammal.

Embodiment 26. The method of embodiment 24, wherein said axonal projections extend into the SNr of said mammal.

Embodiment 27. The method of any one of embodiments 20-26, wherein said NeuroD1 polypeptide is a human NeuroD1 polypeptide or wherein said Dlx2 polypeptide is a human Dlx2 polypeptide.

Embodiment 28. The method of any one of embodiments 20-27, wherein said nucleic acid encoding said NeuroD1 polypeptide or said nucleic acid encoding said Dlx2 polypeptide is administered to said glial cells in the form of a viral vector.

Embodiment 29. The method of embodiment 28, wherein said viral vector is an adeno-associated viral vector.

Embodiment 30. The method of embodiment 29, wherein said adeno-associated viral vector is an adeno-associated serotype 2/5 viral vector.

Embodiment 31. The method of any one of embodiments 20-30, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 32. The method of any one of embodiments 20-30, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 33. The method of any one of embodiments 20-32, wherein said nucleic acid encoding said NeuroD1 polypeptide or said nucleic acid encoding said Dlx2 polypeptide is operably linked to a promoter sequence.

Embodiment 34. The method of any one of embodiments 20-33, wherein said nuclease is a Cas nuclease, and wherein said targeting nucleic acid sequence is a gRNA.

Embodiment 35. The method of any one of embodiments 20-33, wherein said nuclease is selected from the group consisting of a FokI nuclease, a HhaI nuclease, a HindIII nuclease, a NotI nuclease, a BbvCI nuclease, an EcoRI nuclease, a BO nuclease, and an AlwI nuclease; and wherein said targeting nucleic acid sequence is a TAL effector DNA-binding domain.

Embodiment 36. The method of any one of embodiments 20-35, wherein said administration of said nucleic acid encoding a NeuroD1 polypeptide and said nucleic acid encoding a Dlx2 polypeptide or said administration of said gene therapy components comprise a direct injection into said brain.

Embodiment 37. The method of any one of embodiments 20-35, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 38. The method of any one of embodiments 20-37, wherein said method comprises, prior to said administering steps, identifying said mammal as having Huntington's disease.

Embodiment 39. A method for improving a motor function in a mammal having Huntington's disease, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering gene therapy components to glial cells, neurons, or both within a brain of said mammal, wherein said gene therapy components reduce the number of CAG repeats in one or both Htt genes present in glial cells, neurons, or both to less than 36 CAG repeats.

Embodiment 40. The method of embodiment 39, wherein said motor function is selected from the group consisting of fine motor skills, tremors, seizures, chorea, dystonia, dyskinesia, slow or abnormal eye movements, impaired gait, impaired posture, impaired balance, difficulty with speech, difficulty with swallowing, difficulty organizing, difficulty prioritizing, difficulty focusing on tasks, lack of flexibility, lack of impulse control, outbursts, lack of awareness of one's own behaviors and/or abilities, slowness in processing thoughts, difficulty in learning new information, depression, irritability, sadness or apathy, social withdrawal, insomnia, fatigue, lack of energy, obsessive-compulsive disorder, mania, bipolar disorder, and weight loss.

Embodiment 41. The method of any one of embodiments 39-40, wherein said mammal is a human.

Embodiment 42. The method of any one of embodiments 39-41, wherein said glial cells of step (a) are astrocytes.

Embodiment 43. The method of any one of embodiments 39-42, wherein said GABAergic neurons are DARPP32-positive.

Embodiment 44. The method of any one of embodiments 39-43, wherein said GABAergic neurons comprise axonal projections that extend out of said striatum.

Embodiment 45. The method of embodiment 44, wherein said axonal projections extend into the GP of said mammal.

Embodiment 46. The method of embodiment 44, wherein said axonal projections extend into the SNr of said mammal.

Embodiment 47. The method of any one of embodiments 39-46, wherein said NeuroD1 polypeptide is a human NeuroD1 polypeptide or wherein said Dlx2 polypeptide is a human Dlx2 polypeptide.

Embodiment 48. The method of any one of embodiments 39-47, wherein said nucleic acid encoding said NeuroD1 polypeptide or said nucleic acid encoding said Dlx2 polypeptide is administered to said glial cells in the form of a viral vector.

Embodiment 49. The method of embodiment 48, wherein said viral vector is an adeno-associated viral vector.

Embodiment 50. The method of embodiment 50, wherein said adeno-associated viral vector is an adeno-associated serotype 2/5 viral vector.

Embodiment 51. The method of any one of embodiments 39-50, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 52. The method of any one of embodiments 39-50, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 53. The method of any one of embodiments 39-52, wherein said nucleic acid encoding said NeuroD1 polypeptide or said nucleic acid encoding said Dlx2 polypeptide is operably linked to a promoter sequence.

Embodiment 54. The method of any one of embodiments 39-53, wherein said gene therapy components comprise (i) a nuclease or nucleic acid encoding said nuclease, (ii) a targeting nucleic acid sequence complementary to at least a portion of one or both Htt genes, and (iii) a donor nucleic acid comprising at least a fragment of a donor Htt gene comprising less than 36 CAG repeats.

Embodiment 55. The method of embodiment 54, wherein said nuclease is a Cas nuclease, and wherein said targeting nucleic acid sequence is a gRNA.

Embodiment 56. The method of embodiment 54, wherein said nuclease is selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and wherein said targeting nucleic acid sequence is a TAL effector DNA-binding domain.

Embodiment 57. The method of any one of embodiments 39-56, wherein said administration of said nucleic acid encoding a NeuroD1 polypeptide and said nucleic acid encoding a Dlx2 polypeptide or said administration of said gene therapy components comprise a direct injection into said brain.

Embodiment 58. The method of any one of embodiments 39-56, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 59. The method of any one of embodiments 39-58, wherein said method comprises, prior to said administering steps, identifying said mammal as having Huntington's disease.

Embodiment 60. A method for improving a motor function in a mammal having Huntington's disease, wherein said mammal is heterozygous for an Htt allele having more than 36 CAG repeats, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering, to glial cells, neurons, or both within a brain of said mammal, a composition comprising (i) a nuclease or nucleic acid encoding said nuclease and (ii) a targeting nucleic acid sequence complementary to at least a portion of said Htt allele, wherein said composition edits said Htt allele of glial cells, neurons, or both to form an edited Htt allele, and wherein said edited Htt allele is unable to express a polypeptide comprising more than 11 consecutive glutamine residues.

Embodiment 61. The method of embodiment 60, wherein said motor function is selected from the group consisting of fine motor skills, tremors, seizures, chorea, dystonia, dyskinesia, slow or abnormal eye movements, impaired gait, impaired posture, impaired balance, difficulty with speech, difficulty with swallowing, difficulty organizing, difficulty prioritizing, difficulty focusing on tasks, lack of flexibility, lack of impulse control, outbursts, lack of awareness of one's own behaviors and/or abilities, slowness in processing thoughts, difficulty in learning new information, depression, irritability, sadness or apathy, social withdrawal, insomnia, fatigue, lack of energy, obsessive-compulsive disorder, mania, bipolar disorder, and weight loss.

Embodiment 62. The method of any one of embodiments 60-61, wherein said mammal is a human.

Embodiment 63. The method of any one of embodiments 60-62, wherein said glial cells of step (a) are astrocytes.

Embodiment 64. The method of any one of embodiments 60-63, wherein said GABAergic neurons are DARPP32-positive.

Embodiment 65. The method of any one of embodiments 60-64, wherein said GABAergic neurons comprise axonal projections that extend out of said striatum.

Embodiment 66. The method of embodiment 65, wherein said axonal projections extend into the GP of said mammal.

Embodiment 67. The method of embodiment 65, wherein said axonal projections extend into the SNr of said mammal.

Embodiment 68. The method of any one of embodiments 60-67, wherein said NeuroD1 polypeptide is a human NeuroD1 polypeptide or wherein said Dlx2 polypeptide is a human Dlx2 polypeptide.

Embodiment 69. The method of any one of embodiments 60-68, wherein said nucleic acid encoding said NeuroD1 polypeptide or said nucleic acid encoding said Dlx2 polypeptide is administered to said glial cells in the form of a viral vector.

Embodiment 70. The method of embodiment 69, wherein said viral vector is an adeno-associated viral vector.

Embodiment 71. The method of embodiment 70, wherein said adeno-associated viral vector is an adeno-associated serotype 2/5 viral vector.

Embodiment 72. The method of any one of embodiments 60-71, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 73. The method of any one of embodiments 60-71, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 74. The method of any one of embodiments 60-73, wherein said nucleic acid encoding said NeuroD1 polypeptide or said nucleic acid encoding said Dlx2 polypeptide is operably linked to a promoter sequence.

Embodiment 75. The method of any one of embodiments 60-74, wherein said nuclease is a Cas nuclease, and wherein said targeting nucleic acid sequence is a gRNA.

Embodiment 76. The method of any one of embodiments 60-74, wherein said nuclease is selected from the group consisting of a FokI nuclease, a HhaI nuclease, a HindIII nuclease, a NotI nuclease, a BbvCI nuclease, an EcoRI nuclease, a BO nuclease, and an AlwI nuclease; and wherein said targeting nucleic acid sequence is a TAL effector DNA-binding domain.

Embodiment 77. The method of any one of embodiments 60-76, wherein said administration of said nucleic acid encoding a NeuroD1 polypeptide and said nucleic acid encoding a Dlx2 polypeptide or said administration of said gene therapy components comprise a direct injection into said brain.

Embodiment 78. The method of any one of embodiments 60-77, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 79. The method of any one of embodiments 60-78, wherein said method comprises, prior to said administering steps, identifying said mammal as having Huntington's disease.

Embodiment 80. A method for improving life expectancy of a mammal having Huntington's disease, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering gene therapy components to glial cells, neurons, or both within a brain of said mammal, wherein said gene therapy components reduce the number of CAG repeats in one or both Htt genes present in glial cells, neurons, or both to less than 36 CAG repeats.

Embodiment 81. The method of embodiment 80, wherein said life expectancy of said mammal is extended by from about 10% to about 60%.

Embodiment 82. The method of any one of embodiments 80-81, wherein said mammal is a human.

Embodiment 83. The method of any one of embodiments 80-82, wherein said glial cells of step (a) are astrocytes.

Embodiment 84. The method of any one of embodiments 80-83, wherein said GABAergic neurons are DARPP32-positive.

Embodiment 85. The method of any one of embodiments 80-84, wherein said GABAergic neurons comprise axonal projections that extend out of said striatum.

Embodiment 86. The method of embodiment 85, wherein said axonal projections extend into the GP of said mammal.

Embodiment 87. The method of embodiment 85, wherein said axonal projections extend into the SNr of said mammal.

Embodiment 88. The method of any one of embodiments 80-87, wherein said NeuroD1 polypeptide is a human NeuroD1 polypeptide or wherein said Dlx2 polypeptide is a human Dlx2 polypeptide.

Embodiment 89. The method of any one of embodiments 80-88, wherein said nucleic acid encoding said NeuroD1 polypeptide or said nucleic acid encoding said Dlx2 polypeptide is administered to said glial cells in the form of a viral vector.

Embodiment 90. The method of embodiment 89, wherein said viral vector is an adeno-associated viral vector.

Embodiment 91. The method of embodiment 90, wherein said adeno-associated viral vector is an adeno-associated serotype 2/5 viral vector.

Embodiment 92. The method of any one of embodiments 80-91, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 93. The method of any one of embodiments 80-91, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 94. The method of any one of embodiments 80-93, wherein said nucleic acid encoding said NeuroD1 polypeptide or said nucleic acid encoding said Dlx2 polypeptide is operably linked to a promoter sequence.

Embodiment 95. The method of any one of embodiments 80-94, wherein said gene therapy components comprise (i) a nuclease or nucleic acid encoding said nuclease, (ii) a targeting nucleic acid sequence complementary to at least a portion of one or both Htt genes, and (iii) a donor nucleic acid comprising at least a fragment of a donor Htt gene comprising less than 36 CAG repeats.

Embodiment 96. The method of embodiment 95, wherein said nuclease is a Cas nuclease, and wherein said targeting nucleic acid sequence is a gRNA.

Embodiment 97. The method of embodiment 95, wherein said nuclease is selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and wherein said targeting nucleic acid sequence is a TAL effector DNA-binding domain.

Embodiment 98. The method of any one of embodiments 80-97, wherein said administration of said nucleic acid encoding a NeuroD1 polypeptide and said nucleic acid encoding a Dlx2 polypeptide or said administration of said gene therapy components comprise a direct injection into said brain.

Embodiment 99. The method of any one of embodiments 80-97, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 100. The method of any one of embodiments 80-99, wherein said method comprises, prior to said administering steps, identifying said mammal as having Huntington's disease.

Embodiment 101. A method for improving life expectancy of a mammal having Huntington's disease, wherein said mammal is heterozygous for an Htt allele having more than 36 CAG repeats, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering, to glial cells, neurons, or both within a brain of said mammal, a composition comprising (i) a nuclease or nucleic acid encoding said nuclease and (ii) a targeting nucleic acid sequence complementary to at least a portion of said Htt allele, wherein said composition edits said Htt allele of glial cells, neurons, or both to form an edited Htt allele, and wherein said edited Htt allele is unable to express a polypeptide comprising more than 11 consecutive glutamine residues.

Embodiment 102. The method of embodiment 101, wherein said life expectancy of said mammal is extended by from about 10% to about 60%.

Embodiment 103. The method of any one of embodiments 101-102, wherein said mammal is a human.

Embodiment 104. The method of any one of embodiments 101-103, wherein said glial cells of step (a) are astrocytes.

Embodiment 105. The method of any one of embodiments 101-104, wherein said GABAergic neurons are DARPP32-positive.

Embodiment 106. The method of any one of embodiments 101-105, wherein said GABAergic neurons comprise axonal projections that extend out of said striatum.

Embodiment 107. The method of embodiment 106, wherein said axonal projections extend into the GP of said mammal.

Embodiment 108. The method of embodiment 106, wherein said axonal projections extend into the SNr of said mammal.

Embodiment 109. The method of any one of embodiments 101-108, wherein said NeuroD1 polypeptide is a human NeuroD1 polypeptide or wherein said Dlx2 polypeptide is a human Dlx2 polypeptide.

Embodiment 110. The method of any one of embodiments 101-109, wherein said nucleic acid encoding said NeuroD1 polypeptide or said nucleic acid encoding said Dlx2 polypeptide is administered to said glial cells in the form of a viral vector.

Embodiment 111. The method of embodiment 110, wherein said viral vector is an adeno-associated viral vector.

Embodiment 112. The method of embodiment 111, wherein said adeno-associated viral vector is an adeno-associated serotype 2/5 viral vector.

Embodiment 113. The method of any one of embodiments 101-112, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 114. The method of any one of embodiments 101-112, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 115. The method of any one of embodiments 101-114, wherein said nucleic acid encoding said NeuroD1 polypeptide or said nucleic acid encoding said Dlx2 polypeptide is operably linked to a promoter sequence.

Embodiment 116. The method of embodiment 115, wherein said nuclease is a Cas nuclease, and wherein said targeting nucleic acid sequence is a gRNA.

Embodiment 117. The method of embodiment 115, wherein said nuclease is selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and wherein said targeting nucleic acid sequence is a TAL effector DNA-binding domain.

Embodiment 118. The method of any one of embodiments 101-117, wherein said administration of said nucleic acid encoding a NeuroD1 polypeptide and said nucleic acid encoding a Dlx2 polypeptide or said administration of said gene therapy components comprise a direct injection into said brain.

Embodiment 119. The method of any one of embodiments 101-118, wherein said administration of said nucleic acid encoding a NeuroD1 polypeptide and said nucleic acid encoding a Dlx2 polypeptide or said administration of said gene therapy components comprise an intraperitoneal, intramuscular, intrathecal, intracerebral, intraparenchymal, intravenous, intranasal, or oral administration.

Embodiment 120. The method of any one of embodiments 101-119, wherein said method comprises, prior to said administering steps, identifying said mammal as having Huntington's disease.

Embodiment 121. A method for reducing striatum atrophy in a mammal having Huntington's disease, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering gene therapy components to glial cells, neurons, or both within a brain of said mammal, wherein said gene therapy components reduce the number of CAG repeats in one or both Htt genes present in glial cells, neurons, or both to less than 36 CAG repeats.

Embodiment 122. The method of embodiment 121, wherein said mammal is a human.

Embodiment 123. The method of any one of embodiments 121-122, wherein said glial cells of step (a) are astrocytes.

Embodiment 124. The method of any one of embodiments 121-123, wherein said GABAergic neurons are DARPP32-positive.

Embodiment 125. The method of any one of embodiments 121-124, wherein said GABAergic neurons comprise axonal projections that extend out of said striatum.

Embodiment 126. The method of embodiment 125, wherein said axonal projections extend into the GP of said mammal.

Embodiment 127. The method of embodiment 125, wherein said axonal projections extend into the SNr of said mammal.

Embodiment 128. The method of any one of embodiments 121-127, wherein said NeuroD1 polypeptide is a human NeuroD1 polypeptide or wherein said Dlx2 polypeptide is a human Dlx2 polypeptide.

Embodiment 129. The method of any one of embodiments 121-128, wherein said nucleic acid encoding said NeuroD1 polypeptide or said nucleic acid encoding said Dlx2 polypeptide is administered to said glial cells in the form of a viral vector.

Embodiment 130. The method of embodiment 129, wherein said viral vector is an adeno-associated viral vector.

Embodiment 131. The method of embodiment 130, wherein said adeno-associated viral vector is an adeno-associated serotype 2/5 viral vector.

Embodiment 132. The method of any one of embodiments 121-131, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 133. The method of any one of embodiments 121-131, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 134. The method of any one of embodiments 121-133, wherein said nucleic acid encoding said NeuroD1 polypeptide or said nucleic acid encoding said Dlx2 polypeptide is operably linked to a promoter sequence.

Embodiment 135. The method of any one of embodiments 121-134, wherein said gene therapy components comprise (i) a nuclease or nucleic acid encoding said nuclease, and (ii) a targeting nucleic acid sequence complementary to at least a portion of one or both Htt genes.

Embodiment 136. The method of embodiment 135, wherein said nuclease is a Cas nuclease, and wherein said targeting nucleic acid sequence is a gRNA.

Embodiment 137. The method of embodiment 135, wherein said nuclease is selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and wherein said targeting nucleic acid sequence is a TAL effector DNA-binding domain.

Embodiment 138. The method of any one of embodiments 121-137, wherein said administration of said nucleic acid encoding a NeuroD1 polypeptide and said nucleic acid encoding a Dlx2 polypeptide or said administration of said gene therapy components comprise a direct injection into said brain.

Embodiment 139. The method of any one of embodiments 121-137, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 140. The method of any one of embodiments 121-139, wherein said method comprises, prior to said administering steps, identifying said mammal as having Huntington's disease.

Embodiment 141. A method for reducing striatum atrophy in a mammal having Huntington's disease, wherein said mammal is heterozygous for an Htt allele having more than 36 CAG repeats, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering, to glial cells, neurons, or both within a brain of said mammal, a composition comprising (i) a nuclease or nucleic acid encoding said nuclease and (ii) a targeting nucleic acid sequence complementary to at least a portion of said Htt allele, wherein said composition edits said Htt allele of glial cells, neurons, or both to form an edited Htt allele, and wherein said edited Htt allele is unable to express a polypeptide comprising more than 11 consecutive glutamine residues.

Embodiment 142. The method of embodiment 141, wherein said mammal is a human.

Embodiment 143. The method of any one of embodiments 141-142, wherein said glial cells of step (a) are astrocytes.

Embodiment 144. The method of any one of embodiments 141-143, wherein said GABAergic neurons are DARPP32-positive.

Embodiment 145. The method of any one of embodiments 141-144, wherein said GABAergic neurons comprise axonal projections that extend out of said striatum.

Embodiment 146. The method of embodiment 145, wherein said axonal projections extend into the GP of said mammal.

Embodiment 147. The method of embodiment 145, wherein said axonal projections extend into the SNr of said mammal.

Embodiment 148. The method of any one of embodiments 141-147, wherein said NeuroD1 polypeptide is a human NeuroD1 polypeptide or wherein said Dlx2 polypeptide is a human Dlx2 polypeptide.

Embodiment 149. The method of any one of embodiments 141-148, wherein said nucleic acid encoding said NeuroD1 polypeptide or said nucleic acid encoding said Dlx2 polypeptide is administered to said glial cells in the form of a viral vector.

Embodiment 150. The method of embodiment 149, wherein said viral vector is an adeno-associated viral vector.

Embodiment 151. The method of embodiment 150, wherein said adeno-associated viral vector is an adeno-associated serotype 2/5 viral vector.

Embodiment 152. The method of any one of embodiments 141-151, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 153. The method of any one of embodiments 141-151, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 154. The method of any one of embodiments 141-453, wherein said nucleic acid encoding said NeuroD1 polypeptide or said nucleic acid encoding said Dlx2 polypeptide is operably linked to a promoter sequence.

Embodiment 155. The method of any one of embodiments 141-153, wherein said nuclease is a Cas nuclease, and wherein said targeting nucleic acid sequence is a gRNA.

Embodiment 156. The method of any one of embodiments 141-153, wherein said nuclease is selected from the group consisting of a FokI nuclease, a HhaI nuclease, a HindIII nuclease, a NotI nuclease, a BbvCI nuclease, an EcoRI nuclease, a BO nuclease, and an AlwI nuclease; and wherein said targeting nucleic acid sequence is a TAL effector DNA-binding domain.

Embodiment 157. The method of any one of embodiments 141-156, wherein said administration of said nucleic acid encoding a NeuroD1 polypeptide and said nucleic acid encoding a Dlx2 polypeptide or said administration of said gene therapy components comprise a direct injection into said brain.

Embodiment 158. The method of any one of embodiments 141-157, wherein said administration of said nucleic acid encoding a NeuroD1 polypeptide and said nucleic acid encoding a Dlx2 polypeptide or said administration of said gene therapy components comprise an intraperitoneal, intramuscular, intrathecal, intracerebral, intraparenchymal, intravenous, intranasal, or oral administration.

Embodiment 159. The method of any one of embodiments 140-157, wherein said method comprises, prior to said administering steps, identifying said mammal as having Huntington's disease.

Embodiment 160. A method for reducing nuclear HTT polypeptide inclusions in a mammal having Huntington's disease, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering gene therapy components to glial cells, neurons, or both within a brain of said mammal, wherein said gene therapy components reduce the number of CAG repeats in one or both Htt genes present in glial cells, neurons, or both to less than 36 CAG repeats.

Embodiment 161. The method of embodiment 160, wherein said mammal is a human.

Embodiment 162. The method of any one of embodiments 160-161, wherein said glial cells of step (a) are astrocytes.

Embodiment 163. The method of any one of embodiments 160-162, wherein said GABAergic neurons are DARPP32-positive.

Embodiment 164. The method of any one of embodiments 160-163, wherein said GABAergic neurons comprise axonal projections that extend out of said striatum.

Embodiment 165 The method of embodiment 164, wherein said axonal projections extend into the GP of said mammal.

Embodiment 166. The method of embodiment 164, wherein said axonal projections extend into the SNr of said mammal.

Embodiment 167. The method of any one of embodiments 160-166, wherein said NeuroD1 polypeptide is a human NeuroD1 polypeptide or wherein said Dlx2 polypeptide is a human Dlx2 polypeptide.

Embodiment 168. The method of any one of embodiments 160-167, wherein said nucleic acid encoding said NeuroD1 polypeptide or said nucleic acid encoding said Dlx2 polypeptide is administered to said glial cells in the form of a viral vector.

Embodiment 169. The method of embodiment 168, wherein said viral vector is an adeno-associated viral vector.

Embodiment 170. The method of embodiment 169, wherein said adeno-associated viral vector is an adeno-associated serotype 2/5 viral vector.

Embodiment 171. The method of any one of embodiments 160-170, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 172. The method of any one of embodiments 160-171, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 173. The method of any one of embodiments 160-172, wherein said nucleic acid encoding said NeuroD1 polypeptide or said nucleic acid encoding said Dlx2 polypeptide is operably linked to a promoter sequence.

Embodiment 174. The method of any one of embodiments 160-173, wherein said gene therapy components comprise (i) a nuclease or nucleic acid encoding said nuclease, (ii) a targeting nucleic acid sequence complementary to at least a portion of one or both Htt genes, and (iii) a donor nucleic acid comprising at least a fragment of a donor Htt gene comprising less than 36 CAG repeats.

Embodiment 175. The method of embodiment 174, wherein said nuclease is a Cas nuclease, and wherein said targeting nucleic acid sequence is a gRNA.

Embodiment 176. The method of embodiment 174, wherein said nuclease is selected from the group consisting of a FokI nuclease, a HhaI nuclease, a HindIII nuclease, a Nod nuclease, a BbvCI nuclease, an EcoRI nuclease, a BglI nuclease, and an AlwI nuclease; and wherein said targeting nucleic acid sequence is a TAL effector DNA-binding domain.

Embodiment 177. The method of any one of embodiments 160-176, wherein said administration of said nucleic acid encoding a NeuroD1 polypeptide and said nucleic acid encoding a Dlx2 polypeptide or said administration of said gene therapy components comprise a direct injection into said brain.

Embodiment 178. The method of any one of embodiments 160-177, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 179. The method of any one of embodiments 160-178, wherein said method comprises, prior to said administering steps, identifying said mammal as having Huntington's disease.

Embodiment 180. A method for reducing nuclear HTT polypeptide inclusions in a mammal having Huntington's disease, wherein said mammal is heterozygous for an Htt allele having more than 36 CAG repeats, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering, to glial cells, neurons, or both within a brain of said mammal, a composition comprising (i) a nuclease or nucleic acid encoding said nuclease and (ii) a targeting nucleic acid sequence complementary to at least a portion of said Htt allele, wherein said composition edits said Htt allele of glial cells, neurons, or both to form an edited Htt allele, and wherein said edited Htt allele is unable to express a polypeptide comprising more than 11 consecutive glutamine residues.

Embodiment 181. The method of embodiment 180, wherein said mammal is a human.

Embodiment 182. The method of any one of embodiments 180-181, wherein said glial cells of step (a) are astrocytes.

Embodiment 183. The method of any one of embodiments 180-182, wherein said GABAergic neurons are DARPP32-positive.

Embodiment 184. The method of any one of embodiments 180-183, wherein said GABAergic neurons comprise axonal projections that extend out of said striatum.

Embodiment 185. The method of embodiment 184, wherein said axonal projections extend into the GP of said mammal.

Embodiment 186. The method of embodiment 184, wherein said axonal projections extend into the SNr of said mammal.

Embodiment 187. The method of any one of embodiments 180-186, wherein said NeuroD1 polypeptide is a human NeuroD1 polypeptide or wherein said Dlx2 polypeptide is a human Dlx2 polypeptide.

Embodiment 188. The method of any one of embodiments 180-187, wherein said nucleic acid encoding said NeuroD1 polypeptide or said nucleic acid encoding said Dlx2 polypeptide is administered to said glial cells in the form of a viral vector.

Embodiment 189. The method of embodiment 188, wherein said viral vector is an adeno-associated viral vector.

Embodiment 190. The method of embodiment 189, wherein said adeno-associated viral vector is an adeno-associated serotype 2/5 viral vector.

Embodiment 191. The method of any one of embodiments 180-190, wherein said nucleic acid encoding said NeuroD1 polypeptide and said nucleic acid encoding said Dlx2 polypeptide are located on the same viral vector, and wherein said viral vector is administered to said glial cells of step (a).

Embodiment 192. The method of any one of embodiments 180-190, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

Embodiment 193. The method of any one of embodiments 180-192, wherein said nucleic acid encoding said NeuroD1 polypeptide or said nucleic acid encoding said Dlx2 polypeptide is operably linked to a promoter sequence.

Embodiment 194. The method of any one of embodiments 180-196, wherein said nuclease is a Cas nuclease, and wherein said targeting nucleic acid sequence is a gRNA.

Embodiment 195. The method of any one of embodiments 180-196, wherein said nuclease is selected from the group consisting of a FokI nuclease, a HhaI nuclease, a HindIII nuclease, a NotI nuclease, a BbvCI nuclease, an EcoRI nuclease, a BO nuclease, and an AlwI nuclease; and wherein said targeting nucleic acid sequence is a TAL effector DNA-binding domain.

Embodiment 196. The method of any one of embodiments 180-195, wherein said administration of said nucleic acid encoding a NeuroD1 polypeptide and said nucleic acid encoding a Dlx2 polypeptide or said administration of said gene therapy components comprise a direct injection into said brain.

Embodiment 197. The method of any one of embodiments 180-195, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

Embodiment 198. The method of any one of embodiments 180-197, wherein said method comprises, prior to said administering steps, identifying said mammal as having Huntington's disease.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for improving a motor function in a mammal having Huntington's disease, wherein said method comprises:
(a) administering nucleic acid encoding a NeuroD1 polypeptide and nucleic acid encoding a Dlx2 polypeptide to glial cells within a striatum of said mammal, wherein said NeuroD1 polypeptide and said Dlx2 polypeptide are expressed by said glial cells, and wherein said glial cells form GABAergic neurons within said striatum; and
(b) administering gene therapy components to glial cells, neurons, or both within a brain of said mammal, wherein said gene therapy components reduce the number of CAG repeats in one or both Htt genes present in glial cells, neurons, or both to less than 36 CAG repeats.

2. The method of claim 1, wherein said motor function is selected from the group consisting of tremors and seizures.

3. The method of claim 1, wherein said mammal is a human.

4. The method of claim 1, wherein said glial cells of step (a) are astrocytes.

5. The method of claim 1, wherein said GABAergic neurons are DARPP32-positive.

6. The method of claim 1, wherein said GABAergic neurons comprise axonal projections that extend out of said striatum.

7. The method of claim 6, wherein said axonal projections extend into the GP of said mammal.

8. The method of claim 6, wherein said axonal projections extend into the SNr of said mammal.

9. The method of claim 1, wherein said NeuroD1 polypeptide is a human NeuroD1 polypeptide or wherein said Dlx2 polypeptide is a human Dlx2 polypeptide.

10. The method of claim 1, wherein said nucleic acid encoding said NeuroD1 polypeptide or said nucleic acid encoding said Dlx2 polypeptide is administered to said glial cells in the form of a viral vector.

11. The method of claim 10, wherein said viral vector is an adeno-associated viral vector.

12. The method of claim 11, wherein said adeno-associated viral vector is an adeno-associated serotype 2/5 viral vector.

13. The method of claim 1, wherein said nucleic acid encoding said NeuroD1 polypeptide and said nucleic acid encoding said Dlx2 polypeptide are located on the same viral vector, and wherein said viral vector is administered to said glial cells of step (a).

14. The method of claim 1, wherein said nucleic acid encoding said NeuroD1 polypeptide and said nucleic acid encoding said Dlx2 polypeptide are located on separate viral vectors, and wherein each of said separate viral vectors is administered to said glial cells of step (a).

15. The method of claim 1, wherein said nucleic acid encoding said NeuroD1 polypeptide or said nucleic acid encoding said Dlx2 polypeptide is operably linked to a promoter sequence.

16. The method of claim 1, wherein said gene therapy components comprise (i) a nuclease or nucleic acid encoding said nuclease, (ii) a targeting nucleic acid sequence complementary to at least a portion of one or both Htt genes, and (iii) a donor nucleic acid comprising at least a fragment of a donor Htt gene comprising less than 36 CAG repeats.

17. The method of claim 16, wherein said nuclease is a Cas nuclease, and wherein said targeting nucleic acid sequence is a gRNA.

18. The method of claim 16, wherein said nuclease is selected from the group consisting of a FokI nuclease, a HhaI nuclease, a HindIII nuclease, a NotI nuclease, a BbvCI nuclease, an EcoRI nuclease, a BglI nuclease, and an AlwI nuclease; and wherein said targeting nucleic acid sequence is a TAL effector DNA-binding domain.

19. The method of claim 1, wherein said administration of said nucleic acid encoding a NeuroD1 polypeptide and said nucleic acid encoding a Dlx2 polypeptide or said administration of said gene therapy components comprise a direct injection into said brain.

20. The method of claim 1, wherein said administration of said nucleic acid encoding a NeuroD1 polypeptide and said nucleic acid encoding a Dlx2 polypeptide or said administration of said gene therapy components comprise an intraperitoneal, intramuscular, intravenous, intrathecal, intracerebral, intraparenchymal, intranasal, or oral administration.

21. The method of claim 1, wherein said method comprises, prior to said administering steps, identifying said mammal as having Huntington's disease.
